(19) United States
(12) Patent Application Publication
(10) Pub. No.: US 2010/0173013 A1
(43) Pub. Date: Jul. 8, 2010

(54) TREATMENT OF NEOPLASTIC DISORDERS USING COMBINATION THERAPIES

(76) Inventors: Denis Drygin, San Diego, CA (US); Kenna Anderes, San Diego, CA (US); Caroline B. Ho, Valley Center, CA (US); Joshua R. Bliesath, Escondido, CA (US); Christopher B. Proffitt, Poway, CA (US); Sean O'Brien, Carlsbad, CA (US); William G. Rice, Del Mar, CA (US)

Correspondence Address: COOLEY LLP;ATTN: Patent Group, Suite 1100, 777 - 6th Street, NW, WASHINGTON, DC, 20001, US

(21) Appl. No.: 12/684,053

(22) Filed: Jan. 7, 2010

Related U.S. Application Data
(63) Continuation-in-part of application No. PCT/US2009/046948, filed on Jun. 10, 2009.
(60) Provisional application No. 61/143,282, filed on Jan. 8, 2009.
(60) Provisional application No. 61/228,121, filed on Jul. 23, 2009.
(60) Provisional application No. 61/262,079, filed on Nov. 17, 2009.

(51) Int. Cl. A61K 31/4375; A61K 31/519; A61K 33/24; A61K 31/675; A61K 31/5377; A61P 35/00; A61P 37/06; A61K 31/704; A61K 31/7076; A61K 31/7068; A61K 31/517; A61K 31/437

(52) U.S. Cl. 424/649; 514/267; 514/292; 514/81; 514/233.5; 514/34; 514/48; 514/49; 514/266.24; 514/283

(57) ABSTRACT

The present application is generally directed to compounds, compositions and methods of combination therapy for the treatment of neoplastic disorders.

### RELATED APPLICATIONS

This application claims priority to U.S. Provisional Application No. 61/143,282, filed Jan. 8, 2009, PCT Application No. PCT/US2009/046948, filed Jun. 10, 2009, U.S. Provisional Application No. 61/228,121, filed Jul. 23, 2009, and U.S. Provisional Application No. 61/262,079, filed Nov. 17, 2009, the contents of each of which are hereby incorporated in their entirety by reference.

### FIELD OF TECHNOLOGY

The application is in general directed to methods of combination therapy for neoplastic disorders, and combination pharmaceutical compositions.

Current cancer therapy generally involves treatment with surgery, chemotherapy, radiation therapy, or a combination of these approaches. Each of the major treatment approaches has significant limitations. For example, surgery may not completely remove the neoplastic tissue and cannot be used in the treatment of some disseminated neoplastic conditions, such as acute lymphoblastic leukemia, and radiation therapy is effective only when the irradiated neoplastic tissue exhibits a higher sensitivity to radiation than normal tissue and often causes serious side effects.

While a variety of chemotherapeutic agents are available, nearly all chemotherapeutic agents are toxic, and chemotherapy frequently causes significant, and often dangerous, side effects. Frequent side-effects include severe nausea and vomiting, bone marrow depression, immunosuppression, cytopenia (including, e.g., anemia, neutropenia, and thrombocytopenia), pain and fatigue. Additional side-effects include cachexia, mucositis, alopecia, cutaneous complications (including hypersensitivity reactions, e.g., pruritic, urticaria, and angioedema), as well as neurological, pulmonary, cardiac, reproductive and endocrine complications.

Side effects associated with chemotherapeutic agents are generally the major factor in defining the agent's dose-limiting toxicity (DLT), and managing the adverse side effects induced by chemotherapy and radiation therapy is of major importance in the clinical management of cancer treatment. In addition, many tumor cells are resistant or develop resistance to chemotherapeutic agents through multi-drug resistance.

Combination therapeutic approaches that permit the use of lower doses of chemotherapeutic agents than those conventionally used in monotherapy while maintaining anticancer efficacy are highly desirable. Such combination therapies may lead to a decrease in the frequency and/or severity of adverse side-effects and an improved quality of life for the patient. Further benefits of reducing the incidence of side-effects include improved patient compliance, a reduction in the number of hospitalizations needed for the treatment of adverse effects, and a decrease in the administration of analgesic agents needed to treat pain associated with the adverse effects.

Where dose limiting toxicity is not an issue, combination therapy can also maximize the therapeutic effects of chemotherapeutic agents administered at higher doses. In addition to increased anticancer efficacy, such approaches may reduce the development of resistance.

Compounds of Formula I (as shown herein) have been previously reported to be effective in inhibiting tumor progression. See U.S. Ser. No. 11/849,230 (filed Aug. 31, 2007).

### SUMMARY

The present application provides compounds, compositions and methods of combination therapy using compounds of Formula I for the treatment of neoplastic disorders. It has been found that contacting proliferating cells with commonly used anticancer agents in combination with a compound of Formula I provides a synergistic effect on inhibiting cell proliferation.

In one aspect, the application discloses a method for preventing, treating or ameliorating a neoplastic disorder, comprising administering to a subject in need thereof a therapeutically effective amount of a compound of Formula I:

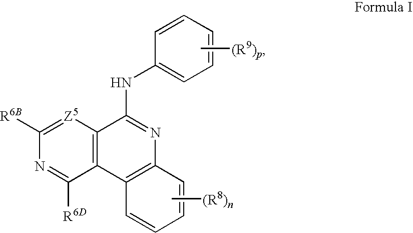

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4;

and an anticancer agent, or a pharmaceutically acceptable salt or ester thereof; thereby preventing, treating or ameliorating said neoplastic disorder.

Anticancer agents used in combination with the compounds of the present application may include agents selected from any of the classes known to those of ordinary skill in the art, including, for example, alkylating agents, anti-metabolites, plant alkaloids and terpenoids (e.g., taxanes), topoisomerase inhibitors, anti-tumor antibiotics, hormonal therapies, molecular targeted agents, and the like. Generally such an anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor.

Another aspect disclosed in the present application is a method for inhibiting cell proliferation in a system comprising administering to the system a compound of Formula I, as disclosed herein, and an anticancer agent or a pharmaceutically acceptable salt or ester thereof, thereby inhibiting cell proliferation.

A further aspect disclosed in the present application is a pharmaceutical composition comprising a compound of Formula I as disclosed herein, an anticancer agent and at least one pharmaceutically acceptable excipient.

### BRIEF DESCRIPTION OF THE FIGURES

FIG. 1 is a graph of the log drug concentration against the relative fluorescent units (RFU) for Compound A and Compound B for calculation of IC₅₀.

FIG. 2 is a graph of the log concentration of Compound K and 5-fluorouracil against RFU for calculation of IC₅₀ for A375 melanoma cells.

FIG. 3 is a bar graph showing the percent cell death for Compound K, 5-fluorouracil and the combination thereof for A375 melanoma cells.

FIG. 4 is a graph of the log concentration of Compound K and fludarabine against RFU for calculation of IC₅₀ for A375 melanoma cells.

FIG. 5 is a bar graph showing the percent cell death for Compound K, fludarabine and the combination thereof for A375 melanoma cells.

FIG. 6 is a graph of the log concentration of Compound K and gemcitabine against RFU for calculation of IC₅₀ for A375 melanoma cells.

FIG. 7 is a graph of the log concentration of Compound K and paclitaxel against RFU for calculation of IC₅₀ for A375 melanoma cells.

FIG. 8 is a bar graph showing the percent cell death for Compound K, paclitaxel and the combination thereof for A375 melanoma cells.

FIG. 9 is a graph of the log concentration of Compound K and sunitinib against RFU for calculation of IC₅₀ for A375 melanoma cells.

FIG. 10 is a bar graph showing the percent cell death for Compound K, sunitinib and the combination thereof for A375 melanoma cells.

FIG. 11 is a graph of the log concentration of Compound K and vinblastine against RFU for calculation of IC₅₀ for A375 melanoma cells.

FIG. 12 is a bar graph showing the percent cell death for Compound K, vinblastine and the combination thereof for A375 melanoma cells.

FIG. 13 is a graph of the log concentration of Compound K and 5-fluorouracil against RFU for calculation of IC₅₀ for MDA-MB-468 breast cancer cells.

FIG. 14 is a bar graph showing the percent cell death for Compound K, 5-fluorouracil and the combination thereof for MDA-MB-468 breast cancer cells.

FIG. 15 is a graph of the log concentration of Compound K and 5-fluorouracil against RFU for calculation of IC₅₀ for MDA-MB-468 breast cancer cells.

FIG. 16 is a bar graph showing the percent cell death for Compound K, 5-fluorouracil and the combination thereof for MDA-MB-468 breast cancer cells.

FIG. 17 is a graph of the log concentration of Compound K and cisplatin against RFU for calculation of IC₅₀ for MDA-MB-468 breast cancer cells.

FIG. 18 is a bar graph showing the percent cell death for Compound K, cisplatin and the combination thereof for MDA-MB-468 breast cancer cells.

FIG. 19 is a graph of the log concentration of Compound K and cisplatin against RFU for calculation of IC₅₀ for MDA-MB-468 breast cancer cells.

FIG. 20 is a bar graph showing the percent cell death for Compound K, cisplatin and the combination thereof for MDA-MB-468 breast cancer cells.

FIG. 21 is a graph of the log concentration of Compound K and doxorubicin against RFU for calculation of IC₅₀ for MDA-MB-468 breast cancer cells.

FIG. 22 is a bar graph showing the percent cell death for Compound K, doxorubicin and the combination thereof for MDA-MB-468 breast cancer cells.

FIG. 23 is a graph of the log concentration of Compound K and doxorubicin against RFU for calculation of IC₅₀ for MDA-MB-468 breast cancer cells.

FIG. 24 is a bar graph showing the percent cell death for Compound K, doxorubicin and the combination thereof for MDA-MB-468 breast cancer cells.

FIG. 25 is a graph of the log concentration of Compound K and gemcitabine against RFU for calculation of IC₅₀ for MDA-MB-468 breast cancer cells.

FIG. 26 is a bar graph showing the percent cell death for Compound K, gemcitabine and the combination thereof for MDA-MB-468 breast cancer cells.

FIG. 27 is a graph of the log concentration of Compound K and gemcitabine against RFU for calculation of IC₅₀ for MDA-MB-468 breast cancer cells.

FIG. 28 is a bar graph showing the percent cell death for Compound K, gemcitabine and the combination thereof for MDA-MB-468 breast cancer cells.

FIG. 29 is a graph of the log concentration of Compound K and vinblastine against RFU for calculation of IC₅₀ for MIA PaCa-2 pancreatic cancer cells.

FIG. 30 is a bar graph showing the percent cell death for Compound K, vinblastine and the combination thereof for MIA PaCa-2 pancreatic cancer cells.

FIG. 31 is a graph of the log concentration of Compound K and gemcitabine against RFU for calculation of IC₅₀ for MIA PaCa-2 pancreatic cancer cells.

FIG. 32 is a bar graph showing the percent cell death for Compound K, gemcitabine and the combination thereof for MIA PaCa-2 pancreatic cancer cells.

FIG. 33 is a graph of the log concentration of Compound K and sunitinib against RFU for calculation of IC₅₀ for MIA PaCa-2 pancreatic cancer cells.

FIG. 34 is a bar graph showing the percent cell death for Compound K, sunitinib and the combination thereof for MIA PaCa-2 pancreatic cancer cells.

FIG. 35 is a graph of the log concentration of Compound K and rapamycin against RFU for calculation of IC₅₀ for MIA PaCa-2 pancreatic cancer cells.

FIG. 36 is a bar graph showing the percent cell death for Compound K, rapamycin and the combination thereof for MIA PaCa-2 pancreatic cancer cells.

FIG. 37 is a graph of the log concentration of Compound K and 5-fluorouracil against RFU for calculation of IC₅₀ for SUM-149PT inflammatory breast carcinoma cells.

FIG. 38 is a bar graph showing the percent cell death for Compound K, 5-fluorouracil and the combination thereof for SUM-149PT inflammatory breast carcinoma cells.

FIG. 39 is a graph of the log concentration of Compound K and cisplatin against RFU for calculation of IC₅₀ for SUM-149PT inflammatory breast carcinoma cells.

FIG. 40 is a bar graph showing the percent cell death for Compound K, cisplatin and the combination thereof for SUM-149PT inflammatory breast carcinoma cells.

FIG. 41 is a graph of the log concentration of Compound K and rapamycin against RFU for calculation of IC₅₀ for SUM-149PT inflammatory breast carcinoma cells.

FIG. 42 is a bar graph showing the percent cell death for Compound K, rapamycin and the combination thereof for SUM-149PT inflammatory breast carcinoma cells.

FIG. 43 is a graph of the log concentration of Compound K and erlotinib against RFU for calculation of IC₅₀ for SUM-149PT inflammatory breast carcinoma cells.

FIG. 44 is a bar graph showing the percent cell death for Compound K, erlotinib and the combination thereof for SUM-149PT inflammatory breast carcinoma cells.

FIG. 45 is a graph of the log concentration of Compound K and 5-fluorouracil against RFU for calculation of IC₅₀ for SUM-190PT inflammatory breast carcinoma cells.

FIG. 46 is a bar graph showing the percent cell death for Compound K, 5-fluorouracil and the combination thereof for SUM-190PT inflammatory breast carcinoma cells.

FIG. 47 is a dose response curve for Compound K, erlotinib and the combination thereof for BT-474 breast carcinoma cells.

FIG. 48 is a bar graph showing the percent cell death for Compound K, erlotinib and the combination thereof for BT-474 breast carcinoma cells.

FIG. 49 is a dose response curve of Compound K and Compound K in combination with erlotinib for erlotinib-resistant MDA-MB-453 breast carcinoma cells.

FIG. 50 is a dose response curve of erlotinib for erlotinib-resistant MDA-MB-453 breast carcinoma cells.

FIG. 51 is a bar graph showing the percent cell death for Compound K, erlotinib and the combination thereof for erlotinib-resistant MDA-MB-453 breast carcinoma cells.

FIG. 52 is a dose response curve of Compound K, erlotinib and a combination thereof for erlotinib-resistant T47D breast carcinoma cells.

FIG. 53 is a bar graph showing the percent cell death for Compound K, erlotinib and the combination thereof for erlotinib-resistant T47D breast carcinoma cells.

FIG. 54 is a dose response curve of Compound K, erlotinib and a combination thereof for erlotinib-resistant ZR-75-1 breast carcinoma cells.

FIG. 55 is a bar graph showing the percent cell death for Compound K, erlotinib and the combination thereof for erlotinib-resistant ZR-75-1 breast carcinoma cells.

FIG. 56 is a dose response curve of Compound K, Lapatinib and a combination thereof for T47D breast carcinoma cells.

FIG. 57 is a dose response curve of Compound K, sorafenib and a combination thereof for T47D breast carcinoma cells.

FIG. 58 is a bar graph showing the percent cell death for Compound K, sorafenib and the combination thereof for T47D breast carcinoma cells.

FIG. 59 is a dose response curve of Compound K, sunitinib and a combination thereof for T47D breast carcinoma cells.

FIG. 60 is a dose response curve of Compound K, Akt1/2 inhibitor and a combination thereof for BT-474 breast carcinoma cells.

FIG. 61 is a bar graph showing the percent cell death for Compound K, Akt1/2 inhibitor and a combination thereof for BT-474 breast carcinoma cells.

FIG. 62 is a Western blot analysis using the following in the breast carcinoma cell line MDA-MB-453:

### Column 1: Untreated;

### Columns 2, 7, 12: 10 uM Compound K;

### Columns 3, 8, 13: 100 uM Erlotinib;

### Columns 4, 9, 14: 2 uM Lapatinib;

Columns 5, 10, 15: 10 uM Compound K plus 100 uM Erlotinib;
Columns 6, 11, 16: 10 uM Compound K plus 2 uM Lapatinib.

FIG. 63 is a dose response curve of Compound K, panobinostat and a combination thereof for Hs 578T breast cancer cells.

FIG. 64 is a bar graph showing the percent cell death for Compound K, panobinostat and a combination thereof for Hs 578T breast cancer cells.

FIG. 65 is a dose response curve for Compound K, 17-DMAG and a combination thereof for Hs 578T breast cancer cells.

FIG. 66 is a bar graph showing the percent cell death for Compound K, 17-DMAG and a combination thereof for Hs 578T breast cancer cells.

FIG. 67 is a dose response curve for Compound K, AKTi VIII and a combination thereof for BT-474 breast cancer cells.

FIG. 68 is a bar graph showing the percent cell death for Compound K, AKTi VIII and a combination thereof for BT-474 breast cancer cells.

FIG. 69 is a dose response curve for Compound K, BEZ-235 and a combination thereof for BT-474 breast cancer cells.

FIG. 70 is a bar graph showing the percent cell death for Compound K, BEZ-235 and a combination thereof for BT-474 breast cancer cells.

FIG. 71 is a dose response curve for Compound K, LY294002 and a combination thereof for BT-474 breast cancer cells.

FIG. 72 is a bar graph showing the percent cell death for Compound K, LY294002 and a combination thereof for BT-474 breast cancer cells.

FIG. 73 is a dose response curve for Compound K, PI-103 and a combination thereof for BT-474 breast cancer cells.

FIG. 74 is a bar graph showing the percent cell death for Compound K, PI-103 and a combination thereof for BT-474 breast cancer cells.

FIG. 75 is a dose response curve for Compound K, wortmannin and a combination thereof for BT-474 breast cancer cells.

FIG. 76 is a bar graph showing the percent cell death for Compound K, wortmannin and a combination thereof for BT-474 breast cancer cells.

FIG. 77 is a dose response curve for Compound K, PI-103 and a combination thereof for T-47D breast cancer cells.

FIG. 78 is a bar graph showing the percent cell death for Compound K, PI-103 and a combination thereof for T-47D breast cancer cells.

FIG. 79 is a Western hybridization analysis in BT-474 breast cancer cells for the following: untreated cells, cells treated with 5 uM Compound K, with 1 uM AKTi VIII and with 5:1 combination thereof.

FIG. 80 is a graphical representation of the phosphorylation of AKT at S129, at T308, and at 5473, as well as of the cleavage of PARP in BT-474 breast cancer cells for the following: untreated cells, cells treated with 5 uM Compound K, with 1 uM AKTi VIII and with 5:1 combination thereof.

### DETAILED DESCRIPTION

The present application may be understood more readily by reference to the following detailed description of the embodiments and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular forms “a”, “an”, and “the” include plural references unless indicated otherwise.

As used herein, the term “subject” refers to a human or animal subject. Generally, the subject is human.

The term “neoplastic disorder” as used herein refers to a disorder involving aberrant cell proliferation, such as a cancer, for example. The cancer may result in a tumor in certain instances, and symptoms associated with a tumor sometimes are treated. Neoplastic disorders include, but are not limited to, abnormal cell proliferative conditions (e.g., cancer) of the hemopoietic system (e.g., white blood cell), lung, breast, prostate, kidney, pancreas, liver, heart, skeleton, colon, rectum, skin, brain, eye, lymph node, heart, testes or ovary, for example.

The term “therapeutically effective amount” or “effective amount” is intended to mean that amount of a drug or pharmaceutical agent that will elicit a biological or medical response of a cell, tissue, system, animal or human that is being sought by a researcher, veterinarian, medical doctor or other clinician. When referring to the amount of a compound of the application administered in combination with an additional anticancer agent, the “therapeutically effective amount” of the compound of the application may be an amount sufficient to produce an anticancer effect alone, or may be an amount sufficient to produce an anticancer effect in the presence of the additional anticancer agent. Similarly, the amount of the additional anticancer agent may be sufficient to provide an anticancer effect alone, or may be sufficient to provide an anticancer effect in the presence of the compound of the application.

In some embodiments, the combination of a compound of the application and an additional anticancer agent exhibits an additive anticancer effect, such as an additive effect on inhibiting cell proliferation. In other embodiments, the combination of a compound of the application and an additional anticancer agent exhibits a synergistic anticancer effect, such as a synergistic effect on inhibiting cell proliferation.

By “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods and compositions of the present application.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents contain 1-10C (alkyl) or 2-10C (alkenyl or alkynyl). Generally they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C₁-C8 acyl, C2-C8 heteroacyl, C6-C10 aryl, C5-C10 heteroaryl, C7-C12 arylalkyl, or C6-C12 heteroarylalkyl, and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C₄ alkyl, C1-C₄ heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and C(═O)NR₂ as well as C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Often the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Generally, an arylalkyl group includes a phenyl ring optionally substituted with the groups defined above for aryl groups and a C1-C₄ alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group generally includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)— where n is 1-8 and often n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the application, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Generally halo refers to fluoro or chloro.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydroisobenzofuran, isoxazole, 4,5-dihydroisoxazole, piperidine, pyrrolidine, pyrrolidin-2-one, pyrrole, pyridine, pyrimidine, octahydropyrrolo[3,4-b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro thiophene-1,1-dioxide, diazepine, triazole, guanidine, diazabicyclo[2.2.1]heptane, 2,5-diazabicyclo[2.2.1]heptane, 2,3,4,4a,9,9a hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, etc.

The terms “treat”, “treating” or “treatment” in reference to a particular disease or disorder includes prevention of the disease or disorder, and/or lessening, improving, ameliorating or abrogating the symptoms and/or pathology of the disease or disorder. Generally the terms as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety. In certain embodiments, a polar substituent is selected from a carboxy, a carboxy bioisostere or other acid-derived moiety that exists predominately as an anion at a pH of about 7 to 8. Other polar substituents include, but are not limited to, groups containing an OH or NH, an ether oxygen, an amine nitrogen, an oxidized sulfur or nitrogen, a carbonyl, a nitrile, and a nitrogen-containing or oxygen-containing heterocyclic ring whether aromatic or non-aromatic. In some embodiments, the polar substituent represented by R³ is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of:

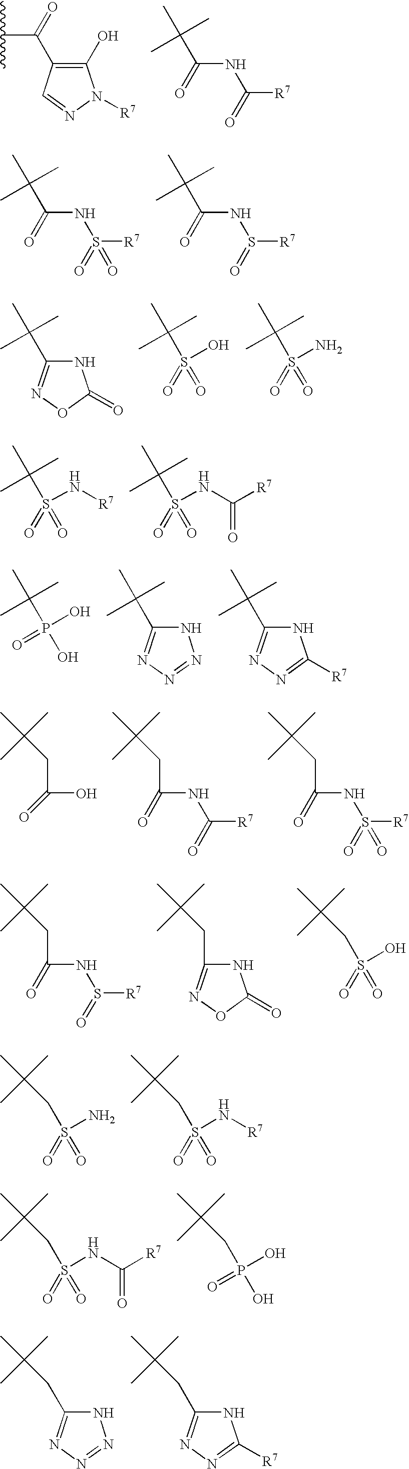

and salts and prodrugs of the foregoing, wherein each R⁷ is independently H or an optionally substituted member selected from the group consisting of C₁₋₁₀ alkyl, C₂₋₁₀ alkenyl, C₁₋₁₀ heteroalkyl, C₃₋₈ carbocyclic ring, and C₃₋₈ heterocyclic ring optionally fused to an additional optionally substituted carbocyclic or heterocyclic ring; or R⁷ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring.

In certain embodiments, the polar substituent is selected from the group consisting of carboxylic acid, carboxylic ester, carboxamide, tetrazole, triazole, carboxymethanesulfonamide, oxadiazole, oxothiadiazole, thiazole, aminothiazole and hydroxythiazole.

In some embodiments, at least one R⁸ present is a carboxylic acid or a salt, or ester or a bioisostere thereof. In certain embodiments, at least one R⁸ present is a carboxylic acid-containing substituent or a salt, ester or bioisostere thereof. In the latter embodiments, the R⁸ substituent may be a C1-C10 alkyl or C1-C10 alkenyl linked to a carboxylic acid (or salt, ester or bioisostere thereof).

### Anticancer Agents

Compounds of the application are administered in combination with an additional anticancer agent, as further described herein. Such additional “anticancer agents” include classic chemotherapeutic agents, as well as molecular targeted therapeutic agents, biologic therapy agents, and radiotherapeutic agents.

Anticancer agents used in combination with the compounds of the present application may include agents selected from any of the classes known to those of ordinary skill in the art, including, for example, alkylating agents, anti-metabolites, plant alkaloids and terpenoids (e.g., taxanes), topoisomerase inhibitors, anti-tumor antibiotics, hormonal therapies, molecular targeted agents, and the like. Generally such an anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, or a PI3K inhibitor.

Alkylating agents include (a) alkylating-like platinum-based chemotherapeutic agents such as cisplatin, carboplatin, nedaplatin, oxaliplatin, satraplatin, and (SP-4-3)-(cis)-amminedichloro-[2-methylpyridine] platinum(II); (b) alkyl sulfonates such as busulfan; (c) ethyleneimine and methylmelamine derivatives such as altretamine and thiotepa; (d) nitrogen mustards such as chlorambucil, cyclophosphamide, estramustine, ifosfamide, mechlorethamine, trofosamide, prednimustine, melphalan, and uramustine; (e) nitrosoureas such as carmustine, lomustine, fotemustine, nimustine, ranimustine and streptozocin; (f) triazenes and imidazotetrazines such as dacarbazine, procarbazine, temozolamide, and temozolomide.

Anti-metabolites include (a) purine analogs such as fludarabine, cladribine, chlorodeoxyadenosine, clofarabine, mercaptopurine, pentostatin, and thioguanine; (b) pyrimidine analogs such as fluorouracil, gemcitabine, capecitabine, cytarabine, azacitidine, edatrexate, floxuridine, and troxacitabine; (c) antifolates, such as methotrexate, pemetrexed, raltitrexed, and trimetrexate. Anti-metabolites also include thymidylate synthase inhibitors, such as fluorouracil, raltitrexed, capecitabine, floxuridine and pemetrexed; and ribonucleotide reductase inhibitors such as claribine, clofarabine and fludarabine.

Plant alkaloid and terpenoid derived agents include mitotic inhibitors such as the vinca alkaloids vinblastine, vincristine, vindesine, and vinorelbine; and microtubule polymer stabilizers such as the taxanes, including, but not limited to paclitaxel, docetaxel, larotaxel, ortataxel, and tesetaxel.

Topoisomerase inhibitors include topoisomerase I inhibitors such as camptothecin, topotecan, irinotecan, rubitecan, and belotecan; and topoisomerase II inhibitors such as etoposide, teniposide, and amsacrine.

Anti-tumor antibiotics include (a) anthracyclines such as daunorubicin (including liposomal daunorubicin), doxorubicin (including liposomal doxorubicin), epirubicin, idarubicin, and valrubicin; (b) streptomyces-related agents such as bleomycin, actinomycin, mithramycin, mitomycin, porfiromycin; and (c) anthracenediones, such as mitoxantrone and pixantrone. Anthracyclines have three mechanisms of action: intercalating between base pairs of the DNA/RNA strand; inhibiting topoiosomerase II enzyme; and creating iron-mediated free oxygen radicals that damage the DNA and cell membranes. Anthracyclines are generally characterized as topoisomerase II inhibitors.

Hormonal therapies include (a) androgens such as fluoxymesterone and testolactone; (b) antiandrogens such as bicalutamide, cyproterone, flutamide, and nilutamide; (c) aromatase inhibitors such as aminoglutethimide, anastrozole, exemestane, formestane, and letrozole; (d) corticosteroids such as dexamethasone and prednisone; (e) estrogens such as diethylstilbestrol; (f) antiestrogens such as fulvestrant, raloxifene, tamoxifen, and toremifine; (g) LHRH agonists and antagonists such as buserelin, goserelin, leuprolide, and triptorelin; (h) progestins such as medroxyprogesterone acetate and megestrol acetate; and (i) thyroid hormones such as levothyroxine and liothyronine.

Molecular targeted agents include (a) receptor tyrosine kinase (‘RTK’) inhibitors, such as inhibitors of EGFR, including erlotinib, gefitinib, and neratinib; inhibitors of VEGFR including vandetanib, semaxinib, and cediranib; and inhibitors of PDGFR; further included are RTK inhibitors that act at multiple receptor sites such as lapatinib, which inhibits both EGFR and HER2, as well as those inhibitors that act at of each of C-kit, PDGFR and VEGFR, including but not limited to axitinib, sunitinib, sorafenib and toceranib; also included are inhibitors of BCR-ABL, c-kit and PDGFR, such as imatinib; (b) FKBP binding agents, such as an immunosuppressive macrolide antibiotic, including bafilomycin, rapamycin (sirolimus) and everolimus; (c) gene therapy agents, antisense therapy agents, and gene expression modulators such as the retinoids and rexinoids, e.g. adapalene, bexarotene, trans-retinoic acid, 9-cis-retinoic acid, and N-(4-hydroxyphenyl)retinamide; (d) phenotype-directed therapy agents, including: monoclonal antibodies such as alemtuzumab, bevacizumab, cetuximab, ibritumomab tiuxetan, rituximab, and trastuzumab; (e) immunotoxins such as gemtuzumab ozogamicin; (f) radioimmunoconjugates such as 131I-tositumomab; and (g) cancer vaccines.

Akt inhibitors include 1L6-Hydroxymethyl-chiro-inositol-2-(R)-2-O-methyl-3-β-octadecyl-sn-glycerocarbonate, SH-5 (Calbiochem Cat. No. 124008), SH-6 (Calbiochem Cat. No. Cat. No. 124009), Calbiochem Cat. No. 124011, Triciribine (NSC 154020, Calbiochem Cat. No. 124012), 10-(4′-(N-diethylamino)butyl)-2-chlorophenoxazine, Cu(II) Cl₂(3-Formylchromone thiosemicarbazone), 1,3-dihydro-1-(1-((4-(6-phenyl-1H-imidazo[4,5-g]quinoxalin-7-yl)phenyl)methyl)-4-piperidinyl)-2H-benzimidazol-2-one, GSK690693 (4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol), SR13668 ((2,10-dicarbethoxy-6-methoxy-5,7-dihydro-indolo[2,3-b]carbazole), GSK2141795, Perifosine, GSK21110183, XL418, XL147, PF-04691502, BEZ-235 [2-Methyl-244-(3-methyl-2-oxo-8-quinolin-3-yl-2,3-dihydro-imidazo[4,5-c]quinolin-1-yl)-phenyl]-propionitrile], PX-866 ((acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-1′-yl ester)), D-106669, CAL-101, GDC0941 (2-(1H-indazol-4-yl)-6-(4-methanesulfonyl-piperazin-1-ylmethyl)-4-morpholin-4-yl-thieno[3,2-d]pyrimidine), SF1126, SF1188, SF2523, TG100-115 [3-[2,4-d]amino-6-(3-hydroxyphenyl)pteridin-7-yl]phenol]. A number of these inhibitors, such as, for example, BEZ-235, PX-866, D 106669, CAL-101, GDC0941, SF1126, SF2523 are also identified in the art as PI3K/mTOR inhibitors; additional examples, such as PI-103 [3-[4-(4-morpholinylpyrido[3′,2′:4,5]furo[3,2-d]pyrimidin-2-yl]phenol hydrochloride] are well-known to those of skill in the art. Additional well-known PI3K inhibitors include LY294002 [2-(4-morpholinyl)-8-phenyl-4H-1-benzopyran-4-one] and wortmannin. mTOR inhibitors known to those of skill in the art include temsirolimus, deforolimus, sirolimus, everolimus, zotarolimus, and biolimus A9. A representative subset of such inhibitors includes temsirolimus, deforolimus, zotarolimus, and biolimus A9.

HDAC inhibitors include (i) hydroxamic acids such as Trichostatin A, vorinostat (suberoylanilide hydroxamic acid (SAHA)), panobinostat (LBH589) and belinostat (PXD101) (ii) cyclic peptides, such as trapoxin B, and depsipeptides, such as romidepsin (NSC 630176), (iii) benzamides, such as MS-275 (3-pyridylmethyl-N-{4-[(2-aminophenyl)-carbamoyl]-benzyl}-carbamate), CI994 (4-acetylamino-N-(2-aminophenyl)-benzamide) and MGCD0103 (N-(2-aminophenyl)-4-((4-(pyridin-3-yl)pyrimidin-2-ylamino)methyl)benzamide), (iv) electrophilic ketones, (v) the aliphatic acid compounds such as phenylbutyrate and valproic acid.

Hsp90 inhibitors include benzoquinone ansamycins such as geldanamycin, 17-DMAG (17-Dimethylamino-ethylamino-17-demethoxygeldanamycin), tanespimycin (17-AAG, 17-allylamino-17-demethoxygeldanamycin), EC5, retaspimycin (IPI-504, 18,21-didehydro-17-demethoxy-18,21-dideoxo-18,21-dihydroxy-17-(2-propenylamino)-geldanamycin), and herbimycin; pyrazoles such as CCT 018159 (4-[4-(2,3-dihydro-1,4-benzodioxin-6-yl)-5-methyl-1H-pyrazol-3-yl]-6-ethyl-1,3-benzenediol); macrolides, such as radicocol; as well as BIIB021 (CNF2024), SNX-5422, STA-9090, and AUY922.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-α2a and interferon-α2b, and interleukins such as aldesleukin, denileukin diftitox, and oprelvekin.

In addition to anticancer agents intended to act against cancer cells, combination therapies including the use of protective or adjunctive agents, including: cytoprotective agents such as armifostine, dexrazonxane, and mesna, phosphonates such as pamidronate and zoledronic acid, and stimulating factors such as epoetin, darbeopetin, filgrastim, PEG-filgrastim, and sargramostim, are also envisioned.

In one aspect, the application discloses a method for treating or ameliorating a neoplastic disorder, comprising administering to a subject in need thereof a therapeutically effective amount of a compound of Formula I:

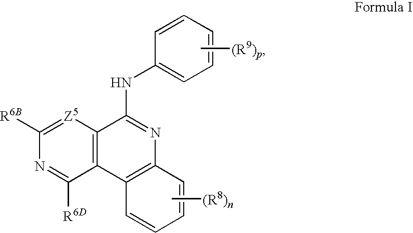

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4;

and an anticancer agent, or a pharmaceutically acceptable salt or ester thereof;

thereby treating or ameliorating said neoplastic disorder.

In one alternative, the application discloses a method for treating or ameliorating a neoplastic disorder, comprising administering to a subject in need thereof a therapeutically effective amount of a compound of Formula I as described herein and an anticancer agent, or a pharmaceutically acceptable salt or ester thereof, wherein the anticancer agent is not doxorubicin.

In another alternative, the application discloses a method for treating or ameliorating a neoplastic disorder, comprising administering to a subject in need thereof a therapeutically effective amount of a compound of Formula I as described herein and an anticancer agent, or a pharmaceutically acceptable salt or ester thereof, wherein the anticancer agent is not a topoisomerase II inhibitor.

In still another alternative, the application discloses a method for treating or ameliorating a neoplastic disorder, comprising administering to a subject in need thereof a therapeutically effective amount of a compound of Formula I as described herein and an anticancer agent, or a pharmaceutically acceptable salt or ester thereof, wherein the anticancer agent is not an anti-tumor antibiotic.

In one embodiment of any of aspect or alternative described herein, the anticancer agent is not 5-fluorouracil. In another embodiment, the anticancer agent is not a thymidylate synthase inhibitor. In yet another embodiment, the anticancer agent is not an antimetabolite pyrmidine analog. In still another embodiment, the anticancer agent is not an antimetabolite.

In one embodiment of any of aspect or alternative described herein, the anticancer agent is not rapamycin. In another embodiment, the anticancer agent is not an immunosuppressive macrolide antibiotic. In yet another embodiment, the anticancer agent is not FKBP binding agent.

In one embodiment of any of aspect or alternative described herein, the anticancer agent is not erlotinib (Tarceva). In another embodiment, the anticancer agent is not a small molecule EGFR inhibitor. In yet another embodiment, the anticancer agent is not a receptor tyrosine kinase inhibitor.

In one embodiment of any of aspect or alternative described herein, the anticancer agent is not sunitinib (Sutent). In another embodiment, the anticancer agent is not an inhibitor of VEGFR, PDGFR and cKIT. In yet another embodiment, the anticancer agent is not a receptor tyrosine kinase inhibitor.

In another embodiment of any aspect or alternative described herein, the anticancer agent is not doxorubicin, 5-fluorouracil, rapamycin, erlotinib or sunitinib. In another embodiment, the anticancer agent is not any one of any four of doxorubicin, 5-fluorouracil, rapamycin, erlotinib or sunitinib. For example in one such embodiment, the anticancer agent is not 5-fluorouracil, rapamycin, erlotinib or sunitinib. In another such embodiment the anticancer agent is not doxorubicin, 5-fluorouracil, erlotinib or sunitinib. In another embodiment, the anticancer agent is not any one of any three of doxorubicin, 5-fluorouracil, rapamycin, erlotinib or sunitinib. For example in one such embodiment, the anticancer agent is not 5-fluorouracil, erlotinib or sunitinib. In another such embodiment the anticancer agent is not doxorubicin, erlotinib or sunitinib. In another embodiment, the anticancer agent is not any one of any two of doxorubicin, 5-fluorouracil, rapamycin, erlotinib or sunitinib.

In another embodiment of any aspect or alternative described herein, the anticancer agent is not a topoisomerase II inhibitor, a thymidylate synthase inhibitor, an immunosuppressive macrolide antibiotic, a small molecule EGFR inhibitor or an inhibitor of VEGFR, PDGFR and cKIT. In another embodiment, the anticancer agent is not any one of any four of a topoisomerase II inhibitor, a thymidylate synthase inhibitor, an immunosuppressive macrolide antibiotic, a small molecule EGFR inhibitor or an inhibitor of VEGFR, PDGFR and cKIT. For example in one such embodiment, the anticancer agent is not a thymidylate synthase inhibitor, an immunosuppressive macrolide antibiotic, a small molecule EGFR inhibitor or an inhibitor of VEGFR, PDGFR and cKIT. In another such embodiment the anticancer agent is not a topoisomerase II inhibitor, thymidylate synthase inhibitor, a small molecule EGFR inhibitor or an inhibitor of VEGFR, PDGFR and cKIT. In another embodiment, the anticancer agent is not any one of any three of a topoisomerase II inhibitor, thymidylate synthase inhibitor, an immunosuppressive macrolide antibiotic, a small molecule EGFR inhibitor or an inhibitor of VEGFR, PDGFR and cKIT. For example in one such embodiment, the anticancer agent is not a topoisomerase II inhibitor, thymidylate synthase inhibitor, or an inhibitor of VEGFR, PDGFR and cKIT. In another such embodiment the anticancer agent is not a thymidylate synthase inhibitor, a small molecule EGFR inhibitor or an inhibitor of VEGFR, PDGFR and cKIT. In another embodiment, the anticancer agent is not any one of any two of a topoisomerase II inhibitor, thymidylate synthase inhibitor, an immunosuppressive macrolide antibiotic, a small molecule EGFR inhibitor or an inhibitor of VEGFR, PDGFR and cKIT.

In another embodiment of any aspect or alternative described herein, the anticancer agent is not a topoisomerase II inhibitor, an antimetabolite pyrimidine analog, an FKBP binding agent, or a receptor tyrosine kinase inhibitor. In another embodiment, the anticancer agent is not any one of any three of a topoisomerase II inhibitor, an antimetabolite pyrimidine analog, an FKBP binding agent, or a receptor tyrosine kinase inhibitor. For example in one such embodiment, the anticancer agent is not a topoisomerase II inhibitor, an antimetabolite pyrimidine analog, or a receptor tyrosine kinase inhibitor. In another such embodiment the anticancer agent is not an antimetabolite pyrimidine analog, an FKBP binding agent, or a receptor tyrosine kinase inhibitor. In another embodiment, the anticancer agent is not any one of any two of a topoisomerase II inhibitor, an antimetabolite pyrimidine analog, an FKBP binding agent, or a receptor tyrosine kinase inhibitor.

In one embodiment of any aspect or alternative described herein, the anticancer agent used in combination with a compound of the present application is selected from 5-fluorouracil (5-FU), cisplatin, doxorubicin, fludarabine, gemcitabine, paclitaxel, rapamycin, sunitinib, lapatinib, sorafenib, erlotinib, and vinblastine. In one embodiment of any aspect or alternative described herein, the anticancer agent used in combination with a compound of the present application is selected from 5-fluorouracil (5-FU), cisplatin, doxorubicin, fludarabine, gemcitabine, paclitaxel, rapamycin, sunitinib, erlotinib, and vinblastine. In another embodiment, the anticancer agent is selected from 5-fluorouracil, cisplatin, fludarabine, gemcitabine, paclitaxel, rapamycin, sunitinib, erlotinib, and vinblastine. In yet another embodiment, the anticancer agent is selected from cisplatin, doxorubicin, fludarabine, gemcitabine, paclitaxel, rapamycin, sunitinib, erlotinib, and vinblastine. In still another embodiment, the anticancer agent is selected from cisplatin, fludarabine, gemcitabine, paclitaxel, rapamycin, sunitinib, erlotinib, and vinblastine. In another embodiment, the anticancer agent is selected from 5-fluorouracil, cisplatin, doxorubicin, fludarabine, gemcitabine, paclitaxel, sunitinib, erlotinib, and vinblastine. In yet another embodiment, the anticancer agent is selected from 5-fluorouracil, cisplatin, fludarabine, gemcitabine, paclitaxel, sunitinib, erlotinib, and vinblastine. In still another embodiment, the anticancer agent is selected from 5-fluorouracil, cisplatin, doxorubicin, fludarabine, gemcitabine, paclitaxel, rapamycin, sunitinib, and vinblastine. In a further embodiment, the anticancer agent is selected from 5-fluorouracil, cisplatin, fludarabine, gemcitabine, paclitaxel, rapamycin, sunitinib, and vinblastine. In an additional embodiment, the anticancer agent is selected from 5-fluorouracil, cisplatin, doxorubicin, fludarabine, gemcitabine, paclitaxel, rapamycin, erlotinib, and vinblastine. In another embodiment, the anticancer agent is selected from 5-fluorouracil, cisplatin, fludarabine, gemcitabine, paclitaxel, rapamycin, erlotinib, and vinblastine. In yet another embodiment, the anticancer agent used in combination with a compound of the present application is selected from doxorubicin, cisplatin, fludarabine, gemcitabine, paclitaxel, and vinblastine. In still another embodiment, the anticancer agent used in combination with a compound of the present application is selected from cisplatin, fludarabine, gemcitabine, paclitaxel, and vinblastine. In yet another embodiment, the anticancer agent used in combination with a compound of the present application is selected from sunitinib, lapatinib, sorafenib and erlotinib.

In another embodiment of any disclosed aspect or alternative described herein, the anticancer agent used in combination with a compound of the present application is selected from an Akt inhibitor, an HDAC inhibitor, an Hsp90 inhibitor, an mTOR inhibitor, a PI3K/mTOR inhibitor, and a PI3K inhibitor. In one embodiment, the anticancer agent used in combination with a compound of the present application is selected from an inhibitor of Akt1/2, an hydroxamic acid inhibitor of HDAC, and a benzoquinone ansamycin inhibitor of Hsp90. In another embodiment, the anticancer agent used in combination with a compound of the present invention is selected from 1,3-dihydro-1-(1-((4-(6-phenyl-1H-imidazo[4,5-g]quinoxalin-7-yl)phenyl)methyl)-4-piperidinyl)-2H-benzimidazol-2-one, panobinostat and 17-DMAG. In another embodiment, the anticancer agent used in combination with a compound of the present application is selected from an imidazo[4,5-c]quinoline derivative that inhibits PI3K and mTOR kinase activity, a benzopyran derivative that inhibits PI3K, a pyrido[3′,2′:4,5]furo[3,2-d]pyrimidine derivative that inhibits PI3K and mTOR kinase activity and a furanosteroid derivative that inhibits PI3K. In yet another embodiment, the anticancer agent used in combination with a compound of the present invention is selected from BEZ-235, LY294002, PI-103, and wortmannin.

In one embodiment of any disclosed aspect or alternative, the compound of Formula I has the structure of Formula II, III, IV, V or VI:

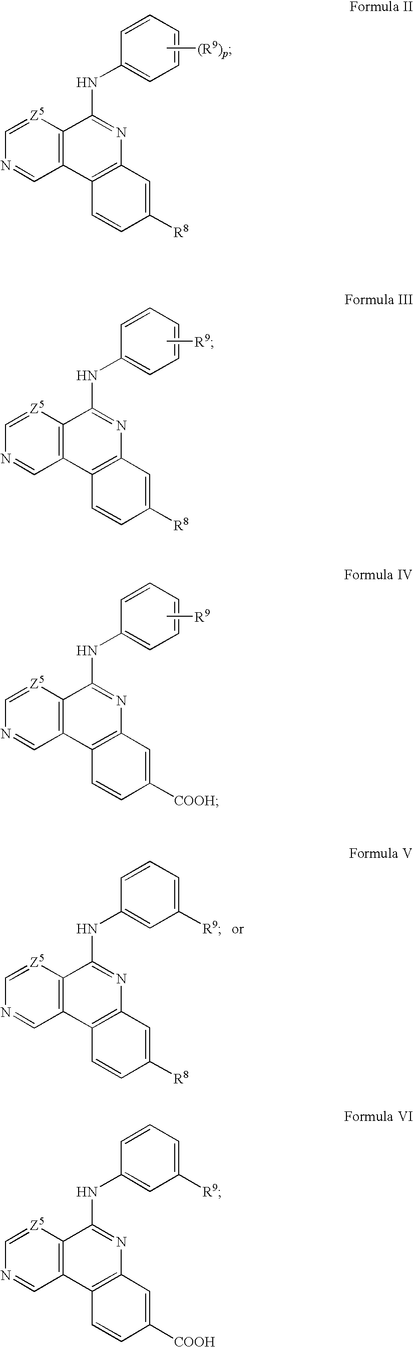

or a pharmaceutically acceptable salt or ester thereof;

wherein Z⁵ is N or CR^{6A};

each R^{6A} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S; and

p is 0 to 4.

In one embodiment of any disclosed aspect or alternative, the compound of Formula I has the structure of Formula II. In another embodiment, the compound of Formula I has the structure of Formula III. In yet another embodiment, the compound of Formula I has the structure of Formula IV. In still a further embodiment, the compound of Formula I has the structure of Formula V. In yet another embodiment of any disclosed aspect or alternative, the compound of Formula I has the structure of Formula VI. In one variation of any disclosed embodiment, Z⁵ is CR^{6A}. In one particular variation of any disclosed embodiment, Z⁵ is CH.

In a particular embodiment of any disclosed aspect or alternative, the compound of Formula I is a compound (Compound K) having the formula:

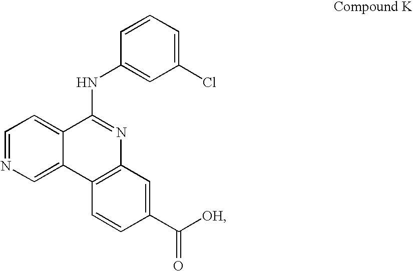

or a pharmaceutically acceptable salt or ester thereof.

In another embodiment of any disclosed aspect or alternative, the compound of formula I is a compound having formula (1) or (2):

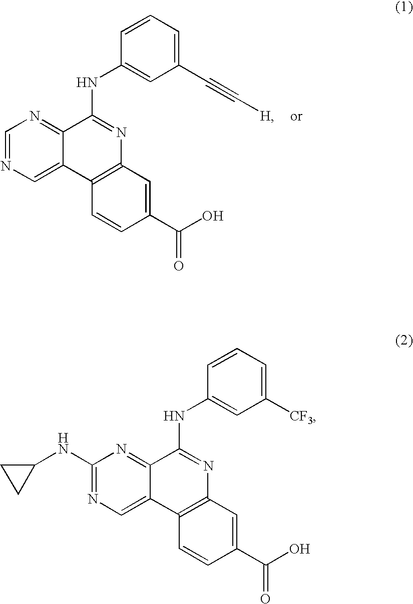

or a pharmaceutically acceptable salt or ester thereof.

Compounds of Formulae I, II, III, IV, V, and VI can exert biological activities that include, but are not limited to, inhibiting cell proliferation and modulating protein kinase activity. Compounds of such Formulae can modulate CK2 activity, for example. Such compounds therefore can be utilized in multiple applications by a person of ordinary skill in the art. For example, compounds described herein may find uses that include, but are not limited to, (i) modulation of protein kinase activity (e.g., CK2 activity), (ii) modulation of cell proliferation, (iii) modulation of apoptosis, and (iv) treatment of cell proliferation related disorders, such as neoplastic disorders, when administered alone or in combination with another anticancer agent.

In another aspect, the application discloses a method for inhibiting or slowing cell proliferation in a system, comprising administering to said system an effective amount of a compound of Formula I, II, III, IV, V, or VI, as described herein, or a pharmaceutically acceptable salt or ester thereof, and an anticancer agent or a pharmaceutically acceptable salt or ester thereof; thereby inhibiting or slowing cell proliferation. The system may be a cell, tissue or subject.

The present application also discloses methods for preventing, treating or ameliorating neoplastic disorders, as well as for inhibiting or slowing cell proliferation, comprising the administration of a therapeutically effective amount of a compound (Compound K) having the formula:

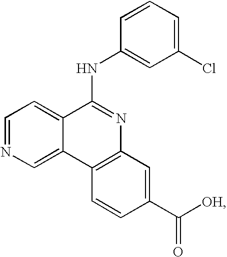

or a pharmaceutically acceptable salt or ester thereof, in combination with commonly used anticancer agents, or pharmaceutically acceptable salts or esters thereof.

With regard to the foregoing aspects of the application, the inventors contemplate any combination of the anticancer agents as set forth herein.

The present application discloses pharmaceutical compositions comprising a compound of Formula I, II, III, IV, V or VI, or a pharmaceutically acceptable salt or ester thereof, and a commonly used anticancer agent, or a pharmaceutically acceptable salt or ester thereof, and at least one pharmaceutically acceptable excipient. The combination is administered in an amount effective to inhibit cell proliferation.

Compounds of Formula I, II, III, IV, V and VI, and the pharmaceutically acceptable salts and esters thereof, are sometimes collectively referred to herein as compounds of the application.

The present application further discloses pharmaceutical compositions comprising a compound of the application or a pharmaceutically acceptable salt or ester thereof, and a commonly used anticancer agent, or a pharmaceutically acceptable salt or ester thereof, and at least one pharmaceutically acceptable excipient. The combination is administered in an amount effective to inhibit cell proliferation. In specific embodiments, the compound of the application is Compound K, Compound 1, or Compound 2, or a salt or ester thereof.

In one aspect disclosed in the present application, the combination therapy is administered to individuals who have a neoplastic disorder. In another aspect of the present application, the combination therapy is administered to individuals who do not yet show clinical signs of a neoplastic disorder, but who are at risk of developing a neoplastic disorder. Toward this end, the present application discloses methods for preventing or reducing the risk of developing a neoplastic disorder.

In one embodiment, a single pharmaceutical dosage formulation that contains both a compound of the application, such as Compound K, and the anticancer agent is administered. In another embodiment disclosed in the application, separate dosage formulations are administered; the compound and the anticancer agent may be, for example, administered at essentially the same time, for example, concurrently, or at separately staggered times, for example, sequentially. In certain examples, the individual components of the combination may be administered separately, at different times during the course of therapy, or concurrently, in divided or single combination forms.

The present application discloses, for example, simultaneous, staggered, or alternating treatment. Thus, the compound of the application may be administered at the same time as an anticancer agent, in the same pharmaceutical composition; the compound of the application may be administered at the same time as the anticancer agent, in separate pharmaceutical compositions; the compound of the application may be administered before the anticancer agent, or the anticancer agent may be administered before the compound of the application, for example, with a time difference of seconds, minutes, hours, days, or weeks. In examples of a staggered treatment, a course of therapy with the compound of the application may be administered, followed by a course of therapy with the anticancer agent, or the reverse order of treatment may be used, more than one series of treatments with each component may be used. In certain examples of the present application, one component, for example, the compound of the application or the anticancer agent, is administered to a mammal while the other component, or its derivative products, remains in the bloodstream of the mammal. For example, Compound K may be administered while the anticancer agent or its derivative products remains in the bloodstream, or the anticancer agent may be administered while Compound K or its derivatives remains in the bloodstream. In other examples, the second component is administered after all, or most of the first component, or its derivatives, have left the bloodstream of the mammal.

Anticancer agents used in combination with the compounds of the present application may include agents selected from any of the classes known to those of ordinary skill in the art. Appropriate anticancer agents can include, but are not limited to alkylating agents, anti-metabolites (e.g., purine and pyrimidine agents), plant alkaloids (e.g., vinca alkaloids) terpenoids (e.g., taxanes), topoisomerase inhibitors, anti-tumor antibiotics, hormonal therapies, and molecular targeted agents, such as receptor tyrosine kinase (RTK) inhibitors (e.g., PDGFR, VEFGR, EGFR inhibitors) and monoclonal antibodies, among others.

### Formulation and Administration

While the compositions and methods of the present application will typically be used in therapy for human patients, they may also be used in veterinary medicine to treat similar or identical diseases. The compositions may, for example, be used to treat mammals, including, but not limited to, primates and domesticated mammals. The compositions may, for example be used to treat herbivores. The compositions of the present application include geometric and optical isomers of one or more of the drugs, wherein each drug is a racemic mixture of isomers or one or more purified isomers.

Pharmaceutical compositions suitable for use in the present application include compositions wherein the active ingredients are contained in an effective amount to achieve the intended purpose. Determination of the effective amounts is well within the capability of those skilled in the art, especially in light of the detailed disclosure provided herein.

The compounds of the present application may exist as pharmaceutically acceptable salts. The term “pharmaceutically acceptable salts” is meant to include salts of active compounds which are prepared with relatively nontoxic acids or bases, depending on the particular substituent moieties found on the compounds described herein. When compounds of the present application contain relatively acidic functionalities, base addition salts can be obtained by contacting the neutral form of such compounds with a sufficient amount of the desired base, either neat or in a suitable inert solvent. Included are base addition salts such as sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids, for example, acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present application contain both basic and acidic functionalities that allow the compounds to be converted into either base or acid addition salts.

Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof, including racemic mixtures), succinates, benzoates and salts with amino acids such as glutamic acid. These salts may be prepared by methods known to those skilled in art.

The neutral forms of the compounds are typically regenerated by contacting the salt with a base or acid and isolating the parent compound in the conventional manner. The parent form of the compound differs from the various salt forms in certain physical properties, such as solubility in polar solvents.

The pharmaceutically acceptable esters in the present application refer to non-toxic esters, generally the alkyl esters are methyl, ethyl, propyl, isopropyl, butyl, isobutyl or pentyl esters, more often the alkyl ester is methyl ester. However, other esters such as phenyl-C₁₋₅ alkyl may be employed if desired. Ester derivatives of certain compounds may act as prodrugs which, when absorbed into the bloodstream of a warm-blooded animal, may cleave in such a manner as to release the drug form and permit the drug to afford improved therapeutic efficacy.

Certain compounds of the present application can exist in unsolvated forms as well as solvated forms, including hydrated forms. In general, the solvated forms are equivalent to unsolvated forms and are encompassed within the scope of the present application. Certain compounds of the present application may exist in multiple crystalline or amorphous forms. In general, all physical forms are equivalent for the uses contemplated by the present application and are intended to be within the scope of the present application.

When used as a therapeutic the compounds described herein often are administered with a physiologically acceptable carrier. A physiologically acceptable carrier is a formulation to which the compound can be added to dissolve it or otherwise facilitate its administration. Examples of physiologically acceptable carriers include, but are not limited to, water, saline, physiologically buffered saline.

Certain compounds of the present application possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present application. Therefore, single stereochemical isomers as well as enantiomeric and diastereomeric mixtures of the present compounds are within the scope of the application. The compounds of the present application do not include those which are known in art to be too unstable to synthesize and/or isolate. The present application discloses compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another. It will be apparent to one skilled in the art that certain compounds of this application may exist in tautomeric forms, all such tautomeric forms of the compounds being within the scope of the application.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon are within the scope of this application. The compounds of the present application may also contain unnatural proportions of atomic isotopes at one or more of atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present application, whether radioactive or not, are encompassed within the scope of the present disclosure.

In addition to salt forms, the present application provides compounds that are in a prodrug form. Prodrugs of the compounds described herein are those compounds that readily undergo chemical changes under physiological conditions to provide the compounds of the present application. Additionally, prodrugs can be converted to the compounds of the present application by chemical or biochemical methods in an ex vivo environment. For example, prodrugs can be slowly converted to the compounds of the present application when placed in a transdermal patch reservoir with a suitable enzyme or chemical reagent.

The descriptions of compounds of the present application are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A compound of the present application can be formulated as a pharmaceutical composition. Such a pharmaceutical composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such, as transdermal patches or iontophoresis devices. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975. Other examples of drug formulations can be found in Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable dilutent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Suppositories for rectal administration of the drug can be prepared by mixing the drug with a suitable nonirritating excipient such as cocoa butter, synthetic mono- di- or triglycerides, fatty acids and polyethylene glycols that are sold at ordinary temperatures but liquid at the rectal temperature and will therefore melt in the rectum and release the drug.

Solid dosage forms for oral administration can include capsules, tablets, pills, powders, and granules. In such solid dosage forms, the compounds of this application are ordinarily combined with one or more adjuvants appropriate to the indicated route of administration. If administered per os, a contemplated aromatic sulfone hydroximate inhibitor compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated aromatic sulfone hydroximate inhibitor compound can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form varies depending upon the mammalian host treated and the particular mode of administration.

The dosage regimen utilizing the compounds of the present application in combination with an anticancer agent is selected in accordance with a variety of factors including type, species, age, weight, sex and medical condition of the patient; the severity of the condition to be treated; the route of administration; the renal and hepatic function of the patient; and the particular compound or salt or ester thereof employed. A consideration of these factors is well within the purview of the ordinarily skilled clinician for the purpose of determining the therapeutically effective dosage amounts to be given to a person in need of the instant combination therapy.

### EXAMPLES

The examples set forth below illustrate but do not limit the disclosure.

### Example 1

### Cell Inhibition Assays

Three-thousand (3000) cells are plated per well in each well of two 96 well plates (duplicates). Cells are incubated overnight at 37 degrees C. The following day, one or more of the compounds are added to the plates, and concentrations of each of the compounds are systematically varied across the plates. Typically, one compound is varied vertically using two, three or four-fold dilutions and the second compound is varied horizontally using two, three or four-fold dilutions across each plate (shown hereafter). The top concentration for Compound K is 100, 30 or 10 micromolar. The top concentration for other drugs, such as rapamycin or cisplatin, varies between 200 micromolar and 30 nanomolar. In some cases observed synergy is affected by the order of addition of the two compounds. In these cases the first drug was added one day prior to the second. The analysis is performed with Alamar Blue cell viability. In short, twenty microliters of AlamarBlue reagent (Invitrogen, Carlsbad Calif.) was added per well. The plates were incubated for four hours at 37 degrees Celsius and the resulting fluorescence was measured at Ex 560 nm/Em 590 nm.

### Example 1a

### Calculating IC50s for Single Agents

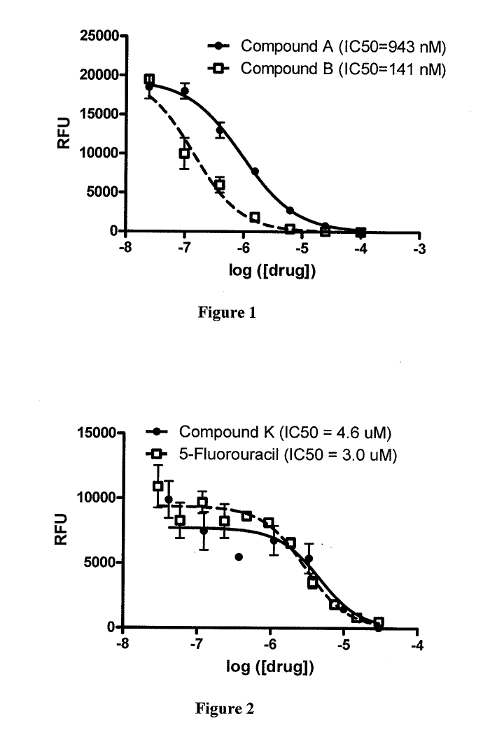

To determine IC50s for single agents for each combination, the duplicates of the raw data in Relative Fluorescent Units (RFU) from Alamar Blue Assay were corrected for background and analyzed with Sigmoidal dose-response (variable slope) using GraphPad Prism Software (GraphPad, San Diego Calif.). The following constrains were applied: Bottom was fixed at equal to zero; in cases where calculated Top was unreasonably high its value was fixed at less or equal to the highest value that was observed in the analyzed data set. See FIG. 1.

### Example 2

### Calculating Synergy from a Plate of Percent Inhibition Data

A percent inhibition is calculated for every well in the plate based on the response data gathered as stated in Example 1. The concentration of Compound K increases regularly as the row number increases from 1 to 8. The high concentration (e.g. 100 micromolar) is serially diluted (e.g. three-fold). The concentration of drug increases regularly as the column letter increases from A to L (as noted in the table below). The high concentration (e.g. 30 micromolar) is serially diluted (e.g. three-fold). A representative plate utilized for the studies is shown hereafter.

| Conc. | | Conc. Drug μM | | | | | | | | | | | |
|---|---|---|---|---|---|---|---|---|---|---|---|---|---|
| Compound | | 0 | 0.0005 | 0.0015 | 0.0046 | 0.014 | 0.041 | 0.12 | 0.37 | 1.1 | 3.3 | 10 | 30 |
| K μM | | A | B | C | D | E | F | G | H | I | J | K | L |
| 0 | 1 | | | | | | | | | | | | |
| 0.14 | 2 | | | | | | | | | | | | |
| 0.41 | 3 | | | | | | | | | | | | |
| 1.2 | 4 | | | | | | | | | | | | |
| 3.7 | 5 | | | | | | | | | | | | |
| 11 | 6 | | | | | | | | | | | | |
| 33 | 7 | | | | | | | | | | | | |
| 100 | 8 | | | | | | | | | | | | |

The expected percent inhibition value is derived by assuming exact additivity between the effect of Compound K and the added drug. Hence the expected value for any well of interest is calculated as the percent inhibition observed for Compound K alone at the same concentration present in that well multiplied by the percent inhibition observed for the added drug alone at the same concentration present in that well. In practice this means the percent inhibition observed for Compound K comes from column A as the concentration of the added drug is 0 here. Similarly, the percent inhibition observed for added drug comes from row 2 (as the concentration of Compound K is 0 here) e.g. the expected value for well D8 is obtained by multiplying the percent inhibition observed in well A8 by the percent inhibition observed in well D2.

Controls for these studies are the dose response curves for each of the two drugs by themselves. Such controls allow one to predict the cytotoxicity for each possible combination for each of the two drugs based simply on adding the cytotoxicity observed for each of the two drugs when used alone.

Assessment of synergy is completed by comparing the actual percent inhibition to the expected percent inhibition. If the expected value for well D8 is 60% but 80% inhibition is observed, the compounds are enhancing each other's effect and synergy is observed, for example. The number shown in the table will be 20.0. Conversely, a negative number is obtained when the two compounds produce less than the expected inhibitory effect.

For example, if concentration X of compound A inhibits by 20%, and concentration Y of compound B inhibits by 20%, one could expect a combination of concentration X of compound A and concentration Y of compound B to inhibit by 40%. That leaves another 60% inhibition possible. For example an overall inhibition of 70% corresponds to 50% inhibition of the remaining 60%, showing as a “50” for that particular combination. In practice, a program is written in the PilotScript programming language to calculate the quantities outlined above.

### Example 2a

### Calculating Synergy Using Combination Index

Combination index (CI) provides quantitative measure of the extent of drug interactions CI=[A]/IC50_{A}+[B]/IC50_{B}, where IC50_{A} and IC50_{B} concentrations of singe agents to achieve 50% effect alone and [A] and [B] concentrations of these two agents to achieve 50% effect in combination. A CI of less than, equal to, and more than 1 indicates synergy, additivity, and antagonism respectively. To calculate CI for our combinations we used IC50s that were determined with Sigmoidal dose-response (variable slope) using GraphPad Prism Software. The value of 50% effect was calculated as a half of an average between the Top value for compound K and combination compound. CI value is calculated at the lowest drug concentrations at which the 50% effect was achieved.

### Example 3

### 5-Fluorouracil/Compound K Combination Testing in A375 Melanoma Cells

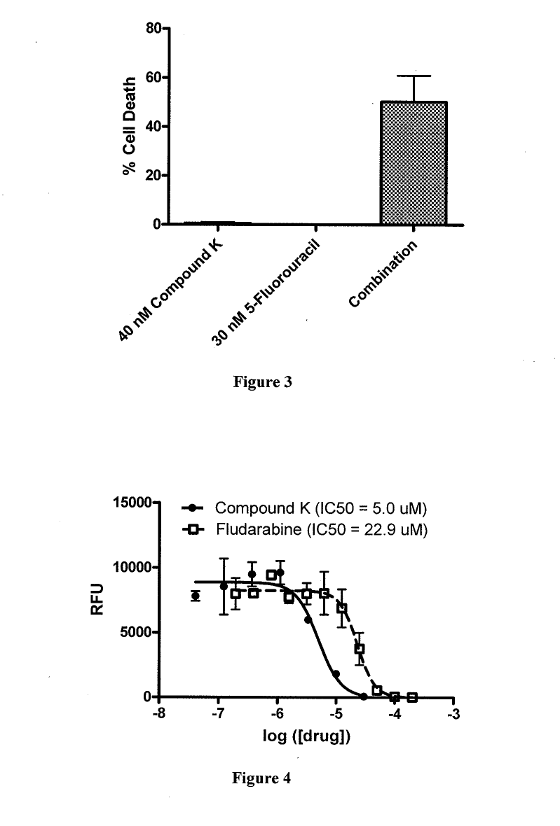

5-Fluorouracil, thymidylate synthase inhibitor, was tested in combination with Compound K in the melanoma cell line A375. 5-Fluorouracil was added 24 hours before Compound K in a 5 day assay. Results are shown hereafter; see FIG. 2 and FIG. 3. Synergy up to 55% is observed at concentrations tested. CI=0.02.

5-Fluorouracil was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.03 | 0.06 | 0.12 | 0.23 | 0.47 | 0.94 | 1.88 | 3.75 | 7.50 | 15.00 | 30 |
| 0 | 0.0 | 0.0 | 0.0 | −0.0 | 0.0 | 0.0 | 0.0 | 0.0 | 0.0 | 0.0 | 0.0 | 0.0 |
| 0.04 | 0.0 | 51.0 | 62.4 | 58.7 | 62.9 | 56.4 | 53.8 | 39.2 | 17.9 | 10.2 | 4.4 | 2.2 |
| 0.12 | 0.0 | 30.8 | 37.6 | 33.1 | 41.6 | 33.5 | 36.3 | 22.6 | 11.3 | 6.0 | 2.9 | 1.1 |
| 0.37 | 0.0 | 30.3 | 35.0 | 34.9 | 39.8 | 37.5 | 33.3 | 22.9 | 10.6 | 6.1 | 2.8 | 0.7 |
| 1.11 | 0.0 | 35.5 | 39.3 | 35.6 | 39.0 | 36.8 | 34.9 | 26.3 | 11.7 | 5.6 | 2.4 | 0.4 |
| 3.33 | 0.0 | 26.7 | 29.0 | 25.2 | 29.1 | 24.4 | 24.4 | 15.6 | 7.4 | 4.0 | 1.5 | −0.6 |
| 10.00 | −0.0 | 8.7 | 9.4 | 8.6 | 9.8 | 8.9 | 8.3 | 5.4 | 1.7 | 0.4 | −0.4 | −1.4 |
| 30 | 0.0 | 0.5 | 0.3 | 0.4 | 0.4 | 0.3 | 0.1 | −0.1 | −0.4 | −0.6 | −0.6 | −1.5 |
| 0 | 0.0 | 0.0 | 0.0 | 0.0 | −0.0 | 0.0 | 0.0 | −0.0 | 0.0 | 0.0 | 0.0 | 0.0 |
| 0.04 | 0.0 | 48.9 | 27.4 | 50.4 | 30.6 | 46.3 | 38.7 | 33.1 | 18.3 | 9.5 | 3.6 | 0.7 |
| 0.12 | 0.0 | 52.6 | 20.8 | 44.6 | 23.1 | 33.0 | 30.0 | 24.4 | 15.6 | 7.8 | 3.0 | 0.9 |
| 0.37 | 0.0 | 23.8 | 6.1 | 19.7 | 8.8 | 11.4 | 11.1 | 11.8 | 6.5 | 3.0 | 0.1 | −3.3 |
| 1.11 | −0.0 | 37.9 | 17.4 | 32.7 | 18.1 | 25.1 | 25.8 | 18.8 | 10.2 | 5.5 | 1.0 | −2.0 |
| 3.33 | 0.0 | 33.3 | 17.4 | 29.2 | 17.2 | 15.9 | 19.1 | 12.6 | 5.3 | 2.4 | −0.1 | −2.5 |
| 10.00 | −0.0 | 5.7 | 1.2 | 5.4 | 2.0 | 3.1 | 3.5 | 2.7 | 1.0 | 0.1 | −0.7 | −2.1 |
| 30 | 0.0 | 0.1 | −0.1 | 0.3 | 0.1 | 0.1 | −0.4 | −0.2 | −0.5 | −0.5 | −0.5 | −0.4 |

Compound K: IC50=4.6 uM, Top=7711 RFU

5-FU: IC50=3.0 uM, Top=9383 RFU

Value of 50% effect=4274 RFU

50% Effect was achieved by combining 40 nM Compound K and 30 nM 5-Fluorouracil.

CI=[Compound K]/IC50_{Compound K}+[5-FU]/IC50_{5-FU}=(0.04/4.6)+(0.03/3.0)=0.02

### Example 4

### Fludarabine/Compound K Combination Testing in A375 Melanoma Cells

Fludarabine, a purine analog, was tested in combination with Compound K in the melanoma cell line A375. Fludarabine was added 24 hours before Compound K in a 4 day assay. Results are shown hereafter; see FIG. 4 and FIG. 5. Synergy up to 65% is observed at concentrations tested. CI=0.03.

Fludarabine was added first, Compound K the next day (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.20 | 0.39 | 0.78 | 1.56 | 3.13 | 6.25 | 12.50 | 25.00 | 50.00 | 100.00 | 200 |
| 0 | 0.0 | 0.0 | −0.0 | 0.0 | 0.0 | 0.0 | 0.0 | 0.0 | −0.0 | 0.0 | −0.0 | 0.0 |
| 0.04 | 0.0 | 22.7 | 28.4 | 41.1 | 30.0 | 34.9 | 11.9 | 11.7 | 4.6 | 0.2 | −0.1 | −0.1 |
| 0.12 | 0.0 | 32.1 | 38.7 | 43.4 | 37.2 | 44.3 | 27.1 | 22.5 | 9.1 | 0.7 | −0.2 | −0.1 |
| 0.37 | 0.0 | 60.5 | 58.0 | 73.1 | 61.0 | 70.8 | 46.6 | 36.0 | 13.9 | 1.6 | −0.0 | −0.0 |
| 1.11 | 0.0 | 52.8 | 63.8 | 75.6 | 68.7 | 74.2 | 49.8 | 35.9 | 17.1 | 1.7 | −0.1 | 0.1 |
| 3.33 | 0.0 | 42.3 | 43.9 | 52.1 | 44.3 | 47.8 | 31.2 | 26.4 | 10.0 | 0.8 | −0.5 | −0.2 |
| 10.00 | 0.0 | 12.4 | 12.8 | 14.5 | 13.1 | 14.8 | 8.9 | 6.7 | 2.2 | −0.3 | −0.7 | −0.3 |
| 30 | 0.0 | 0.3 | 0.2 | 0.3 | 0.2 | 0.2 | −0.1 | −0.3 | −0.5 | −0.6 | −0.4 | −0.0 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | −0.8 | 30.7 | 41.4 | 24.9 | 24.9 | 45.4 | 43.0 | 28.8 | 4.7 | 0.1 | −0.0 |
| 0.12 | 0.0 | 29.8 | 53.3 | 68.3 | 47.4 | 45.2 | 71.1 | 64.4 | 41.6 | 6.2 | 0.2 | 0.1 |
| 0.37 | 0.0 | 37.3 | 57.7 | 74.5 | 56.0 | 57.0 | 73.8 | 65.2 | 42.3 | 6.0 | 0.1 | −0.1 |
| 1.11 | 0.0 | 41.5 | 64.2 | 73.2 | 57.6 | 57.1 | 81.3 | 68.5 | 44.6 | 6.4 | 0.1 | −0.0 |
| 3.33 | 0.0 | 26.1 | 39.5 | 50.9 | 37.9 | 41.1 | 50.8 | 48.5 | 30.4 | 4.6 | −0.1 | −0.1 |
| 10.00 | 0.0 | 5.1 | 10.9 | 11.9 | 9.1 | 8.6 | 12.4 | 11.9 | 7.2 | 0.7 | −0.4 | −0.2 |
| 30 | 0.0 | 0.1 | 0.3 | 0.3 | 0.1 | −0.1 | 0.2 | −0.0 | −0.0 | −0.3 | −0.2 | −0.0 |

Compound K: 1050=5.0 uM, Top=8874 RFU

Fludarabine: 1050=22.9 uM, Top=8227 RFU

Value of 50% effect=4276 RFU

50% Effect was achieved by combining 40 nM Compound K and 390 nM Fludarabine.

CI=[Compound K]/IC50_{compound} K+[Fludarabine]/IC50_{Fludarabine}=(0.04/5.0)+(0.39/22.9)=0.03

### Example 5

### Gemcitabine/Compound K Combination Testing in A375 Melanoma Cells

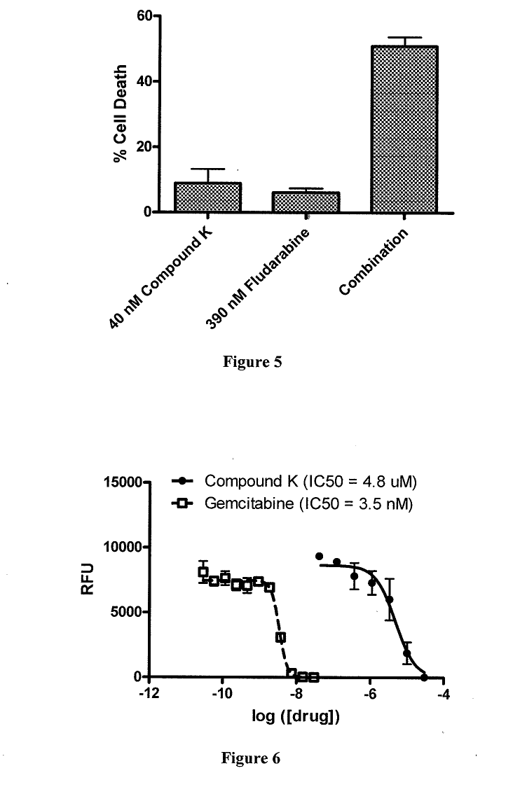

Gemcitabine, a pyrimidine, was tested in combination with Compound K in the melanoma cell line A375. Gemcitabine was added 24 hours before Compound K in a 4 day assay. Results are shown hereafter; see FIG. 6. Synergy up to 45% is observed at concentrations tested. CI=0.04.

Gemcitabine was added first, Compound K the next day (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00003 | 0.00006 | 0.00012 | 0.00023 | 0.00047 | 0.00094 | 0.00188 | 0.00375 | 0.0075 | 0.015 | 0.03 |
| 0 | 0.0 | 0.0 | 0.0 | 0.0 | 0.0 | 0.0 | 0.0 | 0.0 | −0.0 | 0.0 | 0.0 | 0.0 |
| 0.04 | 0.0 | 25.9 | 26.6 | 39.6 | 29.6 | 30.5 | 36.3 | 27.4 | 12.9 | 2.0 | 0.1 | 0.1 |
| 0.12 | 0.0 | 44.1 | 37.9 | 40.9 | 37.5 | 36.0 | 39.7 | 36.3 | 13.0 | 1.6 | −0.1 | 0.1 |
| 0.37 | 0.0 | 28.0 | 23.6 | 30.1 | 23.7 | 27.0 | 25.5 | 22.4 | 8.3 | 0.9 | −0.2 | 0.1 |
| 1.11 | 0.0 | 26.0 | 27.3 | 25.6 | 27.9 | 12.4 | 27.4 | 26.6 | 10.5 | 1.1 | −0.1 | 0.2 |
| 3.33 | 0.0 | 19.1 | 15.6 | 16.7 | 15.3 | 17.6 | 18.2 | 16.6 | 5.8 | 0.5 | −0.4 | −0.0 |
| 10.00 | 0.0 | 2.4 | 2.8 | 3.6 | 3.3 | 0.4 | 1.2 | 1.4 | 0.5 | −0.2 | −0.4 | −0.1 |
| 30 | 0.0 | −0.0 | −0.2 | −0.1 | −0.2 | −0.3 | −0.3 | −0.3 | −0.4 | −0.3 | −0.3 | −0.2 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | −13.5 | 14.8 | 6.8 | 2.4 | 1.0 | 12.1 | 2.4 | 4.4 | 0.8 | 0.1 | 0.0 |
| 0.12 | 0.0 | 39.2 | 44.4 | 46.3 | 42.1 | 42.5 | 46.8 | 44.0 | 20.8 | 2.3 | 0.1 | −0.1 |
| 0.37 | 0.0 | 47.9 | 52.5 | 51.7 | 47.5 | 51.2 | 58.8 | 48.4 | 26.5 | 2.6 | 0.0 | 0.0 |
| 1.11 | 0.0 | 46.4 | 50.7 | 51.3 | 47.8 | 48.8 | 54.1 | 48.5 | 25.2 | 2.4 | −0.0 | 0.0 |
| 3.33 | 0.0 | 43.2 | 49.7 | 48.5 | 47.5 | 46.0 | 52.8 | 47.8 | 24.8 | 2.3 | −0.2 | 0.0 |
| 10.00 | 0.0 | 14.8 | 15.9 | 17.5 | 16.8 | 17.2 | 19.1 | 17.3 | 8.8 | 0.6 | −0.3 | 0.0 |
| 30 | 0.0 | 0.3 | 0.2 | 0.2 | 0.2 | 0.1 | 0.0 | 0.0 | −0.3 | −0.4 | −0.2 | −0.2 |

Compound K: IC50=4.8 uM, Top=8646 RFU

Fludarabine: IC50=3.5 nM, Top=7461 RFU

Value of 50% effect=4027 RFU

50% Effect was achieved by combining 120 nM Compound K and 30 pM Gemcitabine

CI=[Compound K]/IC50_{Compound K}+[Gemcitabine]/IC50_{Gemcitabine}=(0.12/4.8)+(0.03/3.5)=0.04

### Example 6

### Paclitaxel/Compound K Combination Testing in A375 Melanoma Cells

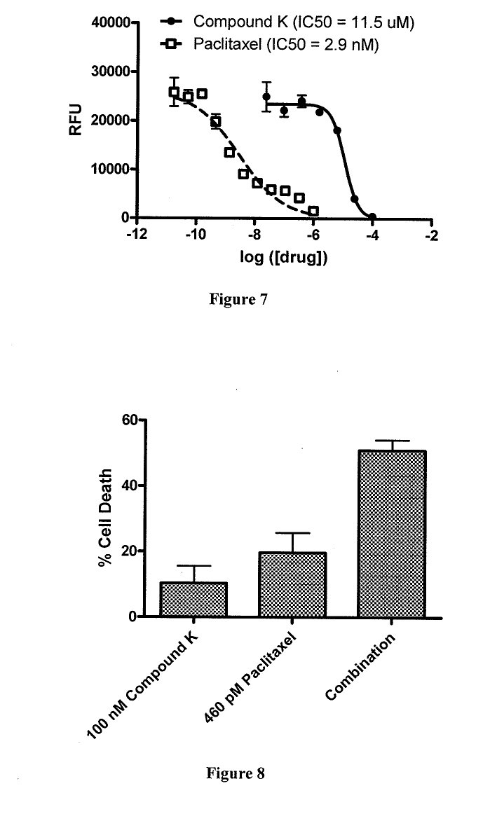

Paclitaxel, a mitotic inhibitor, was tested in combination with Compound K in the melanoma cell line A375. Paclitaxel was added 24 hours before Compound K in a 5 day assay. Results are shown hereafter; see FIG. 7 and FIG. 8. Synergy up to 30% is observed at concentrations tested. CI=0.17.

Paclitaxel was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00002 | 0.00005 | 0.00015 | 0.00046 | 0.0014 | 0.0041 | 0.012 | 0.037 | 0.11 | 0.33 | 1 |
| 0.00 | 0.0 | 0.0 | 0.0 | 0.0 | 0.0 | 0.0 | 0.0 | 0.0 | 0.0 | 0.0 | 0.0 | 0.0 |
| 0.02 | 0.0 | 18.0 | 31.4 | 36.1 | 29.8 | 14.6 | 11.3 | 10.2 | 1.9 | 4.0 | 3.7 | −2.9 |
| 0.10 | 0.0 | 20.1 | 8.7 | 14.5 | 16.5 | 21.4 | 11.5 | 8.1 | 1.2 | 1.1 | 0.2 | −6.0 |
| 0.39 | 0.0 | 0.0 | 0.0 | 14.9 | 31.3 | 26.5 | 17.1 | 13.6 | 5.9 | 4.6 | 5.6 | −3.2 |
| 1.56 | 0.0 | 10.3 | 19.7 | 27.2 | 26.8 | 22.3 | 14.0 | 12.0 | 5.9 | 6.4 | 5.2 | −1.5 |
| 6.25 | 0.0 | 26.4 | 27.5 | 29.6 | 20.6 | 17.8 | 11.4 | 10.6 | 7.0 | 8.2 | 6.9 | 0.5 |
| 25.00 | 0.0 | 0.7 | 4.8 | 5.8 | 3.6 | 2.7 | 1.2 | 1.3 | 0.6 | 1.0 | 1.3 | 0.6 |
| 100 | 0.0 | 0.5 | 1.0 | 1.0 | 0.8 | 0.6 | 0.4 | 0.6 | 0.2 | 0.2 | 0.1 | 0.1 |
| 0.00 | 0.0 | 0.0 | 0.0 | 0.0 | 0.0 | 0.0 | −0.0 | 0.0 | 0.0 | −0.0 | 0.0 | 0.0 |
| 0.02 | 0.0 | 13.8 | 29.4 | 28.2 | 39.7 | 17.2 | 8.2 | 5.6 | 3.8 | 3.0 | 2.7 | −0.8 |
| 0.10 | 0.0 | 16.8 | 20.1 | 19.9 | 21.3 | 4.0 | 1.1 | −0.7 | −0.4 | −1.5 | −4.5 | −7.5 |
| 0.39 | 0.0 | 16.6 | 4.9 | 15.8 | 19.0 | 9.3 | 4.6 | 2.1 | −0.7 | 0.5 | 0.4 | −4.6 |
| 1.56 | 0.0 | 22.7 | 14.8 | 19.1 | 17.4 | 4.7 | 3.7 | 1.4 | 1.3 | 2.1 | 1.5 | −2.6 |
| 6.25 | 0.0 | 18.0 | 15.8 | 18.2 | 16.4 | 5.0 | 2.7 | 3.0 | 4.2 | 5.9 | 4.4 | 0.4 |
| 25.00 | 0.0 | 1.1 | 0.8 | 1.2 | 1.5 | −0.8 | −1.1 | −0.7 | −0.9 | 0.1 | 0.7 | 0.4 |
| 100 | 0.0 | 0.3 | 0.4 | 0.6 | 0.5 | 0.2 | 0.2 | 0.1 | 0.3 | 0.1 | 0.2 | −0.2 |

Compound K: IC50=11.5 uM, Top=23452 RFU

Fludarabine: IC50=2.9 nM, Top=26000 RFU

Value of 50% effect=12363 RFU

50% Effect was achieved by combining 100 nM Compound K and 460 pM Paclitaxel.

CI=[Compound K]/IC50_{Compound K}+[Paelitaxel]/IC50_{Pachtaxel}=(0.1/11.5)+(0.46/2.9)=0.17

### Example 7

### Sunitinib/Compound K Combination Testing in A375 Melanoma Cells

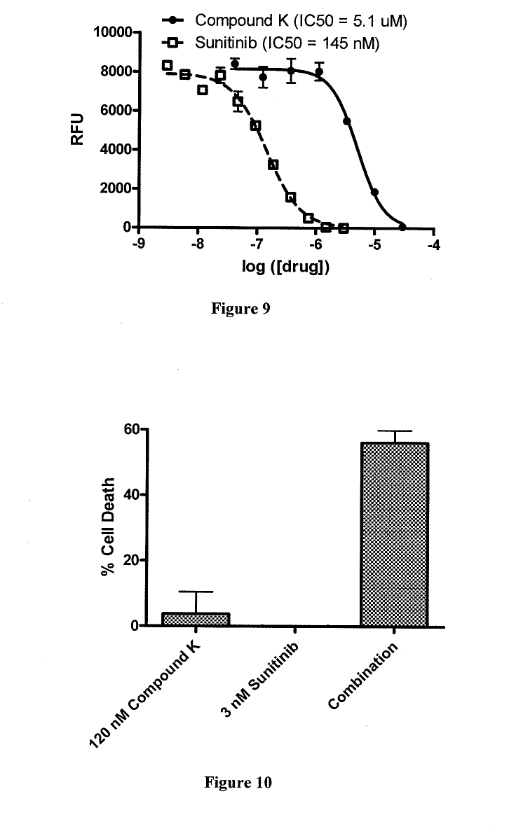

Sunitinib, a multi tyrosine-kinase inhibitor, was tested in combination with Compound K in the melanoma cell line A375. Sunitinib was added 24 hours before Compound K in a 4 day assay. Results are shown hereafter; see FIG. 9 and FIG. 10. Synergy up to 60% is observed at concentrations tested. CI=0.04.

Sunitinib was added first, Compound K the next day (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.003 | 0.006 | 0.012 | 0.023 | 0.047 | 0.094 | 0.188 | 0.375 | 0.75 | 1.5 | 3 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | 30.0 | 41.1 | 29.7 | 39.1 | 35.4 | 34.8 | 18.5 | 8.8 | 1.9 | −0.1 | 0.0 |
| 0.12 | 0.0 | 53.4 | 58.6 | 48.9 | 50.6 | 52.6 | 42.2 | 22.9 | 11.3 | 3.1 | −0.2 | −0.1 |
| 0.37 | 0.0 | 58.4 | 64.4 | 54.2 | 59.7 | 61.4 | 47.0 | 23.7 | 10.8 | 3.3 | −0.2 | −0.2 |
| 1.11 | 0.0 | 65.8 | 63.9 | 58.3 | 63.6 | 63.0 | 46.8 | 25.5 | 11.8 | 2.7 | −0.3 | −0.1 |
| 3.33 | 0.0 | 46.5 | 46.0 | 40.0 | 45.4 | 42.5 | 31.8 | 16.2 | 6.9 | 1.7 | −0.7 | −0.3 |
| 10.00 | 0.0 | 11.0 | 11.8 | 9.9 | 10.9 | 10.4 | 7.4 | 2.7 | 0.3 | −0.3 | −0.6 | −0.2 |
| 30 | 0.0 | 0.3 | 0.3 | 0.3 | 0.2 | 0.3 | 0.0 | −0.2 | −0.3 | −0.4 | −0.3 | −0.2 |
| Conc. | Conc. Drug μM | | | | | | | | | | | |
| K μM | 0 | 0.003 | 0.006 | 0.012 | 0.023 | 0.047 | 0.094 | 0.188 | 0.375 | 0.750 | 1.5 | 3 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | −5.0 | 21.8 | 30.4 | 44.8 | 32.7 | 39.2 | 27.9 | 13.2 | 3.7 | −0.0 | −0.1 |
| 0.12 | 0.0 | 34.5 | 42.2 | 40.0 | 54.0 | 33.7 | 37.1 | 22.8 | 10.2 | 2.9 | −0.2 | −0.1 |
| 0.37 | 0.0 | 48.7 | 55.1 | 51.4 | 61.7 | 42.6 | 41.9 | 25.6 | 11.7 | 4.0 | −0.2 | −0.1 |
| 1.11 | 0.0 | 50.3 | 58.5 | 50.5 | 65.0 | 45.7 | 42.8 | 26.4 | 11.6 | 3.5 | −0.1 | 0.1 |
| 3.33 | 0.0 | 37.5 | 38.2 | 34.8 | 45.0 | 31.3 | 28.6 | 16.1 | 6.7 | 1.4 | −0.5 | −0.1 |
| 10.00 | 0.0 | 14.9 | 15.8 | 14.3 | 17.2 | 11.6 | 10.0 | 5.0 | 1.7 | 0.3 | −0.7 | −0.3 |
| 30 | 0.0 | 0.1 | −0.1 | −0.2 | −0.2 | −0.2 | −0.1 | −0.3 | −0.4 | −0.5 | −0.4 | −0.1 |

Compound K: IC50=5.1 uM, Top=8150 RFU

Sunitinib: 1050=145 nM, Top=7914 RFU

Value of 50% effect=4016 RFU

50% Effect was achieved by combining 120 nM Compound K and 3 nM Sunitinib.

CI=[Compound K]/IC50_{Compound K}+[Sunitinib]/IC50_{Sunitimb}=(0.12/5.1)+(0.003/0.145)=0.04

### Example 8

### Vinblastine/Compound K Combination Testing in A375 Melanoma Cells

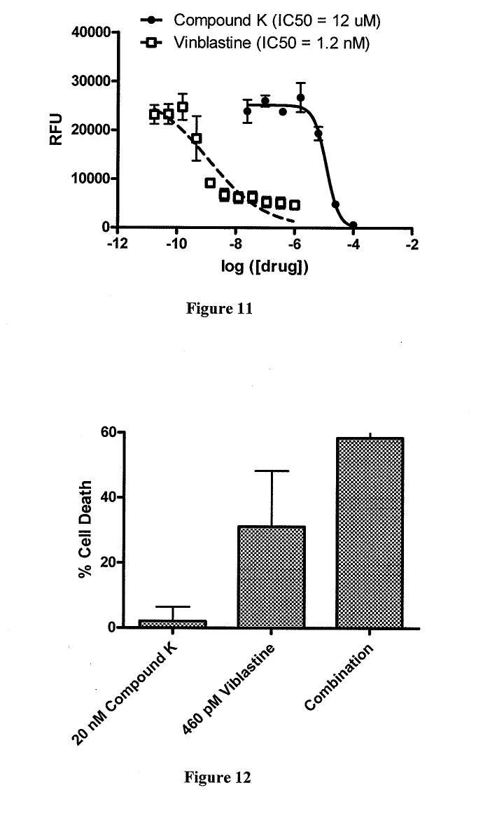

Vinblastine, a mitotic inhibitor, was tested in combination with Compound K in the melanoma cell line A375. Vinblastine was added 24 hours before Compound K in a 5 day assay. Results are shown hereafter; see FIG. 11 and FIG. 12. Synergy up to 35% is observed at concentrations tested. CI=0.39.

Vinblastine was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00002 | 0.00005 | 0.00015 | 0.00046 | 0.0014 | 0.0041 | 0.012 | 0.037 | 0.11 | 0.33 | 1 |
| 0.00 | 0.0 | 0.0 | 0.0 | 0.0 | 0.0 | 0.0 | 0.0 | −0.0 | 0.0 | 0.0 | 0.0 | 0.0 |
| 0.02 | 0.0 | 16.8 | 0.8 | 25.4 | 25.3 | 11.2 | 1.7 | 1.2 | 1.0 | 0.3 | −1.0 | 1.8 |
| 0.10 | 0.0 | −1.4 | −1.4 | 16.0 | 23.3 | 12.1 | 0.4 | −0.2 | −0.3 | −3.9 | −3.7 | −5.7 |
| 0.39 | 0.0 | −1.4 | −0.9 | 14.8 | 23.3 | 11.5 | 0.1 | 0.6 | 0.2 | −2.8 | −4.1 | −2.9 |
| 1.56 | 0.0 | 7.8 | 4.0 | 39.1 | 27.6 | 11.4 | −0.0 | −0.8 | 0.7 | 1.0 | −1.2 | 4.9 |
| 6.25 | 0.0 | 18.5 | 27.5 | 51.6 | 24.0 | 13.6 | 4.2 | 3.7 | 5.4 | 4.5 | 5.8 | 9.2 |
| 25.00 | 0.0 | 1.7 | 5.3 | 8.8 | 5.5 | 3.8 | 1.2 | 0.5 | 0.2 | −0.7 | −0.3 | 1.7 |
| 100 | 0.0 | 1.3 | 1.7 | 2.1 | 1.5 | 0.9 | 0.6 | 0.6 | 0.6 | 0.4 | 0.5 | 0.5 |
| 0.00 | 0.0 | 0.0 | 0.0 | 0.0 | 0.0 | 0.0 | 0.0 | 0.0 | 0.0 | 0.0 | 0.0 | 0.0 |
| 0.02 | 0.0 | 7.8 | 5.1 | 21.2 | 48.1 | 11.1 | 9.6 | 6.7 | 6.5 | 2.6 | 0.8 | −0.8 |
| 0.10 | 0.0 | −0.7 | 3.9 | 0.1 | 36.2 | 11.1 | 7.8 | 5.7 | 7.4 | 3.7 | 4.3 | −2.9 |
| 0.39 | 0.0 | 11.2 | −0.3 | 23.5 | 36.4 | 7.5 | 3.0 | 4.3 | 2.1 | 0.2 | −0.4 | −4.5 |
| 1.56 | 0.0 | −4.4 | 8.6 | 26.0 | 49.6 | 16.7 | 11.9 | 7.1 | 10.1 | 7.5 | 6.8 | 3.0 |
| 6.25 | 0.0 | 3.2 | 21.0 | 30.9 | 29.9 | 7.7 | 3.0 | 2.0 | 3.6 | 3.5 | 2.5 | 3.7 |
| 25.00 | 0.0 | −3.3 | 2.8 | 4.9 | 6.2 | 0.8 | −0.5 | −1.1 | −1.0 | −1.9 | −2.4 | −0.9 |
| 100 | 0.0 | 0.3 | 0.8 | 1.6 | 1.7 | 0.7 | 0.6 | 0.5 | 0.4 | 0.3 | 0.4 | 0.5 |

Compound K: 1050=12 uM, Top=25176 RFU

Vinblastine: 1050=1.2 nM, Top=28000 RFU

Value of 50% effect=13294 RFU

50% Effect was achieved by combining 20 nM Compound K and 460 pM Vinblastine.

CI=[Compound K]/IC50_{Compound K}+[Vinblastine]/IC50_{Vinblastine}=(0.02/12)+(0.46/1.2)=0.39

### Example 9

### 5-Fluorouracil/Compound K Combination Testing in MDA-MB-468 Breast Cancer Cells

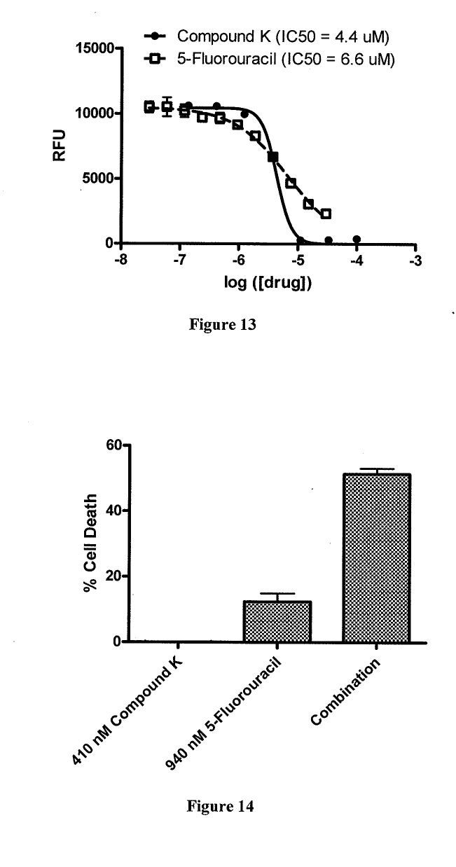

5-Fluorouracil, a pyrimidine analog, was tested in combination with Compound K in the breast cancer cell line MDA-MB-468. The effects of order of addition are examined. Results are shown hereafter; see FIG. 13 and FIG. 14. Synergy up to 40% is observed at concentrations tested. CI=0.18-0.24. Synergy did not depend on the order of addition.

5-Fluorouracil was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.03 | 0.06 | 0.12 | 0.23 | 0.47 | 0.94 | 1.88 | 3.75 | 7.50 | 15 | 30 |
| 0 | 0.0 | 0.0 | 0.0 | 0.0 | −0.0 | 0.0 | 0.0 | 0.0 | 0.0 | 0.0 | 0.0 | 0.0 |
| 0.14 | 0.0 | 5.4 | 16.5 | 28.1 | 21.4 | 29.7 | 28.8 | 18.4 | 21.7 | 13.1 | 9.5 | 2.4 |
| 0.41 | 0.0 | 25.1 | 34.7 | 43.0 | 36.1 | 39.2 | 39.2 | 30.7 | 27.2 | 18.9 | 17.1 | 1.7 |
| 1.2 | 0.0 | 28.6 | 38.3 | 44.6 | 36.9 | 39.3 | 36.2 | 31.7 | 29.3 | 20.4 | 17.3 | 2.8 |
| 3.7 | 0.0 | 20.1 | 24.3 | 26.8 | 24.9 | 25.4 | 26.7 | 22.9 | 19.7 | 16.0 | 11.0 | 2.5 |
| 11 | 0.0 | 1.4 | 1.6 | 1.9 | 1.0 | 0.6 | 0.2 | 0.9 | −0.8 | −1.3 | −1.4 | −3.9 |
| 33 | 0.0 | 2.3 | 0.7 | 2.9 | 1.2 | 2.0 | 1.6 | 0.9 | −0.8 | −0.4 | −1.3 | −3.0 |
| 100 | 0.0 | 2.6 | 2.3 | 2.4 | 2.5 | 1.4 | 0.6 | −0.9 | −0.5 | −1.8 | −1.8 | −3.1 |
| 0 | 0.0 | −0.0 | 0.0 | 0.0 | 0.0 | 0.0 | 0.0 | 0.0 | 0.0 | −0.0 | 0.0 | 0.0 |
| 0.14 | 0.0 | 12.2 | 14.5 | 17.8 | 15.7 | 25.7 | 24.1 | 25.5 | 28.2 | 15.5 | 8.2 | 0.9 |
| 0.41 | 0.0 | 21.4 | 32.9 | 34.2 | 36.0 | 37.0 | 35.0 | 36.2 | 26.8 | 18.8 | 16.8 | 1.2 |
| 1.2 | 0.0 | 16.2 | 29.8 | 33.1 | 32.7 | 35.9 | 32.5 | 31.3 | 28.2 | 18.7 | 14.3 | 1.2 |
| 3.7 | 0.0 | 10.7 | 18.6 | 23.9 | 26.5 | 25.8 | 23.8 | 24.3 | 20.1 | 16.1 | 10.2 | 3.4 |
| 11 | 0.0 | 0.7 | 1.7 | 2.0 | 1.2 | 0.9 | 0.4 | 1.1 | −0.2 | −0.3 | −0.6 | −3.9 |
| 33 | 0.0 | 1.5 | 0.5 | 2.6 | 1.4 | 2.4 | 2.2 | 1.3 | −0.3 | 0.1 | −0.4 | −3.6 |
| 100 | 0.0 | 2.1 | 1.8 | 1.9 | 2.2 | 0.9 | 0.4 | −0.6 | −0.7 | −2.3 | −1.7 | −3.3 |

Compound K: 1050=4.4 uM, Top=10446 RFU

5-Fluorouracil: 1050=6.6 uM, Top=10485 RFU

Value of 50% effect=5233 RFU

50% Effect was achieved by combining 410 nM Compound K and 940 nM 5-Fluorouracil.

CI=[Compound K]/IC50_{Compound K}α[5-Fluorouracil]/IC50_{5-Fluorouracil}=(0.4/14.4)+(0.94/6.6)=0.24

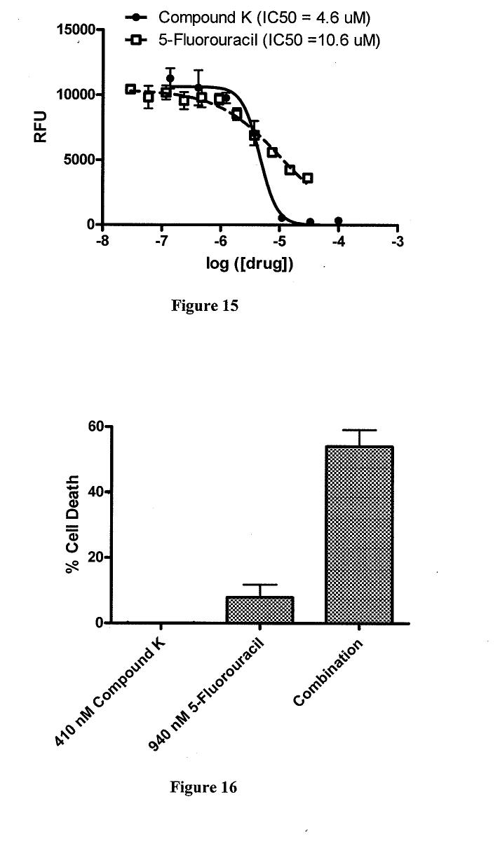

Compound K was added first, 5-Fluorouracil the next day (5 day assay in total). Results indicate the degree of inhibitory effect found with agent combination, where a positive value denotes synergy and a negative value antagonism. See FIG. 15 and FIG. 16. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.03 | 0.06 | 0.12 | 0.23 | 0.47 | 0.94 | 1.88 | 3.75 | 7.50 | 15 | 30 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 10.3 | −2.5 | 0.4 | 0.1 | 9.9 | 15.5 | 13.4 | 13.5 | 13.5 | −1.7 | 3.3 |
| 0.41 | 0.0 | 8.3 | 20.0 | 27.6 | 22.7 | 31.4 | 38.1 | 35.0 | 25.1 | 18.4 | 14.4 | 3.3 |
| 1.2 | 0.0 | 15.8 | 21.6 | 28.1 | 24.6 | 27.2 | 27.4 | 28.3 | 18.7 | 15.3 | 9.1 | −1.7 |
| 3.7 | 0.0 | 20.7 | 24.3 | 28.5 | 26.5 | 26.6 | 27.2 | 24.5 | 18.5 | 12.3 | 13.1 | 8.1 |
| 11 | 0.0 | 2.2 | 3.4 | 3.3 | 2.5 | 2.3 | 1.8 | 2.3 | 0.6 | 0.2 | −0.8 | −0.8 |
| 33 | 0.0 | 1.2 | −0.3 | 2.1 | 0.1 | 1.2 | 0.2 | −0.0 | −1.6 | −0.9 | −1.7 | −2.0 |
| 100 | 0.0 | 1.1 | 1.2 | 1.4 | 1.6 | 0.7 | −0.1 | −0.7 | −0.5 | −1.8 | −1.7 | −2.2 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 7.1 | 0.4 | 17.4 | 16.5 | 10.1 | 19.0 | 24.6 | 22.4 | 24.4 | 2.3 | 4.2 |
| 0.41 | 0.0 | 12.4 | 15.9 | 25.8 | 18.1 | 30.0 | 36.6 | 30.7 | 29.2 | 19.6 | 22.2 | 1.6 |
| 1.2 | −0.0 | 15.8 | 19.0 | 31.3 | 29.1 | 29.3 | 37.8 | 35.5 | 27.3 | 21.8 | 20.2 | 2.9 |
| 3.7 | 0.0 | 8.0 | 11.8 | 15.5 | 16.8 | 20.6 | 23.6 | 23.0 | 16.8 | 19.2 | 13.2 | 0.1 |
| 11 | 0.0 | 0.7 | 1.1 | 2.1 | 1.2 | 0.2 | 0.2 | 0.9 | −0.7 | −0.2 | −1.7 | −2.4 |
| 33 | 0.0 | 1.3 | −0.4 | 1.8 | 0.2 | 1.3 | 0.4 | −0.2 | −1.6 | −0.3 | −1.5 | −2.5 |
| 100 | 0.0 | 1.5 | 0.7 | 1.2 | 1.4 | 0.1 | −0.0 | −0.5 | −0.2 | −1.5 | −1.7 | −2.8 |

Compound K: 1050=4.6 uM, Top=10630 RFU

5-Fluorouracil: 1050=10.6 uM, Top=10384 RFU

Value of 50% effect=5254 RFU

50% Effect was achieved by combining 410 nM Compound K and 940 nM 5-Fluorouracil.

CI=[Compound K]/IC50_{Compound K}+[5-Fluorouracil]/IC50_{5-Fluorouracil}=(0.41/4.6)+(0.94/10.6)=0.18

### Example 10

### Cisplatin/Compound K Combination Testing in MDA-MB-468 Breast Cancer Cells

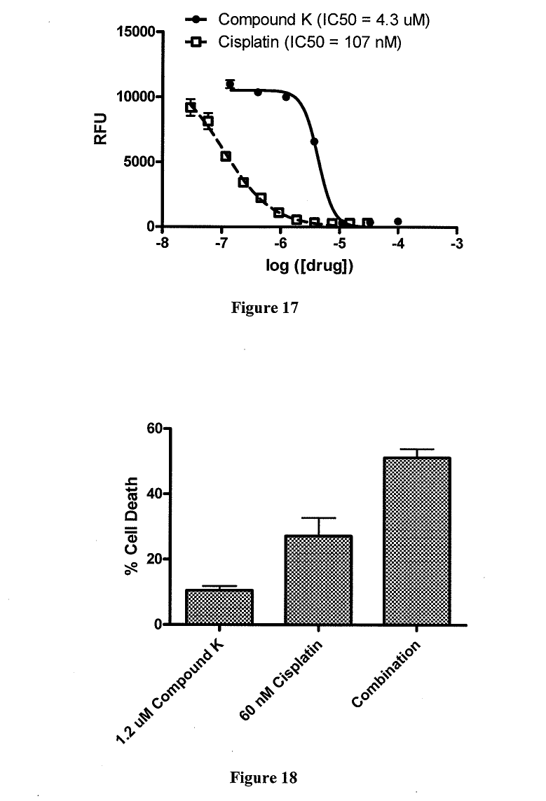

Cisplatin, an alkylating-like agent, was tested in combination with Compound K in the breast cancer cell line MDA-MB-468. The effects of order of addition are examined. Results are shown hereafter; see FIG. 17 and FIG. 18. Synergy up to 15% is observed at concentrations tested. CI=0.3-0.84. Synergy did not depend on the order of addition.

Cisplatin was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.03 | 0.06 | 0.12 | 0.23 | 0.47 | 0.94 | 1.88 | 3.75 | 7.50 | 15 | 30 |
| 0 | 0.0 | 0.0 | 0.0 | −0.0 | 0.0 | 0.0 | 0.0 | 0.0 | 0.0 | 0.0 | 0.0 | 0.0 |
| 0.14 | 0.0 | 1.4 | 12.5 | 5.9 | 4.6 | 7.2 | 2.9 | 1.2 | 0.4 | 0.5 | 1.2 | 0.7 |
| 0.41 | 0.0 | 5.0 | 9.6 | 12.5 | 9.6 | 8.5 | 4.3 | 2.3 | 0.8 | 0.4 | 0.9 | 0.9 |
| 1.2 | 0.0 | 11.9 | 10.5 | 13.2 | 12.7 | 8.6 | 3.8 | 1.9 | 0.4 | 1.2 | 0.9 | 0.7 |
| 3.7 | 0.0 | 9.3 | 14.0 | 8.8 | 5.2 | 4.3 | 2.3 | 1.4 | 0.7 | 0.2 | 0.4 | 0.7 |
| 11 | 0.0 | −0.2 | 0.3 | 0.3 | −0.3 | −0.4 | −0.8 | −0.2 | −0.6 | −0.6 | −0.3 | −0.5 |
| 33 | 0.0 | 1.2 | −0.2 | 1.2 | −0.4 | 0.2 | −0.4 | −0.3 | −1.6 | −0.7 | −0.5 | −0.9 |
| 100 | 0.0 | 0.8 | 0.7 | 0.4 | −0.2 | −1.5 | −1.4 | −1.7 | −1.6 | −2.5 | −1.3 | −1.8 |
| 0 | 0.0 | 0.0 | 0.0 | 0.0 | −0.0 | 0.0 | 0.0 | 0.0 | 0.0 | −0.0 | 0.0 | 0.0 |
| 0.14 | 0.0 | −1.6 | 0.9 | 7.0 | 4.4 | 7.6 | 2.0 | 1.4 | 0.8 | 0.4 | 1.7 | 0.9 |
| 0.41 | 0.0 | 3.6 | 10.1 | 16.4 | 11.4 | 10.4 | 4.7 | 2.6 | 0.5 | 0.4 | 1.1 | 1.3 |
| 1.2 | 0.0 | 7.7 | 16.6 | 23.2 | 18.9 | 11.9 | 5.1 | 2.4 | 1.1 | 1.6 | 1.2 | 1.1 |
| 3.7 | 0.0 | 10.7 | 19.8 | 19.5 | 10.3 | 7.3 | 3.2 | 2.0 | 0.7 | 0.4 | −0.6 | 0.2 |
| 11 | 0.0 | −0.3 | 0.4 | 0.3 | −0.5 | −0.4 | −2.0 | 0.2 | −1.9 | −2.3 | −2.0 | −2.2 |
| 33 | 0.0 | 1.3 | −0.4 | 0.9 | −0.7 | −0.5 | −1.0 | −1.6 | −2.2 | −2.3 | −2.4 | −2.3 |
| 100 | 0.0 | 0.7 | 0.6 | −0.2 | −0.6 | −2.2 | −2.7 | −4.1 | −3.5 | −4.3 | −3.2 | −3.6 |

Compound K: IC50=4.3 uM, Top=10513 RFU

5-Fluorouracil: IC50=107 nM, Top=11803 RFU

Value of 50% effect=5579 RFU

50% Effect was achieved by combining 1.2 uM Compound K and 60 nM Cisplatin.

CI=[Compound K]/IC50_{Compound K}+[Cisplatin]/IC50_{Cisplatin}=(1.2/4.3)+(0.06/0.107)=0.84

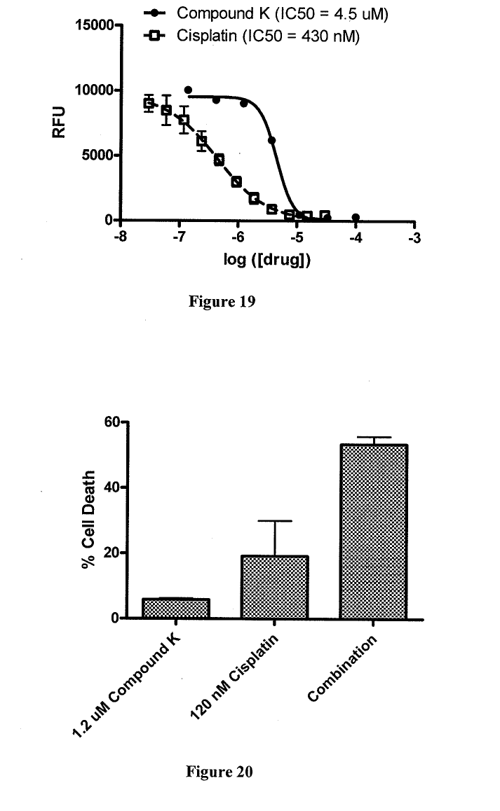

Compound K was added first, Cisplatin the next day (5 day assay in total). Results indicate the degree of inhibitory effect found with agent combination, where a positive value denotes synergy and a negative value antagonism; see FIG. 19 and FIG. 20. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.03 | 0.06 | 0.12 | 0.23 | 0.47 | 0.94 | 1.88 | 3.75 | 7.50 | 15 | 30 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 28.5 | 32.5 | 35.0 | 21.1 | 21.0 | 19.8 | 13.3 | 4.7 | 2.5 | 2.2 | 1.5 |
| 0.41 | 0.0 | 25.7 | 30.2 | 35.5 | 19.3 | 19.7 | 20.2 | 13.4 | 5.0 | 3.2 | 1.5 | 1.7 |
| 1.2 | 0.0 | 21.0 | 24.0 | 38.5 | 25.7 | 21.8 | 20.4 | 14.2 | 5.7 | 3.7 | 1.6 | 1.7 |
| 3.7 | −0.0 | 13.9 | 22.6 | 27.0 | 21.3 | 21.0 | 13.2 | 9.6 | 3.8 | 2.2 | 1.0 | 0.7 |
| 11 | 0.0 | 1.7 | 2.1 | 2.6 | 1.2 | 0.4 | −1.0 | 0.0 | −1.5 | −0.7 | −2.0 | −2.0 |
| 33 | 0.0 | 1.7 | −0.1 | 2.0 | 0.1 | 0.7 | −0.8 | −1.1 | −2.6 | −1.4 | −2.2 | −2.9 |
| 100 | 0.0 | 1.8 | 1.4 | 1.4 | 1.4 | −0.3 | −1.5 | −2.3 | −1.9 | −3.1 | −2.7 | −3.5 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | −6.0 | 4.0 | 3.1 | 6.0 | 5.8 | 7.1 | 3.1 | 1.4 | 0.8 | 1.0 | 0.6 |
| 0.41 | 0.0 | −2.7 | −3.4 | 1.9 | 4.1 | 5.8 | 7.3 | 3.4 | 0.9 | 0.8 | 0.6 | 0.6 |
| 1.2 | 0.0 | −1.6 | 3.9 | 5.9 | 6.0 | 6.2 | 8.0 | 3.2 | 1.5 | 1.4 | 0.5 | 0.8 |
| 3.7 | 0.0 | 5.5 | 2.3 | 1.5 | 6.0 | 9.3 | 5.5 | 2.6 | 1.2 | 0.6 | −0.1 | −0.0 |
| 11 | 0.0 | 0.2 | 0.7 | 0.9 | 0.4 | −0.5 | −1.2 | −0.4 | −1.6 | −1.2 | −2.2 | −1.9 |
| 33 | 0.0 | 1.0 | −0.8 | 1.5 | −0.8 | 0.1 | −0.9 | −1.1 | −2.6 | −1.6 | −2.5 | −2.8 |
| 100 | 0.0 | 0.7 | 0.2 | 0.2 | 0.2 | −1.2 | −2.0 | −2.9 | −2.6 | −3.7 | −3.4 | −3.8 |

Compound K: IC50=4.5 uM, Top=9530 RFU

Cisplatin: IC50=430 nM, Top=9646 RFU

Value of 50% effect=4794 RFU

50% Effect was achieved by combining 1.2 uM Compound K and 120 nM Cisplatin.

CI=[Compound K]/IC50_{Compound K}+[Cisplatin]/IC50_{Cisplatin}=(1.2/4.5)+(0.12/0.43)=0.3

### Example 11

### Doxorubicin/Compound K Combination Testing in MDA-MB-468 Breast Cancer cells

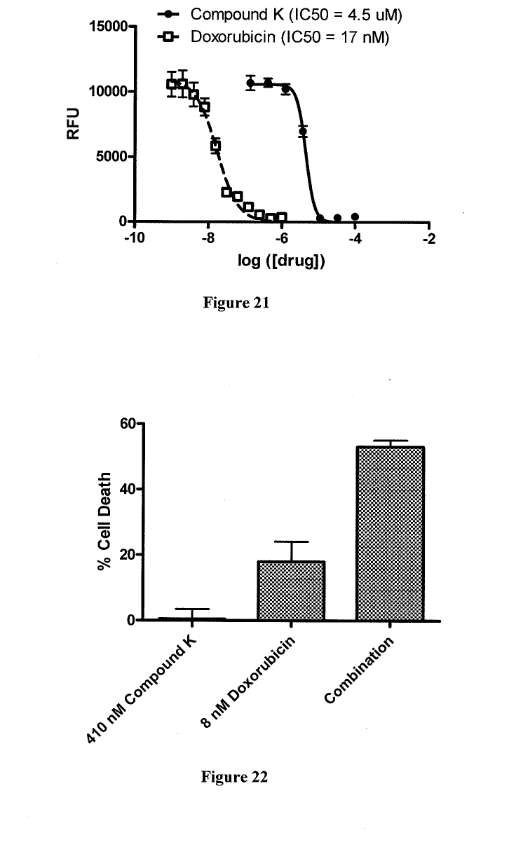

Doxorubicin, an anthracycline, was tested in combination with Compound K in the breast cancer cell line MDA-MB-468. The effects of order of addition are examined. Results are shown hereafter; see FIG. 21 and FIG. 22. Synergy up to 30% is observed. CI=0.56-0.76. Synergy did not depend on the order of addition.

Doxorubicin was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.001 | 0.002 | 0.004 | 0.008 | 0.016 | 0.031 | 0.063 | 0.125 | 0.25 | 0.5 | 1 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 14.2 | 11.7 | 10.1 | −2.4 | 7.3 | 11.5 | 8.6 | 3.7 | 1.1 | 1.3 | 1.3 |
| 0.41 | 0.0 | 10.9 | 30.4 | 31.3 | 29.1 | 20.0 | 15.1 | 11.4 | 5.5 | 1.8 | 1.1 | 1.7 |
| 1.2 | −0.0 | 20.0 | 36.3 | 24.8 | 28.9 | 25.2 | 14.3 | 11.6 | 6.6 | 3.8 | 1.4 | 1.7 |
| 3.7 | 0.0 | 17.8 | 27.6 | 24.4 | 22.2 | 24.4 | 9.0 | 7.4 | 5.4 | 2.5 | 0.3 | 0.5 |
| 11 | 0.0 | 1.3 | 1.8 | 2.4 | 1.8 | 1.3 | −0.4 | 0.5 | −0.9 | −0.9 | −1.2 | −1.7 |
| 33 | 0.0 | 2.0 | 1.1 | 2.6 | 1.4 | 1.2 | −0.3 | −0.7 | −2.2 | −1.6 | −2.6 | −2.1 |
| 100 | 0.0 | 1.3 | 1.3 | 1.6 | 1.3 | 0.1 | −2.1 | −3.0 | −2.5 | −3.0 | −2.9 | −2.8 |
| 0 | 0.0 | −0.0 | 0.0 | 0.0 | 0.0 | −0.0 | 0.0 | 0.0 | 0.0 | 0.0 | 0.0 | 0.0 |
| 0.14 | 0.0 | −1.8 | 11.2 | 10.6 | 8.3 | 2.8 | 7.0 | 7.8 | 3.6 | 1.1 | 1.3 | 0.6 |
| 0.41 | 0.0 | −11.0 | 22.3 | 18.3 | 27.6 | 19.8 | 11.5 | 11.0 | 5.0 | 1.6 | 1.2 | 1.3 |
| 1.2 | 0.0 | 0.0 | 18.5 | 25.1 | 20.5 | 20.3 | 10.4 | 10.8 | 5.7 | 3.1 | 1.1 | 1.0 |
| 3.7 | 0.0 | −3.8 | 20.1 | 16.6 | 21.4 | 19.2 | 5.7 | 6.6 | 3.9 | 1.3 | −0.1 | 0.1 |
| 11 | 0.0 | 1.0 | 1.5 | 1.9 | 0.8 | 0.1 | −1.1 | −0.5 | −1.4 | −1.4 | −1.2 | −2.2 |
| 33 | −0.0 | 1.4 | 0.5 | 2.2 | 0.7 | 0.3 | −0.2 | −1.0 | −2.6 | −1.9 | −2.2 | −2.5 |
| 100 | 0.0 | 0.9 | 1.1 | 0.9 | 0.6 | −1.3 | −2.5 | −3.7 | −3.1 | −4.5 | −4.2 | −3.8 |

Compound K: 1050=4.5 uM, Top=10577 RFU

Doxorubicin: IC50=17 nM, Top=10942 RFU

Value of 50% effect=5380 RFU

50% Effect was achieved by combining 410 nM Compound K and 8 nM Doxorubicin.

CI=[Compound K]/IC⁵⁰_{Compound K}+[Doxorubicin]/IC50_{Doxorubicin}=(0.41/4.5)+(0.008/0.017)=0.56

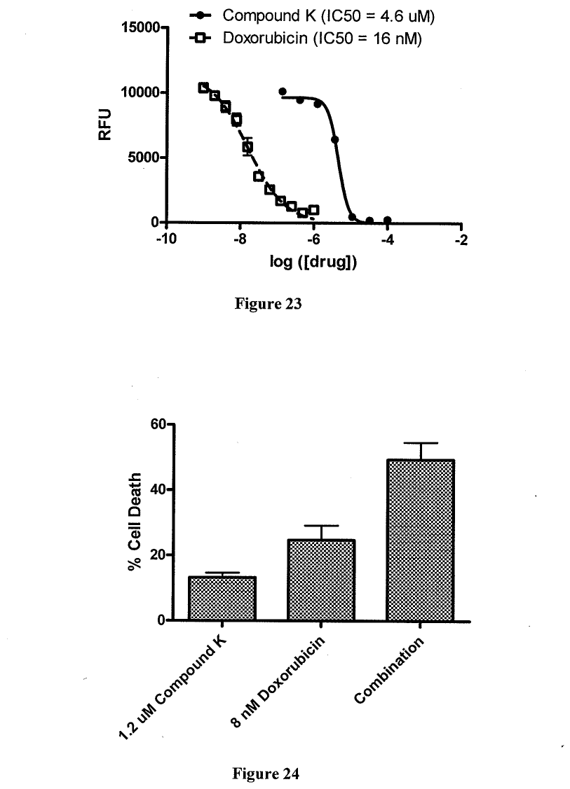

Compound K was added first, Doxorubicin the next day (5 day assay in total). Results indicate the degree of inhibitory effect found with agent combination, where a positive value denotes synergy and a negative value antagonism. See FIG. 23 and FIG. 24. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.001 | 0.002 | 0.004 | 0.008 | 0.016 | 0.031 | 0.063 | 0.125 | 0.25 | 0.5 | 1 |
| 0 | 0.0 | 0.0 | −0.0 | −0.0 | 0.0 | 0.0 | 0.0 | 0.0 | 0.0 | 0.0 | 0.0 | 0.0 |
| 0.14 | 0.0 | 8.9 | 10.4 | 15.7 | 15.7 | 17.5 | 10.6 | 11.1 | 8.0 | 3.4 | 2.7 | 1.3 |
| 0.41 | 0.0 | 25.3 | 10.4 | 20.6 | 16.6 | 19.4 | 11.1 | 11.0 | 5.9 | 4.5 | 2.4 | 2.0 |
| 1.2 | 0.0 | 10.6 | 18.5 | 13.8 | 12.9 | 21.0 | 11.3 | 12.7 | 9.0 | 5.6 | 2.8 | 2.6 |
| 3.7 | 0.0 | 10.8 | 9.7 | 7.4 | 12.5 | 13.2 | 9.8 | 7.3 | 5.3 | 3.5 | 1.7 | 1.9 |
| 11 | 0.0 | 2.0 | 1.9 | 2.6 | 1.8 | 0.2 | −1.3 | −0.6 | −1.6 | −1.3 | −2.1 | −2.2 |
| 33 | 0.0 | 1.5 | −0.0 | 1.7 | 0.0 | 0.7 | −0.9 | −1.5 | −2.8 | −1.8 | −2.4 | −3.3 |
| 30 | 0.0 | 1.7 | 0.9 | 1.0 | 1.3 | −0.6 | −1.4 | −2.1 | −1.7 | −2.8 | −2.5 | −3.5 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 11.9 | 17.2 | 5.2 | 6.9 | 3.9 | 1.9 | 9.0 | 3.0 | 3.3 | 2.8 | 1.2 |
| 0.41 | 0.0 | 12.0 | 26.3 | 17.1 | 13.3 | 8.9 | 8.7 | 10.6 | 3.6 | 4.2 | 2.9 | 1.2 |
| 1.2 | 0.0 | 23.0 | 27.8 | 18.5 | 15.4 | 9.8 | 12.7 | 10.5 | 5.1 | 6.1 | 3.2 | 2.3 |
| 3.7 | 0.0 | 12.3 | 17.8 | 10.5 | 8.5 | 7.9 | 7.5 | 7.2 | 3.7 | 4.6 | 2.5 | 2.1 |
| 11 | 0.0 | 1.1 | 1.7 | 1.5 | 0.6 | −0.6 | −2.4 | −0.8 | −1.7 | −0.7 | −1.8 | −1.6 |
| 33 | −0.0 | 1.6 | −0.1 | 1.6 | −0.3 | 0.3 | −1.1 | −1.2 | −2.5 | −1.4 | −2.0 | −2.6 |
| 100 | 0.0 | 1.7 | 1.2 | 1.1 | 1.3 | −0.7 | −1.6 | −2.1 | −2.0 | −3.1 | −2.7 | −3.5 |

Compound K: IC50=4.6 uM, Top=9652 RFU

Doxorubicin: IC50=16 nM, Top=11475 RFU

Value of 50% effect=5282 RFU

50% Effect was achieved by combining 1.2 uM Compound K and 8 nM Doxorubicin.

CI=[Compound K]/IC50_{Compound K}+[Doxorubicin]/IC50_{Doxorubicin}=(1.2/4.6)+(0.008/0.016)=0.76

### Example 12

### Gemcitabine/Compound K Combination Testing in MDA-MB-468 Breast Cancer Cells

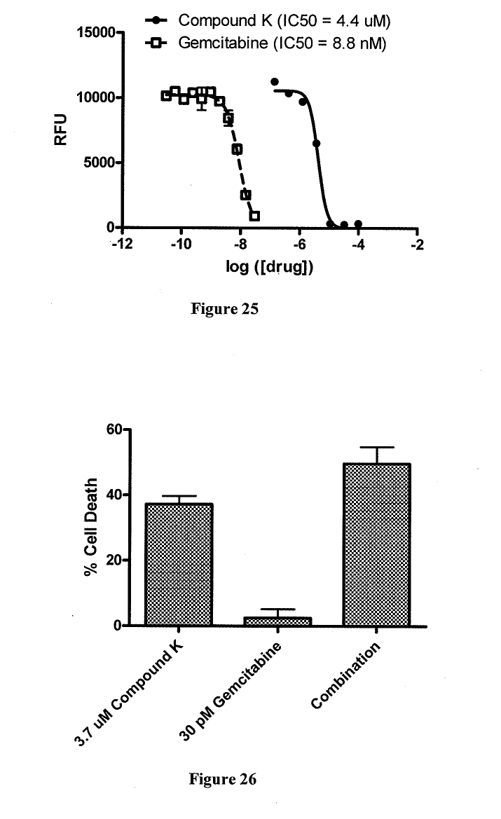

Gemcitabine, a pyrimidine analog, was tested in combination with Compound K in the breast cancer cell line MDA-MB-468. The effects of order of addition are examined. Results are shown hereafter; see FIG. 25 and FIG. 26. Synergy up to 30% is observed at concentrations tested. CI=0.29-0.84. Synergy did not depend on the order of addition.

Gemcitabine was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00003 | 0.00006 | 0.00012 | 0.00023 | 0.00047 | 0.00094 | 0.0019 | 0.0038 | 0.0075 | 0.015 | 0.03 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | −4.7 | 13.2 | 6.3 | 16.6 | 18.5 | 13.2 | 9.0 | 12.3 | 8.2 | 3.9 | 0.1 |
| 0.41 | 0.0 | 6.1 | 19.6 | 9.8 | 16.1 | 23.2 | 25.8 | 17.1 | 10.8 | −5.5 | 8.5 | 1.7 |
| 1.2 | 0.0 | −2.0 | 21.3 | 20.6 | 19.7 | 34.2 | 25.4 | 14.8 | 15.3 | 10.2 | 10.3 | 2.7 |
| 3.7 | 0.0 | −0.2 | 13.8 | 14.8 | 16.9 | 23.4 | 19.1 | 11.8 | 11.5 | 17.0 | 8.2 | 3.2 |
| 11 | 0.0 | 1.4 | 1.1 | 2.0 | 1.7 | 1.9 | 0.8 | 1.0 | 1.1 | 0.9 | −0.2 | −1.7 |
| 33 | 0.0 | 1.2 | 0.5 | 2.0 | 1.4 | 1.3 | −0.1 | 0.3 | −0.7 | −0.5 | −1.5 | −2.3 |
| 100 | 0.0 | 0.5 | 0.8 | 0.7 | 1.5 | 0.4 | −0.1 | 0.2 | −0.7 | −1.5 | −2.3 | −2.9 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 5.6 | 24.7 | 21.2 | 26.3 | 20.2 | 31.1 | 30.2 | 27.6 | 19.9 | 11.2 | 0.8 |
| 0.41 | 0.0 | 14.9 | 23.4 | 24.7 | 32.2 | 16.3 | 23.0 | 19.3 | 20.2 | 20.3 | 12.4 | 0.8 |
| 1.2 | 0.0 | 13.3 | 19.5 | 20.9 | 29.9 | 23.1 | 26.2 | 19.6 | 19.4 | 23.7 | 12.4 | 1.5 |
| 3.7 | 0.0 | 11.8 | 11.5 | 16.3 | 24.4 | 19.9 | 25.4 | 22.0 | 20.6 | 24.8 | 11.5 | 3.1 |
| 11 | 0.0 | 1.3 | 0.8 | 1.8 | 1.2 | 1.7 | 0.9 | 1.7 | 2.2 | 1.6 | 0.4 | −1.4 |
| 33 | 0.0 | 1.1 | 0.8 | 2.2 | 1.9 | 1.6 | 0.6 | 1.1 | 0.3 | 0.5 | −1.2 | −2.2 |
| 100 | 0.0 | 0.3 | 0.6 | 0.3 | 1.6 | 1.1 | 0.9 | 1.0 | 0.0 | −1.5 | −2.6 | −3.3 |

Compound K: 1050=4.4 uM, Top=10572 RFU

Gemcitabine: IC50=8.8 nM, Top=10229 RFU

Value of 50% effect=5200 RFU

50% Effect was achieved by combining 3.7 uM Compound K and 30 pM Gemcitabine.

CI=[Compound K]/IC50_{Compound K}+[Gemcitabine]/IC50_{Gemcitabine}=(3.7/4.4)+(0.03/8.8)=0.84

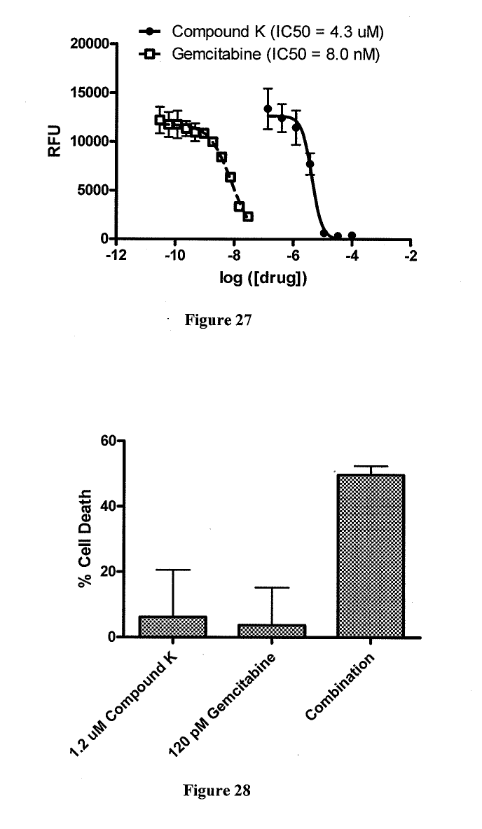

Compound K was added first, Gemcitabine the next day (5 day assay in total). Results indicate the degree of inhibitory effect found with agent combination, where a positive value denotes synergy and a negative value antagonism. See FIG. 27 and FIG. 28. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00003 | 0.00006 | 0.00012 | 0.00023 | 0.00047 | 0.00094 | 0.0019 | 0.0038 | 0.0075 | 0.015 | 0.03 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 2.3 | 8.5 | 17.3 | 16.3 | 11.8 | 10.3 | 10.9 | 13.3 | 8.5 | 1.1 | 5.2 |
| 0.41 | 0.0 | 0.9 | 17.1 | 26.1 | 20.7 | 20.0 | 18.9 | 17.0 | 15.8 | 12.3 | 1.9 | 4.6 |
| 1.2 | 0.0 | 8.6 | 21.1 | 27.4 | 20.5 | 21.4 | 21.1 | 20.7 | 16.7 | 12.5 | 8.2 | 5.4 |
| 3.7 | 0.0 | 2.0 | 11.1 | 15.3 | 10.7 | 11.1 | 13.3 | 13.9 | 13.0 | 10.1 | 6.9 | 4.7 |
| 11 | 0.0 | −0.4 | 1.2 | 1.7 | 1.2 | 0.6 | 0.3 | 0.9 | 0.9 | 0.5 | −1.1 | −1.6 |
| 33 | 0.0 | 0.9 | −0.2 | 1.9 | 0.3 | 1.2 | 0.5 | 0.8 | −0.7 | −0.2 | −1.2 | −1.7 |
| 100 | 0.0 | 0.8 | 0.8 | 1.0 | 1.2 | 0.5 | 0.1 | −0.5 | −0.2 | −1.4 | −1.4 | −2.1 |
| 0 | 0.0 | 0.0 | 0.0 | 0.0 | 0.0 | 0.0 | 0.0 | 0.0 | 0.0 | −0.0 | 0.0 | 0.0 |
| 0.14 | 0.0 | 20.3 | 18.2 | 17.1 | 17.8 | 17.9 | 14.8 | 18.4 | 7.0 | −4.4 | −8.0 | 6.7 |
| 0.41 | 0.0 | 23.2 | 33.8 | 32.9 | 34.0 | 28.5 | 32.3 | 32.1 | 22.1 | 17.3 | −7.7 | 6.6 |
| 1.2 | 0.0 | 11.5 | 21.8 | 27.3 | 25.4 | 17.1 | 27.8 | 27.2 | 18.3 | 18.1 | −5.2 | 5.7 |
| 3.7 | 0.0 | 5.1 | 21.0 | 23.3 | 20.7 | 13.9 | 17.8 | 16.3 | 13.7 | 10.8 | 4.5 | 5.5 |
| 11 | 0.0 | 1.4 | 2.3 | 2.4 | 2.1 | 1.2 | 0.8 | 1.6 | 0.3 | −0.0 | −2.7 | −2.4 |
| 33 | 0.0 | 1.5 | 0.1 | 2.5 | 0.6 | 1.3 | 0.6 | 0.4 | −1.3 | −0.7 | −1.9 | −2.6 |
| 100 | 0.0 | 1.5 | 1.2 | 1.6 | 2.0 | 0.9 | 0.3 | −0.4 | −0.4 | −1.8 | −2.4 | −3.0 |

Compound K: 1050=4.3 uM, Top=12460 RFU

Gemcitabine: 1050=8 nM, Top=11772 RFU

Value of 50% effect=6103 RFU

50% Effect was achieved by combining 1.2 uM Compound K and 120 pM Gemcitabine.

CI=[Compound K]/IC50_{Compound K}+[Gemcitabine]/IC50_{Gemcitabine}=(1.2/4.3)+(0.12/8)=0.29

### Example 13

### Vinblastine/Compound K Combination Testing in MIA PaCa-2 Pancreatic Cancer Cells

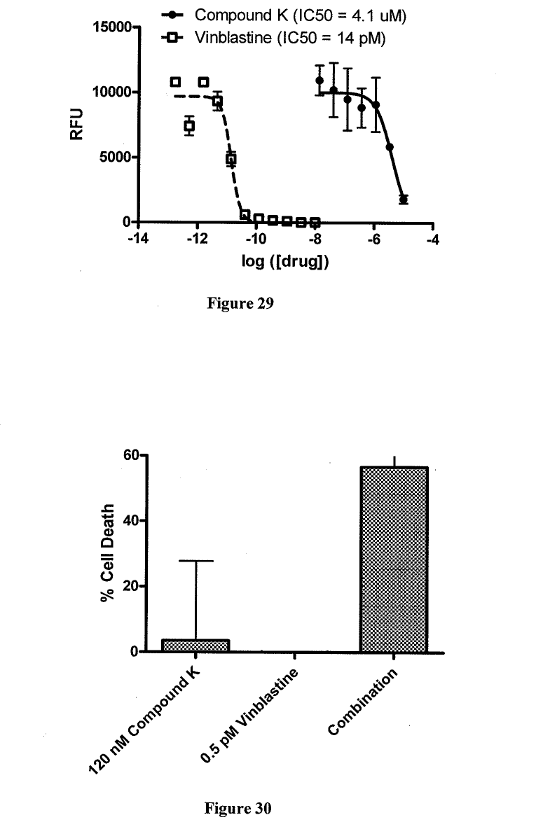

Vinblastine, a mitotic inhibitor, was tested in combination with Compound K in the pancreatic cancer cell line MIA PaCa-2. Vinblastine was added 24 hours before Compound K in a 5 day assay. Results are shown hereafter; see FIG. 29 and FIG. 30. Synergy up to 45% is observed at concentrations tested. CI=0.07.

Vinblastine was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 1.70E−07 | 5.10E−07 | 1.50E−06 | 4.60E−06 | 1.40E−05 | 4.10E−05 | 1.20E−04 | 0.00037 | 0.001 | 0.003 | 0.01 |
| 0 | 0.0 | 0.0 | 0.0 | 0.0 | 0.0 | 0.0 | 0.0 | 0.0 | 0.0 | 0.0 | −0.0 | 0.0 |
| 0.01 | 0.0 | −6.0 | −25.1 | 3.3 | −2.0 | −8.2 | 1.8 | 1.3 | 1.4 | 0.2 | −0.0 | −0.2 |
| 0.04 | 0.0 | 35.2 | 28.1 | 58.5 | 48.6 | 32.3 | 3.8 | 2.7 | 1.8 | 1.2 | 0.0 | 0.1 |
| 0.12 | 0.0 | 61.9 | 26.9 | 65.6 | 51.9 | 34.3 | 3.2 | 2.5 | 1.6 | 1.4 | 0.0 | −0.1 |
| 0.37 | 0.0 | 50.1 | 28.0 | 43.0 | 20.4 | 25.5 | 2.5 | 2.0 | 1.4 | 1.2 | −0.0 | −0.3 |
| 1.11 | 0.0 | 50.6 | 32.8 | 30.9 | 12.0 | 22.1 | 2.8 | 1.7 | 1.4 | 1.1 | −0.1 | −0.3 |
| 3.33 | 0.0 | 18.5 | 10.2 | 12.9 | 1.5 | 12.5 | 0.5 | 0.9 | 0.2 | 0.6 | −0.2 | −0.4 |
| 10 | 0.0 | −20.6 | 1.6 | 5.5 | 0.6 | 3.9 | 0.1 | −0.3 | −0.2 | −0.0 | −0.1 | 0.0 |
| 0 | 0.0 | 0.0 | 0.0 | 0.0 | 0.0 | 0.0 | 0.0 | 0.0 | 0.0 | 0.0 | 0.0 | 0.0 |
| 0.01 | 0.0 | 35.8 | 27.2 | 49.0 | 23.5 | 17.0 | 0.0 | 0.8 | 0.0 | 0.1 | −0.2 | −0.3 |
| 0.04 | 0.0 | 41.4 | 25.6 | 48.2 | 38.6 | 17.8 | −1.2 | 0.5 | −0.1 | −0.0 | −0.1 | −0.1 |
| 0.12 | 0.0 | 15.3 | 15.4 | 31.1 | 32.0 | 7.7 | 0.5 | 0.3 | −0.2 | −0.1 | −0.2 | −0.1 |
| 0.37 | 0.0 | 29.9 | 23.4 | 37.2 | 26.6 | 9.2 | 0.4 | 0.3 | −0.1 | 0.0 | −0.1 | 0.2 |
| 1.11 | 0.0 | 34.6 | 17.1 | 38.1 | 19.7 | 8.8 | 0.4 | 0.5 | −0.4 | 0.2 | −0.3 | −0.4 |
| 3.33 | 0.0 | 31.2 | 16.5 | 33.2 | 28.9 | 10.8 | 0.2 | 0.3 | −0.3 | −0.3 | −0.4 | 0.1 |
| 10 | 0.0 | 0.1 | −0.5 | 2.2 | 2.9 | −0.2 | −0.9 | −0.5 | −0.3 | −0.2 | −0.3 | −0.6 |

Compound K: IC50=4.1 uM, Top=10022 RFU

Vinblastine: IC50=14 pM, Top=9697 RFU

Value of 50% effect=4930 RFU

50% Effect was achieved by combining 120 nM Compound K and 0.5 pM Vinblastine.

CI=[Compound K]/IC50_{Compound K}+[Vinblastine]/IC50_{Vinblastine}=(0.12/4.1)+(0.5/14)=0.07

### Example 14

### Gemcitabine/Compound K Combination Testing in MIA PaCa-2 Pancreatic Cancer Cells

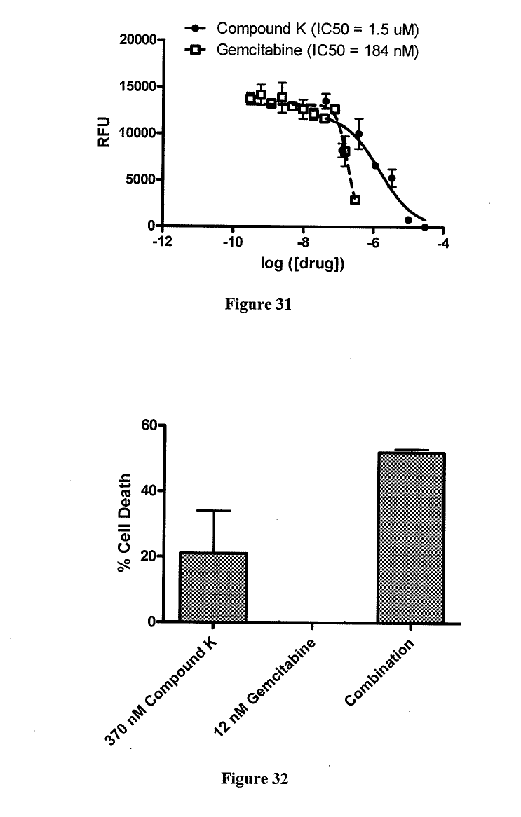

Gemcitabine, a pyrimidine analog, was tested in combination with Compound K in the pancreatic cancer cell line MIA PaCa-2. Gemcitabine was added 24 hours before Compound K in a 4 day assay. Results are shown hereafter; see FIG. 31 and FIG. 32. Synergy up to 25% is observed at concentrations tested. CI=0.27.

Gemcitabine was added first, Compound K the next day (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.0003 | 0.0006 | 0.0012 | 0.0023 | 0.0047 | 0.0094 | 0.019 | 0.0375 | 0.075 | 0.15 | 0.3 |
| 0 | 0.0 | −0.0 | 0.0 | 0.0 | −0.0 | −0.0 | 0.0 | 0.0 | 0.0 | 0.0 | 0.0 | 0.0 |
| 0.04 | 0.0 | 24.6 | 17.0 | 27.8 | 38.8 | 56.7 | 26.8 | 31.7 | 27.1 | 35.5 | 42.8 | 1.4 |
| 0.12 | 0.0 | 12.7 | 18.9 | 19.4 | 5.7 | 17.0 | 13.4 | 23.8 | 23.7 | 23.3 | 13.0 | −11.4 |
| 0.37 | 0.0 | 12.0 | 32.1 | 32.2 | 25.2 | 26.1 | 8.0 | 5.9 | 10.0 | 18.3 | 19.6 | −3.9 |
| 1.1 | 0.0 | 19.0 | 23.7 | 24.8 | 4.0 | 21.3 | 9.5 | 9.2 | 3.9 | 19.7 | 15.7 | −4.8 |
| 3.3 | 0.0 | 9.8 | 1.1 | 11.7 | 12.5 | 13.4 | 9.1 | 2.7 | 5.4 | 13.9 | 13.4 | −8.3 |
| 10 | 0.0 | −0.7 | 3.5 | 4.0 | 4.2 | 2.7 | 4.0 | 4.2 | 0.9 | 4.7 | 1.4 | −1.5 |
| 30 | 0.0 | 0.6 | 0.4 | 0.5 | 0.3 | 0.2 | 0.1 | −0.3 | −0.3 | 0.1 | −0.0 | −0.3 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | 35.8 | 27.2 | 49.0 | 23.5 | 17.0 | 0.0 | 0.8 | 0.0 | 0.1 | −0.2 | −0.3 |
| 0.12 | 0.0 | 41.4 | 25.6 | 48.2 | 38.6 | 17.8 | −1.2 | 0.5 | −0.1 | −0.0 | −0.1 | −0.1 |
| 0.37 | 0.0 | 15.3 | 15.4 | 31.1 | 32.0 | 7.7 | 0.5 | 0.3 | −0.2 | −0.1 | −0.2 | −0.1 |
| 1.1 | 0.0 | 29.9 | 23.4 | 37.2 | 26.6 | 9.2 | 0.4 | 0.3 | −0.1 | 0.0 | −0.1 | 0.2 |
| 3.3 | 0.0 | 34.6 | 17.1 | 38.1 | 19.7 | 8.8 | 0.4 | 0.5 | −0.4 | 0.2 | −0.3 | −0.4 |
| 10 | 0.0 | 31.2 | 16.5 | 33.2 | 28.9 | 10.8 | 0.2 | 0.3 | −0.3 | −0.3 | −0.4 | 0.1 |
| 30 | 0.0 | 0.1 | −0.5 | 2.2 | 2.9 | −0.2 | −0.9 | −0.5 | −0.3 | −0.2 | −0.3 | −0.6 |

Compound K: IC50=1.5 uM, Top=12202 RFU

Gemcitabine: IC50=184 nM, Top=13153 RFU

Value of 50% effect=6339 RFU

50% Effect was achieved by combining 370 nM Compound K and 12 nM Gemcitabine.

CI=[Compound K]/IC50_{Compound K}+[Gemcitabine]/IC50_{Gemcitabine}=(0.37/1.5)+(12/184)=0.27

### Example 15

### Sunitinib/Compound K Combination Testing in MIA PaCa-2 Pancreatic Cancer Cells

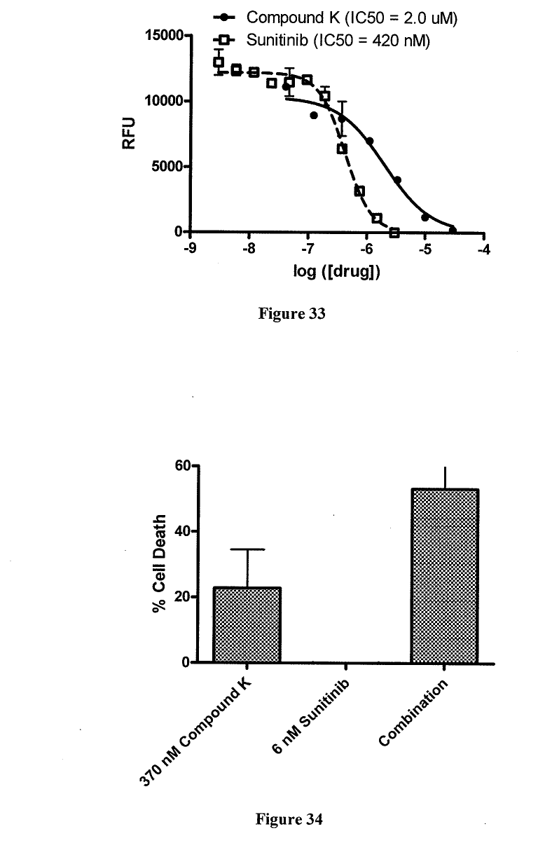

Sunitinib, a multi tyrosine-kinase inhibitor as described herein, was tested in combination with Compound K in the pancreatic cancer cell line MIA PaCa-2. Sunitinib was added 24 hours before Compound K in a 4 day assay. Results are shown hereafter; see FIG. 33 and FIG. 34. Synergy up to 25% is observed at concentrations tested. CI=0.2.

Sunitinib was added first, Compound K the next day (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.0029 | 0.0059 | 0.012 | 0.023 | 0.047 | 0.094 | 0.19 | 0.38 | 0.75 | 1.5 | 3 |
| 0 | 0.0 | 0.0 | −0.0 | −0.0 | 0.0 | 0.0 | 0.0 | 0.0 | 0.0 | 0.0 | 0.0 | 0.0 |
| 0.04 | 0.0 | 1.2 | −4.3 | 4.4 | 17.2 | 14.7 | 4.5 | −5.9 | −7.6 | −1.3 | −2.7 | −0.1 |
| 0.12 | 0.0 | 23.8 | 8.6 | 16.0 | 4.5 | 11.5 | 8.4 | −9.1 | −5.0 | 2.8 | −1.6 | −0.0 |
| 0.37 | 0.0 | 13.4 | −3.9 | 8.0 | 2.5 | 12.2 | −8.0 | −1.8 | −6.5 | −6.6 | −3.4 | −0.1 |
| 1.1 | 0.0 | −3.1 | −1.2 | 17.7 | 6.2 | 14.4 | 6.0 | 4.9 | −8.5 | −8.3 | −3.1 | −0.0 |
| 3.3 | 0.0 | 5.1 | 3.0 | 4.6 | 0.3 | 9.4 | 5.3 | 5.8 | −0.8 | −6.6 | −7.0 | −0.3 |
| 10 | 0.0 | −4.3 | −5.9 | −1.3 | 0.5 | −4.4 | −0.4 | −0.9 | −0.6 | −3.1 | −2.6 | −0.1 |
| 30 | 0.0 | −0.3 | −0.5 | −0.1 | −0.2 | −0.4 | −0.2 | −0.9 | −1.4 | −2.0 | −1.5 | 0.0 |
| 0 | 0.0 | 0.0 | 0.0 | 0.0 | 0.0 | 0.0 | 0.0 | 0.0 | 0.0 | 0.0 | 0.0 | 0.0 |
| 0.04 | 0.0 | 10.6 | 13.8 | 22.5 | 28.9 | 4.2 | 32.8 | 39.8 | 12.4 | 8.9 | 4.3 | −0.2 |
| 0.12 | 0.0 | 18.1 | 18.0 | 12.7 | 16.0 | 10.6 | 15.0 | 19.9 | 17.5 | 1.7 | 3.5 | −0.3 |
| 0.37 | 0.0 | 44.2 | 52.0 | 37.9 | 35.2 | 37.7 | 41.7 | 32.2 | 13.7 | 14.4 | 2.3 | −0.2 |
| 1.1 | 0.0 | 19.3 | 26.1 | 9.2 | 19.0 | 12.2 | 24.4 | 21.8 | 7.3 | −5.3 | 1.6 | −0.2 |
| 3.3 | 0.0 | −1.2 | 8.3 | 19.6 | 4.7 | 4.1 | −9.8 | 7.4 | 0.5 | −6.3 | −4.3 | −0.3 |
| 10 | 0.0 | 5.2 | 0.5 | 2.0 | 3.3 | 2.3 | 2.7 | 4.3 | −0.9 | −3.2 | −1.0 | −0.2 |
| 30 | 0.0 | 0.0 | −0.0 | −4.0 | −0.4 | −0.6 | −0.1 | −0.7 | −1.6 | −2.1 | −0.9 | −0.1 |

Compound K: IC50=2.0 uM, Top=10345 RFU

Sunitinib: IC50=420 nM, Top=12195 RFU

Value of 50% effect=5635 RFU

50% Effect was achieved by combining 370 nM Compound K and 6 nM Sunitinib.

CI=[Compound K]/IC50_{Compound K}+[Sunitinib]/IC50_{Sunitinib}=(0.37/1.5)+(12/184)=0.27

### Example 16

### Rapamycin/Compound K Combination Testing in MIA PaCa-2 Pancreatic Cancer cells

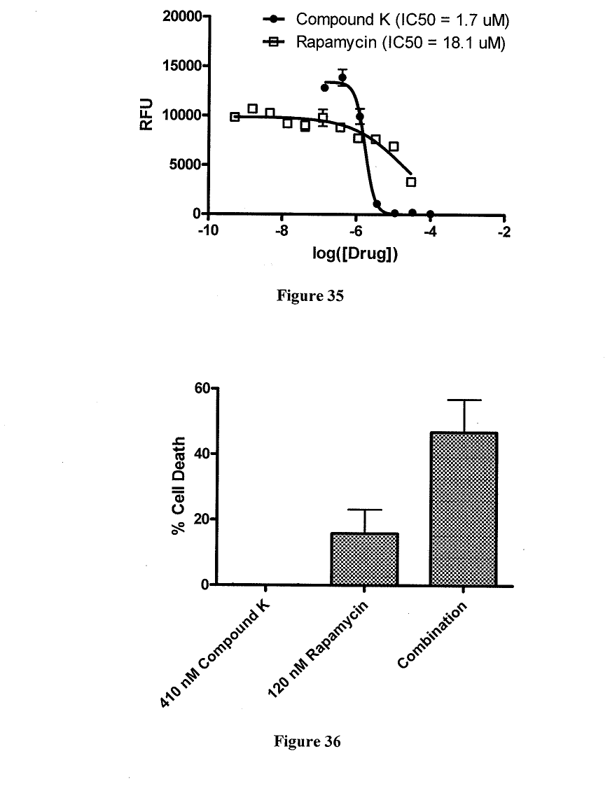

Rapamycin, an immunosuppressive macrolide, was tested in combination with Compound K in the pancreatic cancer cell line MIA PaCa-2. Rapamycin and Compound K are added simultaneously in a 4 day assay. Results are shown hereafter; see FIG. 35 and FIG. 36. Synergy up to 30% is observed at concentrations tested. CI=0.25.

Rapamycin and Compound K are added simultaneously (4 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.0005 | 0.0015 | 0.0046 | 0.014 | 0.041 | 0.12 | 0.37 | 1.1 | 3.3 | 10 | 30 |
| 0 | 0.0 | 0.0 | 0.0 | 0.0 | 0.0 | 0.0 | 0.0 | 0.0 | 0.0 | 0.0 | 0.0 | 0.0 |
| 0.14 | 0.0 | 1.4 | 16.7 | 1.2 | 9.5 | 9.4 | 25.8 | 10.5 | 3.9 | 8.9 | 4.8 | −5.9 |
| 0.41 | 0.0 | 10.7 | 35.4 | 28.4 | 22.0 | 24.4 | 41.8 | 23.0 | 20.9 | 18.6 | 16.9 | −1.3 |
| 1.2 | 0.0 | 13.9 | 22.0 | 15.9 | 19.4 | 21.9 | 29.0 | 19.4 | 15.7 | 14.4 | 12.7 | 5.4 |
| 3.7 | 0.0 | −1.7 | −0.8 | −0.2 | −2.1 | −0.8 | −0.2 | −1.1 | −2.0 | −1.3 | 0.8 | 0.5 |
| 11.1 | 0.0 | −0.4 | −0.4 | −0.3 | −0.5 | −0.3 | −0.3 | −0.5 | −0.6 | −0.3 | −0.8 | −0.6 |
| 33.3 | 0.0 | −0.4 | −0.6 | −0.7 | −0.9 | −0.9 | −0.5 | −0.7 | −1.0 | −1.1 | −0.7 | −0.1 |
| 100 | 0.0 | −0.4 | −1.0 | −1.5 | −0.4 | −0.6 | −1.9 | −1.6 | −0.7 | −0.1 | −0.9 | −0.3 |
| 0 | 0.0 | −0.0 | 0.0 | 0.0 | 0.0 | 0.0 | 0.0 | 0.0 | 0.0 | 0.0 | 0.0 | 0.0 |
| 0.14 | 0.0 | 7.5 | 15.0 | 13.5 | 3.9 | 9.0 | 3.4 | 16.5 | 20.8 | 17.8 | 3.5 | −11.1 |
| 0.41 | 0.0 | 10.5 | 16.1 | 22.3 | 13.1 | 4.1 | 12.7 | 12.8 | 19.2 | 17.6 | 10.1 | 0.9 |
| 1.2 | 0.0 | 29.2 | 36.0 | 36.5 | 29.6 | 25.9 | 32.2 | 32.5 | 34.1 | 34.4 | 24.6 | 11.0 |
| 3.7 | 0.0 | 1.5 | 1.7 | 0.9 | 1.0 | −0.0 | 0.4 | 0.5 | 0.4 | 0.1 | 0.2 | 0.3 |
| 11.1 | 0.0 | −0.2 | −0.4 | −0.1 | −0.3 | −0.4 | −0.6 | −0.4 | −0.3 | −0.5 | −0.6 | −0.3 |
| 33.3 | 0.0 | −0.1 | −0.3 | −0.2 | −0.6 | −0.5 | −0.6 | −0.6 | −0.6 | −0.8 | −0.5 | −0.2 |
| 100 | 0.0 | 0.4 | −2.3 | 0.5 | 0.5 | −0.0 | −0.4 | −0.0 | 0.1 | 0.6 | 0.5 | 0.3 |

Compound K: IC50=1.7 uM, Top=13393 RFU

Rapamycin: IC50=18.1 uM, Top=9864 RFU

Value of 50% effect=5814 RFU

50% Effect was achieved by combining 410 nM Compound K and 120 nM Rapamycin.

CI=[Compound K]/IC50_{Compound K}+F[Rapamycin]/IC50_{Rapamycin}=(0.41/1.7)+(0.12/18.1)=0.25

### Example 17

### 5-Fluorouracil/Compound K Combination Testing in SUM-149PT Inflammatory Breast Carcinoma Cells

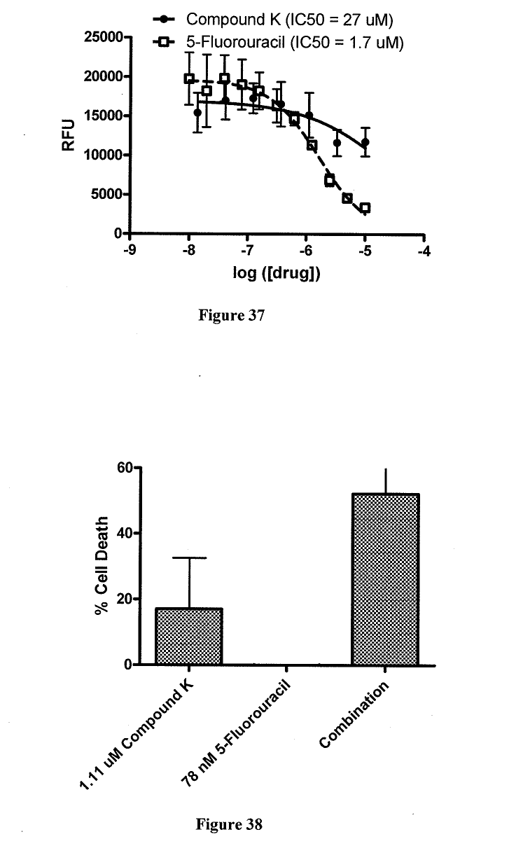

5-Fluorouracil, a pyrimidine analog, was tested in combination with Compound K in the inflammatory breast carcinoma cell line SUM-149PT. 5-Fluorouracil was added 24 hours before Compound K in a 5 day assay. Results are shown hereafter; see FIG. 37 and FIG. 38. Synergy up to 30% is observed at concentrations tested. CI=0.09.

5-Fluorouracil was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.0098 | 0.0195 | 0.039 | 0.078 | 0.16 | 0.31 | 0.63 | 1.3 | 2.5 | 5 | 10 |
| 0 | 0.0 | 0.0 | 0.0 | 0.0 | 0.0 | 0.0 | 0.0 | 0.0 | 0.0 | 0.0 | 0.0 | 0.0 |
| 0.01 | 0.0 | −5.0 | −5.8 | −8.0 | 10.9 | 1.4 | −4.9 | −9.9 | −0.5 | −6.3 | −2.0 | −2.0 |
| 0.04 | 0.0 | 13.0 | 4.7 | 10.3 | 44.7 | 25.4 | 19.1 | 19.9 | 10.5 | 3.4 | 7.5 | 5.5 |
| 0.12 | 0.0 | −1.7 | 8.8 | −1.5 | 23.7 | 15.5 | 10.9 | 9.6 | 0.1 | 5.5 | 7.6 | 3.4 |
| 0.37 | 0.0 | 0.4 | 38.3 | 8.8 | 29.4 | 26.3 | 13.7 | 14.1 | 11.6 | 8.5 | 8.1 | 2.7 |
| 1.11 | 0.0 | 5.2 | 9.1 | 5.5 | 30.4 | 21.8 | 21.7 | 22.5 | 15.5 | 14.2 | 9.1 | 6.9 |
| 3.33 | 0.0 | 7.5 | 8.6 | 14.3 | 18.5 | 18.6 | 20.6 | 12.4 | 9.3 | 9.3 | 9.7 | 7.5 |
| 10 | 0.0 | 6.9 | 0.7 | 7.8 | 8.5 | 9.4 | 8.4 | 15.3 | 12.1 | 10.7 | 10.8 | 9.4 |
| 0 | 0.0 | 0.0 | 0.0 | 0.0 | 0.0 | −0.0 | 0.0 | 0.0 | 0.0 | 0.0 | 0.0 | 0.0 |
| 0.01 | 0.0 | −3.5 | −3.0 | −0.5 | 2.9 | 1.4 | −0.6 | 2.9 | −2.6 | −18.0 | −9.5 | −6.8 |
| 0.04 | 0.0 | 20.5 | 22.7 | 30.3 | 24.7 | 26.7 | 11.1 | 10.5 | 6.5 | −10.1 | 6.5 | −7.2 |
| 0.12 | 0.0 | 24.3 | 26.8 | 41.1 | 26.5 | 33.9 | 18.1 | 25.7 | 14.6 | 1.0 | 1.6 | 1.7 |
| 0.37 | 0.0 | 27.7 | 18.1 | 28.1 | 30.6 | 25.7 | 15.4 | 19.6 | 13.9 | −3.7 | 5.3 | 0.4 |
| 1.11 | 0.0 | 20.9 | 16.6 | 21.7 | 29.0 | 19.5 | 12.5 | 12.7 | 11.8 | 6.9 | 8.1 | 4.3 |
| 3.33 | 0.0 | 17.3 | 17.2 | 24.5 | 25.5 | 22.0 | 16.3 | 17.4 | 11.2 | 7.2 | 12.1 | 10.4 |
| 10 | −0.0 | −1.2 | −2.7 | 8.9 | 11.1 | 10.9 | 5.3 | 22.5 | 19.8 | 13.2 | 14.9 | 11.7 |

Compound K: 1050=27 uM, Top=17000 RFU

5-Fluorouracil: 1050=1.7 uM, Top=19618 RFU

Value of 50% effect=9154 RFU

50% Effect was achieved by combining 1.11 uM Compound K and 78 nM 5-Fluorouracil.

CI=[Compound K]/IC50_{Compound K}+[5-Fluorouracil]/IC50_{5-Fluorouracil}=(1.11/27)+(0.078/1.7)=0.09

### Example 18

### Cisplatin/Compound K Combination Testing in SUM-149PT Inflammatory Breast Carcinoma Cells

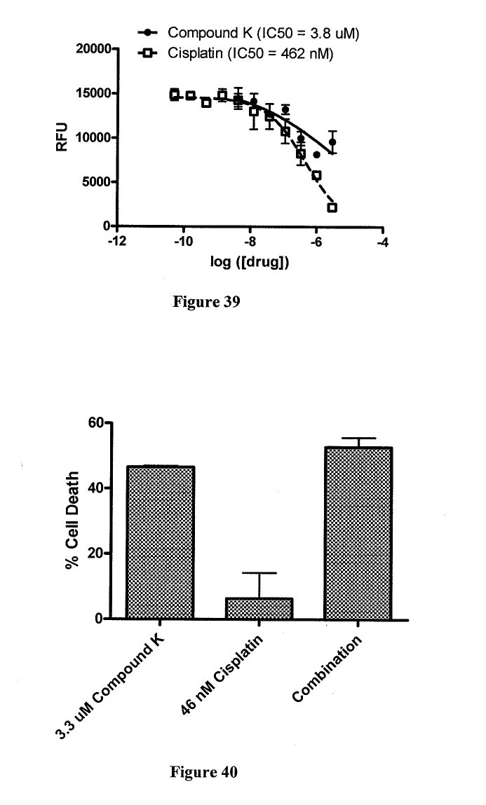

Cisplatin, an alkylating-like agent, was tested in combination with Compound K in the inflammatory breast carcinoma cell line SUM-149PT. Cisplatin was added 24 hours before Compound K in a 5 day assay. Results are shown hereafter; see FIG. 39 and FIG. 40. Synergy up to 25% is observed at concentrations tested. CI=0.88.

Cisplatin was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.0002 | 0.0005 | 0.0015 | 0.0046 | 0.014 | 0.041 | 0.12 | 0.37 | 1.1 | 3 | 10 |
| 0 | 0.0 | 0.0 | 0.0 | 0.0 | 0.0 | 0.0 | 0.0 | 0.0 | 0.0 | 0.0 | 0.0 | 0.0 |
| 0.01 | 0.0 | 8.1 | 14.2 | 19.3 | 11.6 | 20.3 | 14.5 | 10.5 | 7.8 | −3.0 | 9.3 | −1.0 |
| 0.04 | 0.0 | 14.8 | 17.2 | 18.8 | 14.9 | 8.3 | −3.5 | 4.7 | −9.1 | −14.4 | 2.5 | −2.2 |
| 0.12 | 0.0 | 8.5 | 3.7 | 12.5 | 10.3 | 6.4 | −9.6 | 0.8 | −9.4 | −13.8 | 5.3 | 1.9 |
| 0.37 | 0.0 | 1.8 | 16.9 | 14.5 | 22.3 | 11.9 | −13.4 | 3.8 | 0.1 | −10.2 | 9.3 | 0.4 |
| 1.11 | 0.0 | −11.9 | −1.5 | −6.7 | −5.9 | −11.2 | −20.6 | −11.2 | −24.1 | −15.5 | 2.3 | 1.2 |
| 3.33 | 0.0 | 3.9 | 13.0 | 9.5 | 10.3 | 0.2 | −14.2 | −6.4 | −17.0 | −3.4 | 10.1 | 4.5 |
| 10 | 0.0 | 26.2 | 20.1 | 26.3 | 28.3 | 23.0 | 11.5 | 10.0 | 19.6 | 14.8 | 25.5 | 8.8 |
| 0 | 0.0 | 0.0 | 0.0 | −0.0 | 0.0 | 0.0 | 0.0 | 0.0 | 0.0 | 0.0 | 0.0 | 0.0 |
| 0.01 | 0.0 | 23.6 | 24.3 | 20.0 | 29.0 | 16.1 | 31.2 | 19.8 | −2.7 | 5.8 | 1.4 | 1.4 |
| 0.04 | 0.0 | 41.9 | 24.0 | 30.1 | 16.4 | 25.1 | 34.8 | 15.4 | −1.0 | −10.6 | −0.5 | −2.8 |
| 0.12 | 0.0 | 13.2 | 14.1 | 8.7 | 6.6 | 15.1 | 17.1 | 9.6 | 1.9 | −10.0 | 1.8 | −2.2 |
| 0.37 | 0.0 | 29.0 | 25.9 | 12.0 | 29.0 | 16.7 | 21.9 | 20.2 | 7.3 | 1.3 | 8.8 | −0.7 |
| 1.11 | 0.0 | 19.7 | 12.5 | −3.7 | 8.3 | 6.9 | −3.1 | 0.8 | −10.2 | −2.4 | 4.4 | 0.7 |
| 3.33 | 0.0 | 12.8 | 2.1 | 5.0 | 2.3 | −10.9 | −3.3 | −9.2 | −7.8 | 3.8 | 7.2 | 2.1 |
| 10 | 0.0 | 3.3 | 4.5 | 3.7 | 9.7 | −2.8 | 0.9 | 7.8 | 6.5 | 7.7 | 12.3 | 4.7 |

Compound K: 1050=3.8 uM, Top=16000 RFU

Cisplatin: 1050=462 nM, Top=14588 RFU

Value of 50% effect=7547 RFU

50% Effect was achieved by combining 3.3 uM Compound K and 46 nM Cisplatin.

CI=[Compound K]/IC50_{Compound K}+[Cisplatin]/IC50_{Cisplatin}=(3.3/3.8)+(46/462)=0.88

### Example 19

### Rapamycin/Compound K Combination Testing in SUM-149PT Inflammatory Breast Carcinoma Cells

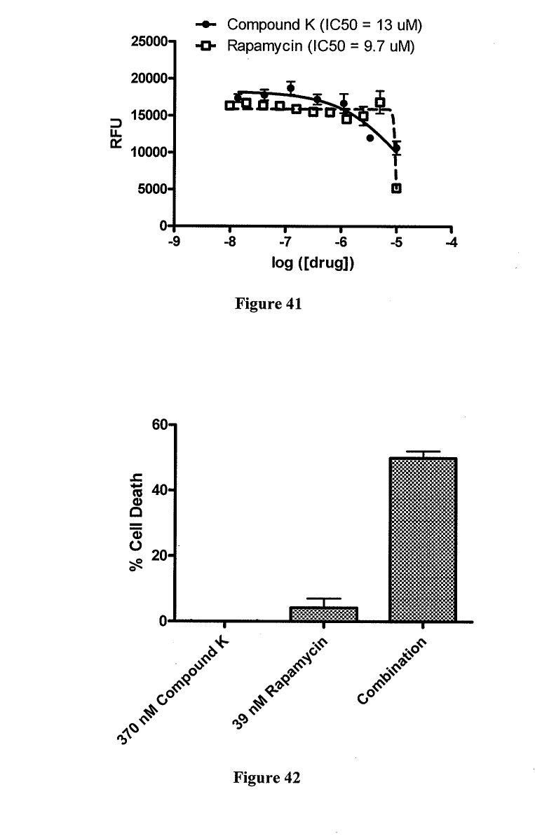

Rapamycin, an immunosuppressive macrolide, was tested in combination with Compound K in the inflammatory breast carcinoma cell line SUM-149PT Rapamycin was added 24 hours before Compound K in a 5 day assay. Results are shown hereafter; see FIG. 41 and FIG. 42. Synergy up to 40% is observed at concentrations tested. CI=0.03.

Rapamycin was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.010 | 0.020 | 0.039 | 0.078 | 0.16 | 0.31 | 0.63 | 1.3 | 2.5 | 5 | 10 |
| 0 | 0.0 | 0.0 | 0.0 | 0.0 | 0.0 | 0.0 | 0.0 | 0.0 | 0.0 | 0.0 | 0.0 | 0.0 |
| 0.01 | 0.0 | −3.5 | 7.9 | 28.5 | 15.8 | 39.1 | 25.0 | 23.7 | −11.0 | −5.0 | −11.5 | −4.9 |
| 0.04 | 0.0 | 29.3 | 22.0 | 22.2 | 23.8 | 25.2 | 34.3 | 32.7 | −1.0 | −19.7 | −10.1 | −2.2 |
| 0.12 | 0.0 | 43.5 | 47.9 | 54.0 | 46.1 | 50.2 | 51.0 | 44.7 | 32.3 | 15.5 | −3.3 | 0.7 |
| 0.37 | 0.0 | 42.9 | 48.0 | 46.6 | 42.4 | 40.8 | 43.1 | 39.2 | 24.2 | −4.8 | −10.1 | 4.2 |
| 1.11 | 0.0 | 33.2 | 39.7 | 32.8 | 42.1 | 34.3 | 39.3 | 36.3 | 16.3 | −7.5 | 16.2 | 5.1 |
| 3.33 | 0.0 | 10.4 | 36.3 | 35.9 | 31.8 | 34.7 | 33.0 | 30.9 | 27.8 | 26.5 | 32.6 | 2.3 |
| 10 | 0.0 | 2.9 | 5.2 | 5.6 | 4.4 | 4.6 | 2.7 | 6.0 | 1.4 | −1.4 | 18.0 | 3.0 |
| 0 | 0.0 | 0.0 | −0.0 | 0.0 | 0.0 | 0.0 | 0.0 | 0.0 | 0.0 | −0.0 | 0.0 | 0.0 |
| 0.01 | 0.0 | 2.5 | 1.0 | 2.1 | 8.1 | −5.6 | −14.0 | −2.6 | −3.6 | 10.0 | −9.8 | 3.1 |
| 0.04 | 0.0 | 29.7 | 28.2 | 31.6 | 35.6 | 28.2 | 25.2 | 25.8 | 19.7 | −10.4 | −13.8 | −3.2 |
| 0.12 | 0.0 | 25.6 | 20.6 | 35.8 | 38.6 | 30.7 | 31.8 | 22.7 | 18.4 | 21.0 | −10.1 | 3.2 |
| 0.37 | 0.0 | 27.6 | 25.7 | 29.1 | 30.3 | 28.4 | 23.3 | 16.0 | 5.8 | 1.9 | −15.0 | 1.6 |
| 1.11 | 0.0 | 26.8 | 30.2 | 27.9 | 36.4 | 25.9 | 20.2 | 24.2 | −2.4 | 27.1 | 22.4 | 4.2 |
| 3.33 | 0.0 | 16.5 | 24.2 | 22.1 | 30.8 | 15.2 | 6.3 | 16.7 | 11.0 | 15.6 | 25.6 | −3.8 |
| 10 | 0.0 | −3.0 | −9.1 | −11.1 | −12.0 | −10.1 | −6.0 | −9.4 | −10.1 | −3.4 | 13.7 | −5.5 |

Compound K: IC50=13 uM, Top=18285 RFU

Rapamycin: IC50=9.7 uM, Top=15915 RFU

Value of 50% effect=8550 RFU

50% Effect was achieved by combining 370 nM Compound K and 39 nM Rapamycin.

CI=[Compound K]/IC50_{Compound K}+[Rapamycin]/IC50_{Rapamycin}=(0.37/13)+(0.039/9.7)=0.03

### Example 20

### Erlotinib/Compound K Combination Testing in SUM-149PT Inflammatory Breast carcinoma cells

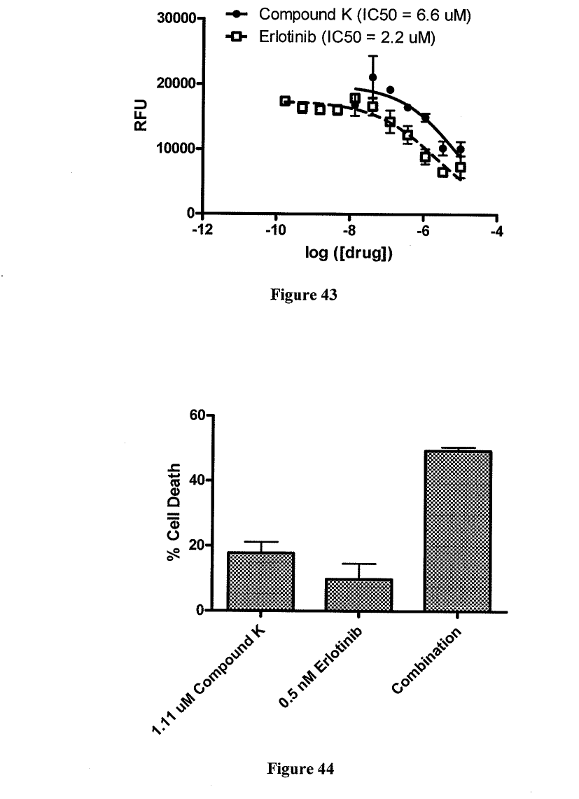

Erlotinib, a small molecule EGFR inhibitor, was tested in combination with Compound K in the inflammatory breast carcinoma cell line SUM-149PT. Erlotinib was added 24 hours before Compound K in a 5 day assay. Results are shown hereafter; see FIG. 43 and FIG. 44. Synergy up to 35% is observed at concentrations tested. CI=0.16.

Erlotinib was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00017 | 0.00051 | 0.0015 | 0.0046 | 0.014 | 0.041 | 0.12 | 0.37 | 1.1 | 3.3 | 10 |
| 0 | 0.0 | −0.0 | 0.0 | 0.0 | 0.0 | 0.0 | 0.0 | 0.0 | 0.0 | 0.0 | 0.0 | 0.0 |
| 0.01 | 0.0 | −7.3 | −6.5 | −7.6 | −9.0 | 3.3 | 27.7 | 5.1 | −2.4 | −18.1 | −10.8 | −11.7 |
| 0.04 | 0.0 | −7.3 | 41.6 | 18.9 | 28.3 | 35.1 | 13.8 | 32.7 | 15.3 | −2.5 | −8.0 | −4.5 |
| 0.12 | 0.0 | −7.1 | 28.3 | 26.0 | 36.7 | 41.4 | 34.4 | 11.4 | 5.1 | −14.4 | −9.4 | −11.3 |
| 0.37 | 0.0 | −5.3 | 39.8 | 19.5 | 30.0 | 40.8 | 43.9 | 36.0 | 15.1 | −17.3 | −7.8 | 24.4 |
| 1.11 | 0.0 | −4.2 | 23.8 | 13.7 | 27.3 | 31.0 | 21.8 | 31.6 | 6.8 | −8.8 | −14.7 | −15.4 |
| 3.33 | 0.0 | 6.5 | 18.1 | 9.0 | 11.3 | 9.5 | 1.7 | 6.2 | −1.3 | −9.9 | −20.0 | −19.1 |
| 10 | 0.0 | −0.0 | −2.4 | −2.4 | −5.1 | 1.5 | 2.5 | 3.0 | −2.5 | −22.1 | −25.6 | −29.4 |
| 0 | 0.0 | 0.0 | 0.0 | 0.0 | 0.0 | 0.0 | 0.0 | 0.0 | −0.0 | 0.0 | 0.0 | 0.0 |
| 0.01 | 0.0 | 11.5 | 23.2 | 29.7 | 25.2 | 10.3 | 17.5 | 8.4 | 1.1 | 5.1 | −11.5 | 6.0 |
| 0.04 | 0.0 | −8.3 | 13.4 | 18.8 | 9.5 | 24.1 | 9.1 | 11.1 | 12.5 | 17.6 | −4.4 | 13.7 |
| 0.12 | 0.0 | −5.0 | 8.1 | 17.1 | 34.3 | 27.1 | 16.1 | 31.0 | 21.8 | 31.4 | 3.3 | 11.4 |
| 0.37 | 0.0 | −3.7 | 16.7 | 17.5 | 13.7 | 35.9 | 17.0 | 25.9 | 22.5 | 18.9 | 3.0 | 10.3 |
| 1.11 | 0.0 | 8.7 | 19.2 | 10.2 | 27.4 | 39.2 | 25.7 | 17.1 | 15.4 | 20.1 | 0.4 | 11.8 |
| 3.33 | 0.0 | 10.0 | 9.2 | 5.9 | 9.8 | 9.0 | 11.7 | 6.5 | −1.9 | 5.4 | −9.1 | −4.5 |
| 10 | 0.0 | 6.5 | 6.7 | 8.3 | −4.5 | 19.1 | 10.1 | −1.5 | −7.3 | −1.7 | −11.8 | −7.2 |

Compound K: IC50=6.9 uM, Top=19848 RFU

Erlotinib: IC50=2.2 uM, Top=17378 RFU

Value of 50% effect=9307 RFU

50% Effect was achieved by combining 1.1 uM Compound K and 0.5 nM Erlotinib.

CI=[Compound K]/IC50_{Compound K}+[Erlotinib]/IC50_{Erlotinib}=(1.11/6.9)+(0.00051/2.2)=0.16

### Example 21

### 5-Fluorouracil/Compound K Combination Testing in SUM-190PT Inflammatory Breast Carcinoma Cells

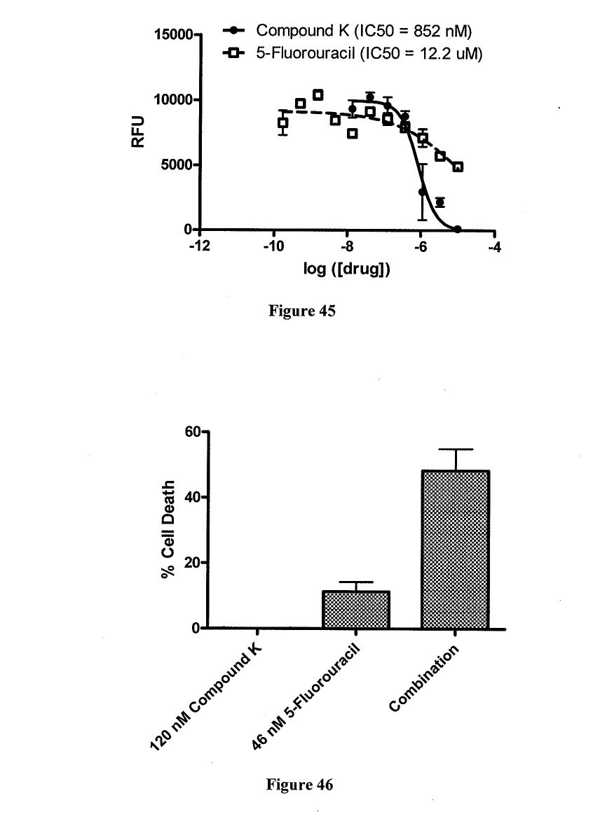

5-Fluorouracil, a pyrimidine analog, was tested in combination with Compound K in the inflammatory breast carcinoma cell line SUM-190PT. 5-Fluorouracil was added 24 hours before Compound K in a 5 day assay. Results are shown hereafter; see FIG. 45 and FIG. 46. Synergy up to 30% is observed at concentrations tested. CI=0.14.

5-Fluorouracil was added first, Compound K the next day (5 day assay in total). Results indicate the degree of inhibitory effect found with agent combination, where a positive value denotes synergy and a negative value antagonism. The experiment was performed in duplicate. Both data sets are presented.

| Conc. | Conc. Drug μM | | | | | | | | | | | |
|---|---|---|---|---|---|---|---|---|---|---|---|---|
| K μM | 0 | 0.00017 | 0.00051 | 0.0015 | 0.0046 | 0.014 | 0.041 | 0.12 | 0.37 | 1.1 | 3.3 | 10 |
| 0 | 0.0 | 0.0 | 0.0 | 0.0 | 0.0 | 0.0 | −0.0 | 0.0 | 0.0 | 0.0 | 0.0 | −0.0 |
| 0.01 | 0.0 | −14.6 | −8.8 | 0.9 | −18.9 | −27.7 | −14.6 | 9.6 | 45.6 | 20.5 | 9.1 | 7.2 |
| 0.04 | 0.0 | −15.7 | 41.2 | 19.5 | 36.3 | 24.9 | 31.6 | 41.2 | 35.7 | 15.4 | −21.9 | −6.0 |
| 0.12 | 0.0 | −12.4 | 41.4 | 37.4 | 29.5 | 50.7 | 39.3 | 35.2 | 32.9 | 26.6 | −19.0 | −4.7 |
| 0.37 | 0.0 | −13.1 | 50.3 | 33.4 | 34.9 | 25.3 | 34.3 | 31.4 | 31.9 | 21.0 | −23.7 | −6.4 |
| 1.11 | 0.0 | −2.9 | −1.5 | −9.4 | −14.4 | −2.7 | −10.9 | −2.8 | −4.2 | −9.7 | −30.1 | −22.8 |
| 3.33 | 0.0 | −6.9 | −4.9 | −4.4 | −2.0 | −2.6 | 12.3 | 5.6 | 11.3 | 9.2 | −12.1 | −7.0 |
| 10 | 0.0 | −1.4 | −0.8 | −2.1 | −2.2 | −2.0 | −2.9 | −2.4 | −3.1 | −4.2 | −3.2 | 0.5 |
| 0 | 0.0 | 0.0 | 0.0 | 0.0 | 0.0 | 0.0 | 0.0 | 0.0 | 0.0 | 0.0 | −0.0 | 0.0 |
| 0.01 | 0.0 | −41.4 | −22.5 | −19.5 | −34.3 | −33.3 | −18.5 | 17.0 | 19.2 | 14.5 | −0.8 | −0.3 |
| 0.04 | 0.0 | −29.7 | 31.3 | 29.0 | 10.0 | 18.9 | 25.8 | 25.7 | 20.1 | 19.8 | −11.7 | 2.3 |
| 0.12 | 0.0 | −9.5 | 33.5 | 35.7 | 26.2 | 19.5 | 28.8 | 32.4 | 30.6 | 19.9 | 11.0 | −0.6 |
| 0.37 | 0.0 | −43.5 | 29.0 | 41.1 | 23.6 | 21.9 | 32.6 | 27.9 | 23.0 | 17.0 | 11.0 | −7.5 |
| 1.11 | 0.0 | −13.3 | 16.0 | 25.6 | 11.8 | 9.9 | 13.3 | 21.5 | 11.1 | 7.9 | 0.9 | −10.0 |
| 3.33 | 0.0 | −8.9 | −1.7 | 0.9 | −1.2 | −1.1 | −0.5 | 2.6 | 3.4 | 3.8 | −5.8 | −8.3 |
| 10 | 0.0 | 0.8 | −0.1 | −0.2 | −0.2 | −0.6 | −1.1 | −0.9 | −1.0 | −0.5 | −1.6 | −1.9 |

Compound K: IC50=852 nM, Top=9958 RFU

5-Fluorouracil: 1050=12.2 uM, Top=9141 RFU

Value of 50% effect=4775 RFU

50% Effect was achieved by combining 120 nM Compound K and 46 nM 5-Fluorouracil.

CI=[Compound K]/IC50_{Compound K}+[5-Fluorouracil]/IC50_{5-Fluorouracil}=(120/852)+(0.046/12.2)=0.14

### Example 22

### Erlotinib/Compound K Combination Testing in Erlotinib-Sensitive BT-474 Breast Carcinoma Cells

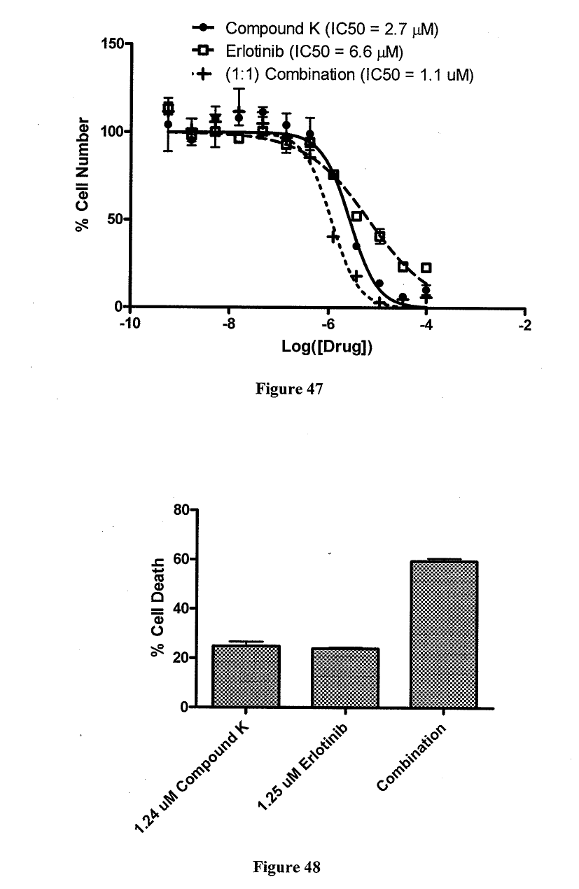

Erlotinib, a small molecule EGFR inhibitor, was tested in combination with Compound K in the breast carcinoma cell line BT-474. Erlotinib was added simultaneously with Compound K in a 4 day assay. Results are shown hereafter; see FIG. 47 and FIG. 48. Synergy was observed with CI=0.55.

Erlotinib was added simultaneously with Compound K in 1:1 ratio in a 4 day assay. The experiment was performed in triplicate. The dose-response curves for single agents and combination are presented.

Compound K: IC50=2.7 uM

Erlotinib: IC50=6.6 uM

About 60% effect was achieved by combining 1.24 nM Compound K and 1.25 uM erlotinib.

CI[IC50_{Combination}]/IC50_{Compound K}+[IC50_{Combination}]/IC⁵⁰_{Erlotinib}=(1.1/2.7)+(1.1/6.6)=0.57.

### Example 23

### Erlotinib/Compound K Combination Testing in Erlotinib-Resistant MDA-MB-453 Breast Carcinoma Cells

Erlotinib, a small molecule EGFR inhibitor, was tested in combination with Compound K in the breast carcinoma cell line MDA-MB453. Erlotinib was added simultaneously with Compound K in a 4 day assay. Results are shown hereafter. Synergy was observed with CI=0.55.

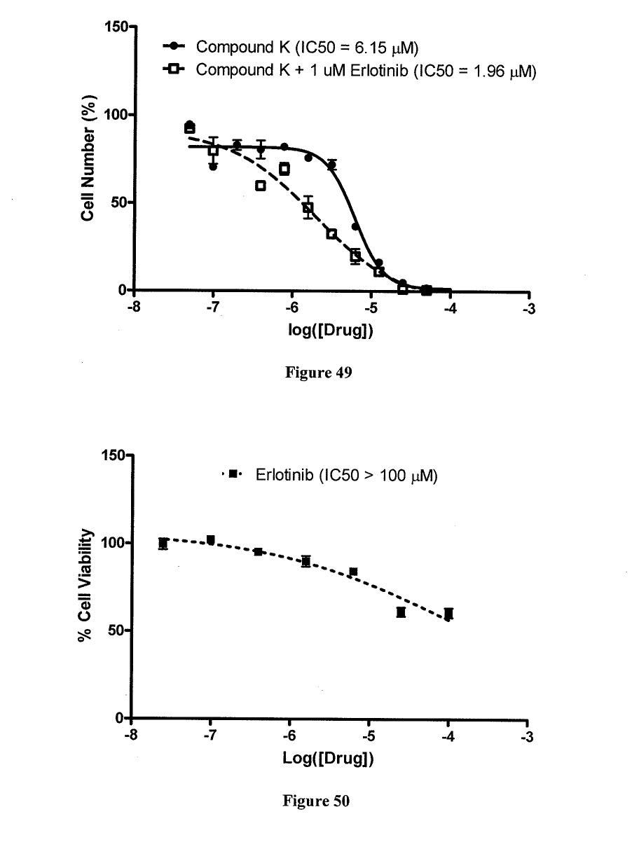

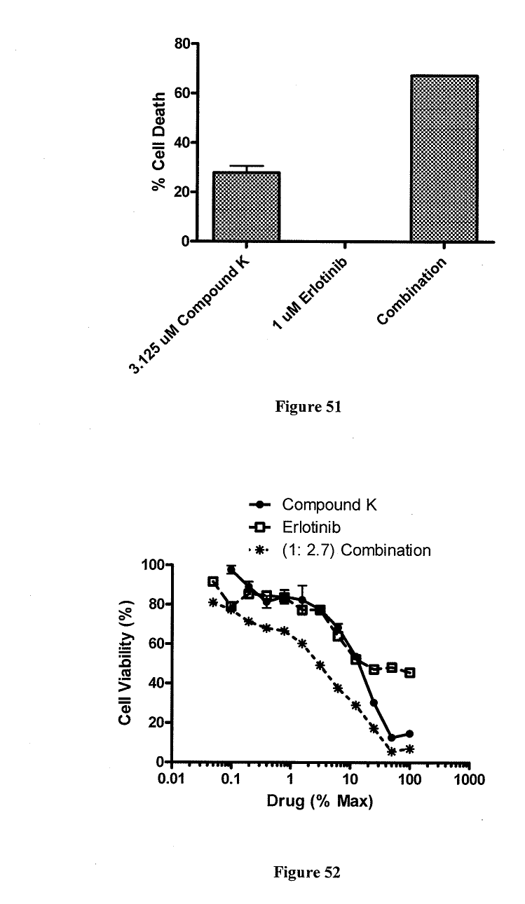

Compound K alone or in combination with 1 uM erlotinib was added to cells in a 4 day assay. The experiment was performed in triplicate. The dose-response curves for Compound K and combination are presented; see FIG. 49 and FIG. 50.

Compound K: IC50=6.15 uM

Erlotinib: IC50>100 uM

Greater than 60% effect was achieved by combining 3.125 uM Compound K and 1 uM erlotinib.

Combination 1050 Shift=6.15/1.96=3.14.

### Example 24

### Erlotinib/Compound K Combination Testing in Erlotinib-Resistant T47D Breast Carcinoma Cells

Erlotinib, a small molecule EGFR inhibitor, was tested in combination with Compound K in the breast carcinoma cell line T47D. Erlotinib was added simultaneously with Compound K in 1:2.7 ratio combination a 4 day assay. Results are shown hereafter. Synergy was observed with CI=0.48.

Erlotinib was added simultaneously with Compound K in 1:1 ratio in a 4 day assay. The experiment was performed in triplicate. The dose-response curves for single agents and combination are presented; see FIG. 52.

Compound K: 1050=5.9 uM; Maximum Concentration=37.5 uM

Erlotinib: IC50=47 uM; Maximum Concentration=100 uM

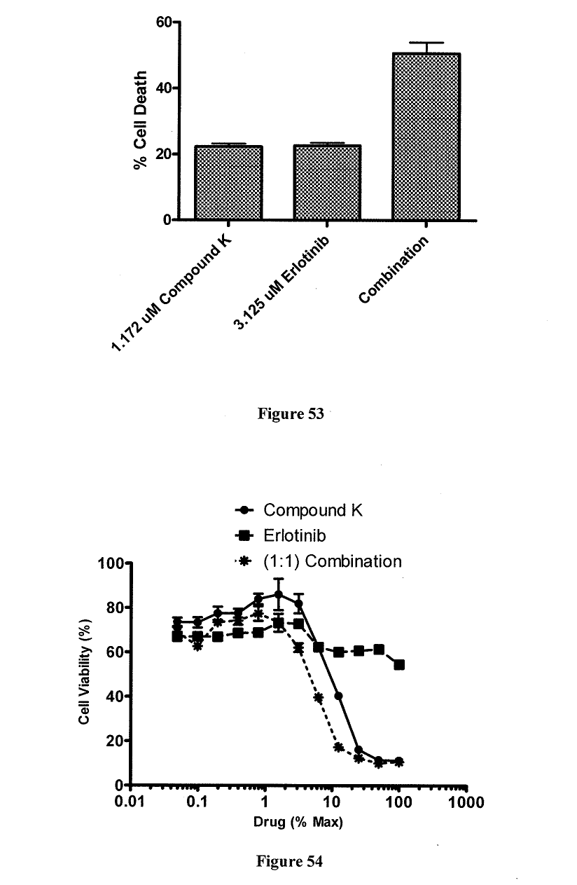

Combination: 50% Cell Death at 2.1 uM Compound K plus 5.7 uM Erlotinib; see FIG. 53.

CI=[IC50_{Combination}]/IC50_{Compound K}+[IC50_{Combination}]/IC⁵⁰_{Erlotinib}=(2.1/5.9)+(5.7/47)=0.48.

### Example 25

### Erlotinib/Compound K Combination Testing in Erlotinib-Resistant ZR-75-1 Breast Carcinoma Cells

Erlotinib, a small molecule EGFR inhibitor, was tested in combination with Compound K in the breast carcinoma cell line ZR-75-1. Compound K was added simultaneously with Erlotinib in 1:2.7 ratio combination a 4 day assay. Results are shown hereafter. Synergy was observed with CI<=0.59.

Compound K was added simultaneously with Erlotinib in 1:2.7 ratio in a 4 day assay. The experiment was performed in triplicate. The dose-response curves for single agents and combination are presented; see FIG. 54.

Compound K: IC50=4.1 uM; Maximum Concentration=75 uM

Erlotinib: IC50>200 uM; Maximum Concentration=200 uM

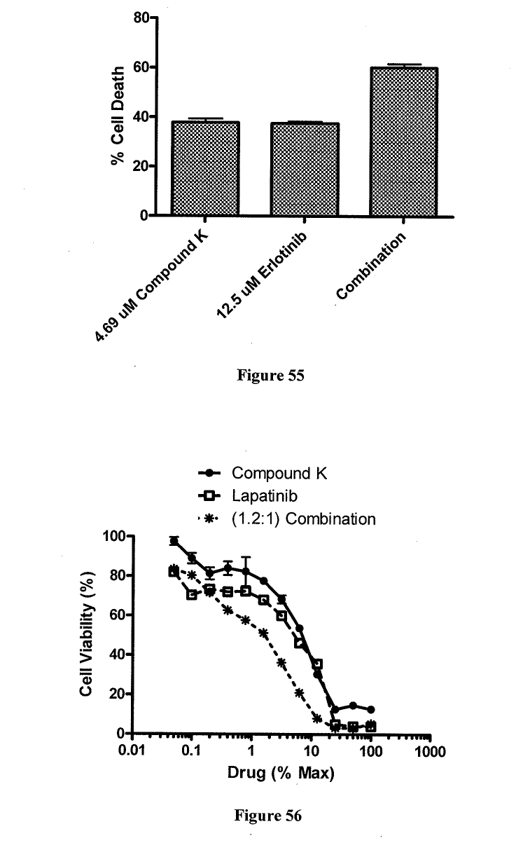

Combination: 50% Cell Death at 2.3 uM Compound K plus 6.2 uM Erlotinib; see FIG. 55.

CI=[IC50_{Combination}]/IC50_{Compound K}+[IC⁵⁰_{Combination}]/IC50_{Erlotinib}=(2.3/4.1)+(6.21>200)<=0.59.

### Example 26

### Lapatinib/Compound K Combination Testing in T47D Breast Carcinoma Cells

Lapatinib, a small molecule EGFR/Her2 inhibitor, was tested in combination with Compound K in the breast carcinoma cell line T47D. Compound K was added simultaneously with Lapatinib in 1:1.2 ratio combination a 4 day assay. Results are shown hereafter. Synergy was observed with CI=0.49.

Compound K was added simultaneously with Lapatinib in 1.2:1 ratio in a 4 day assay. The experiment was performed in triplicate. The dose-response curves for single agents and combination are presented; see FIG. 56.

Compound K: 1050=5.87 uM; Maximum Concentration=75 uM

Lapatinib: IC50=5.68 uM; Maximum Concentration=62.5 uM

Combination: 50% Cell Death at 1.53 uM Compound K plus 1.28 uM lapatinib

CI=[IC50_{Combination}]/IC50_{Compound K}+[IC50_{Combination}]/IC⁵⁰_{Lapalinib}=(1.53/5.87)+(1.28/5.68)=0.49.

### Example 27

### Sorafenib/Compound K Combination Testing in T47D Breast Carcinoma Cells

Sorafenib, a small molecule Raf/PDGFR/VEGFR2/VEGFR3/cKit inhibitor, was tested in combination with Compound K in the breast carcinoma cell line T47D. Compound K was added simultaneously with Sorafenib in 2:1 ratio combination a 4 day assay. Results are shown hereafter. Synergy was observed with CI=0.80.

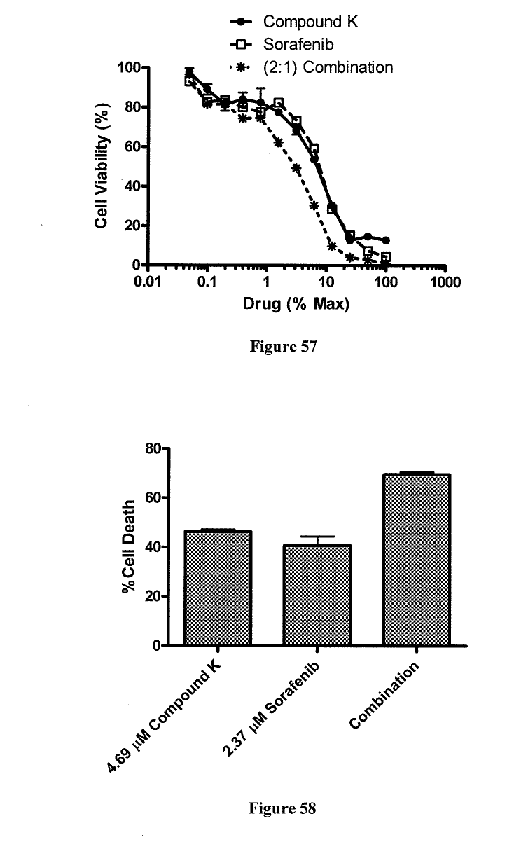

Compound K was added simultaneously with Sorafenib in 2:1 ratio in a 4 day assay. The experiment was performed in triplicate. The dose-response curves for single agents and combination are presented; see FIG. 57.

Compound K: IC50=5.87 uM; Maximum Concentration=75 uM

Sorafenib: IC50=3.58 uM; Maximum Concentration=37.5 uM

Combination: 50% Cell Death at 2.6 uM Compound K plus 1.3 uM Sorefenib; see FIG. 58.

CI=[IC50_{Combination}]/IC50_{Compound K}+[IC50_{Combination}]/IC50_{Sorafenibb}=(2.6/5.87)+(1.3/3.58)=0.80.

### Example 28

### Sunitinib/Compound K Combination Testing in T47D Breast Carcinoma Cells

Sunitinib, a small molecule inhibitor of multiple receptor tyrosine kinases, was tested in combination with Compound K in the breast carcinoma cell line T47D. Compound K was added simultaneously with Sunitinib in 1:1 ratio combination a 4 day assay. Results are shown hereafter. Synergy was observed with CI=0.86.

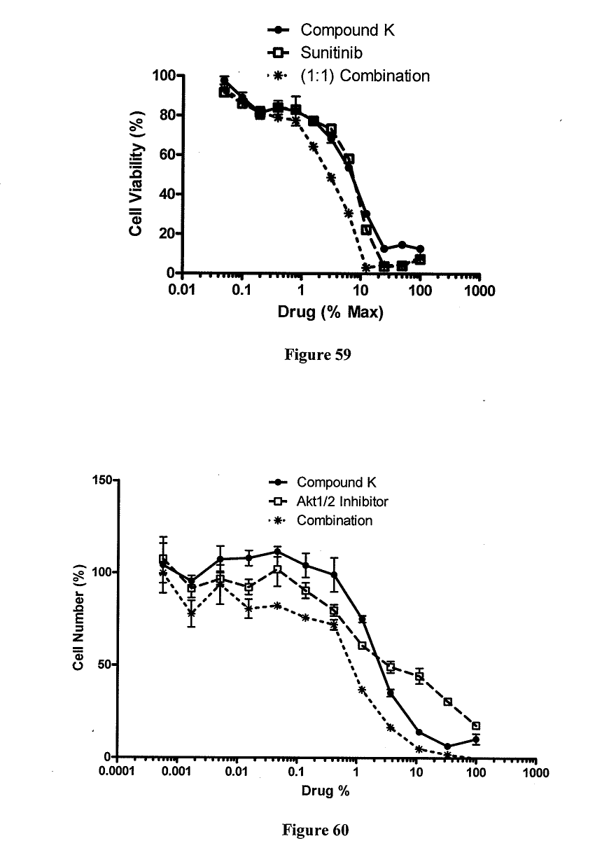

Compound K was added simultaneously with Sunitinib in 1:1 ratio in a 4 day assay. The experiment was performed in triplicate. The dose-response curves for single agents and combination are presented; see FIG. 59.

Compound K: IC50=5.87 uM; Maximum Concentration=75 uM

Sunitinib: IC50=6.2 uM; Maximum Concentration=75 uM

Combination: 50% Cell Death at 2.6 uM Compound K plus 2.6 uM Sunitinib

CI=[IC50_{Combination}]/IC50_{Compound K}+[IC50_{Combination}]/IC50_{Sunitinib}(2.6/5.87)+(2.65/6.2)=0.86.

### Example 29

### Akt 1/2 Inhibitor/Compound K Combination Testing in BT-474 Breast Carcinoma Cells

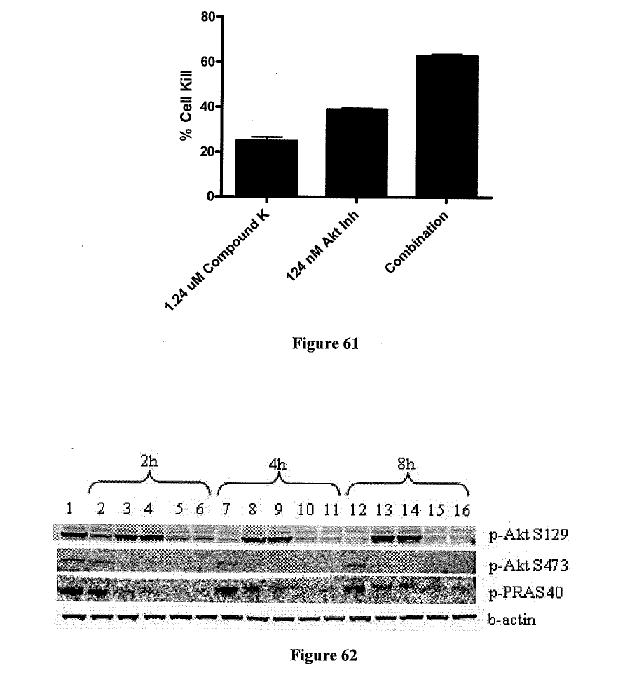

Isoform specific inhibitor of Akt1/2,1,3-Dihydro-1-(1-((4-(6-phenyl-1H-imidazo[4,5-g]quinoxalin-7-yl)phenyl)methyl)-4-piperidinyl)-2H-benzimidazol-2-one, was tested in combination with Compound K in the breast carcinoma cell line BT-474. Akt1/2 inhibitor was added simultaneously with Compound K in a 4 day assay. Results are shown hereafter; see FIG. 60 and FIG. 61. Synergy was observed with CI=0.38.

Akt1/2 inhibitor was added simultaneously with Compound K in 1:10 ratio in a 4 day assay. The experiment was performed in triplicate. The dose-response curves for single agents and combination are presented in FIG. 60.

Compound K: IC50=2.9 uM

Akt1/2 Inhibitor: IC50=0.5 uM

50% effect was achieved by combining 700 nM Compound K and 70 nM Akt1/2 Inhibitor.

CI=[IC50_{Combination}]/IC50_{Compound K}+[IC50_{Combination}]/IC50_{Akt1/2 Inhibitor}=(0.7/2.9)+(0.07/0.5)=0.38.

### Example 30

### Combination of Compound K with Erlotinib and Lapatinib in MDA-MB-453 Breast Carcinoma Cells

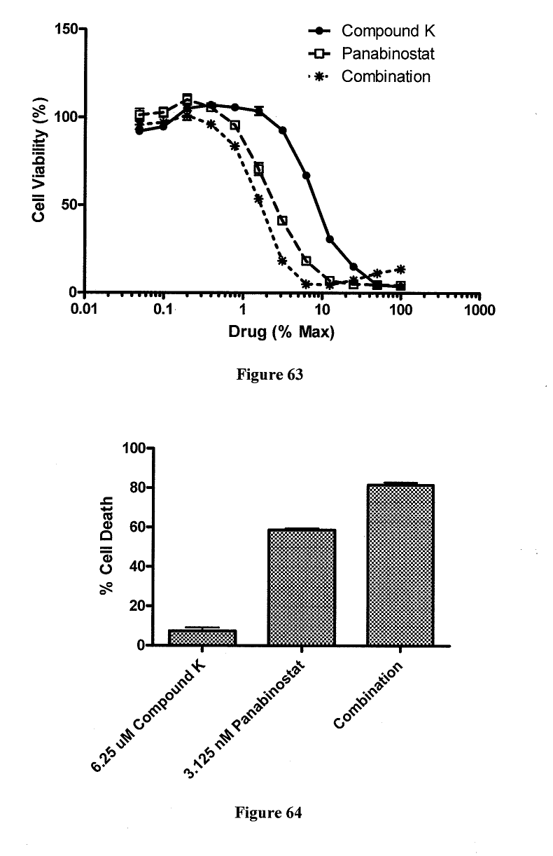

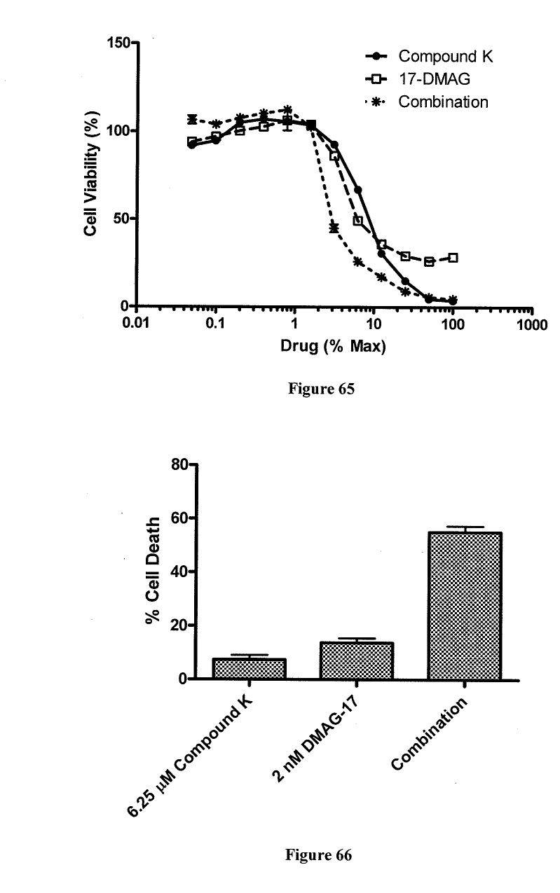

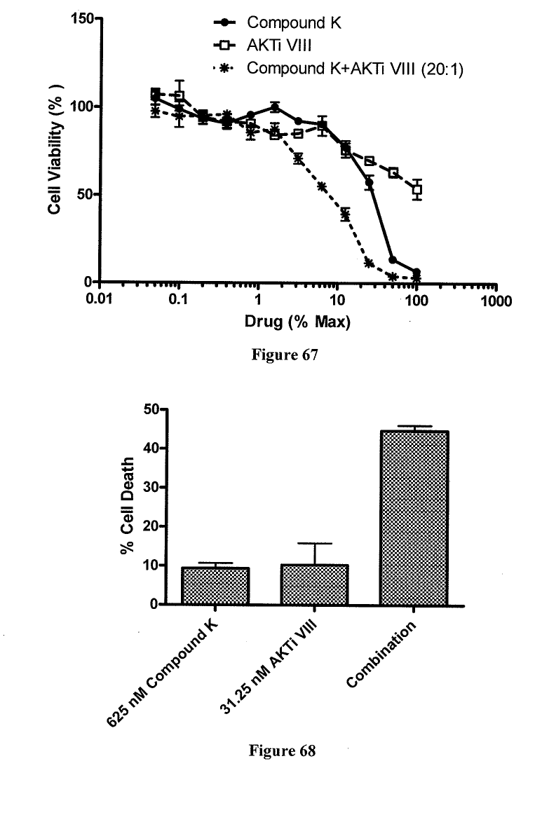

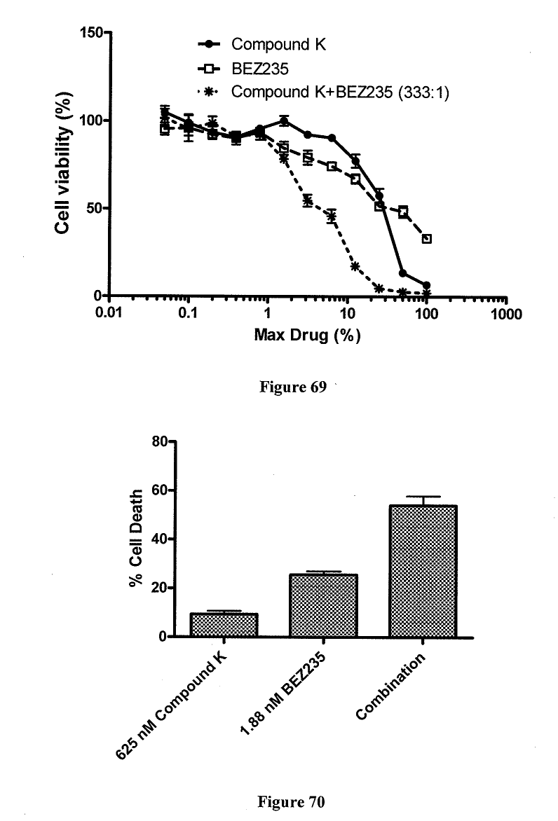

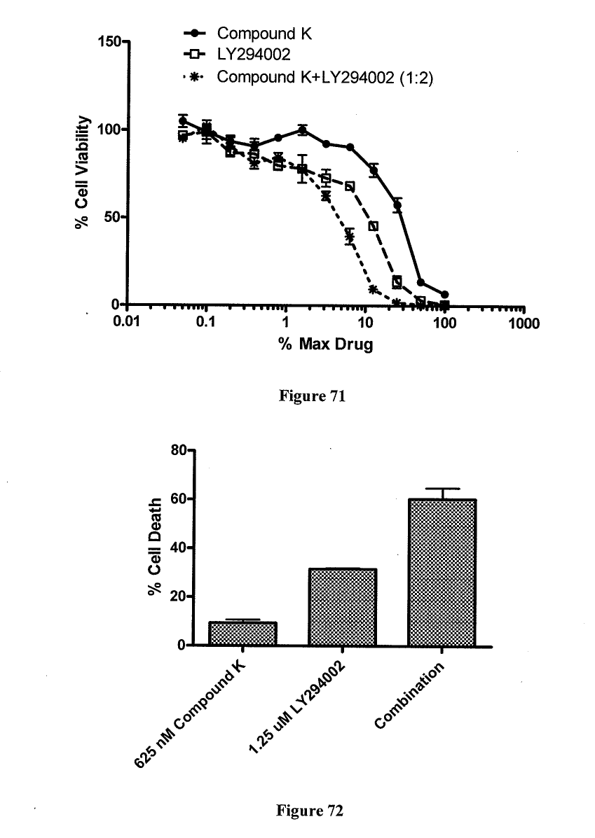

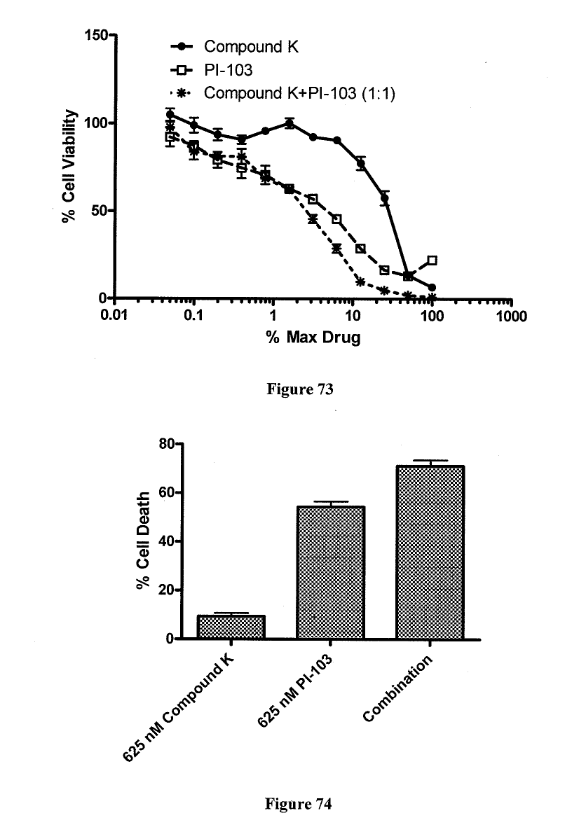

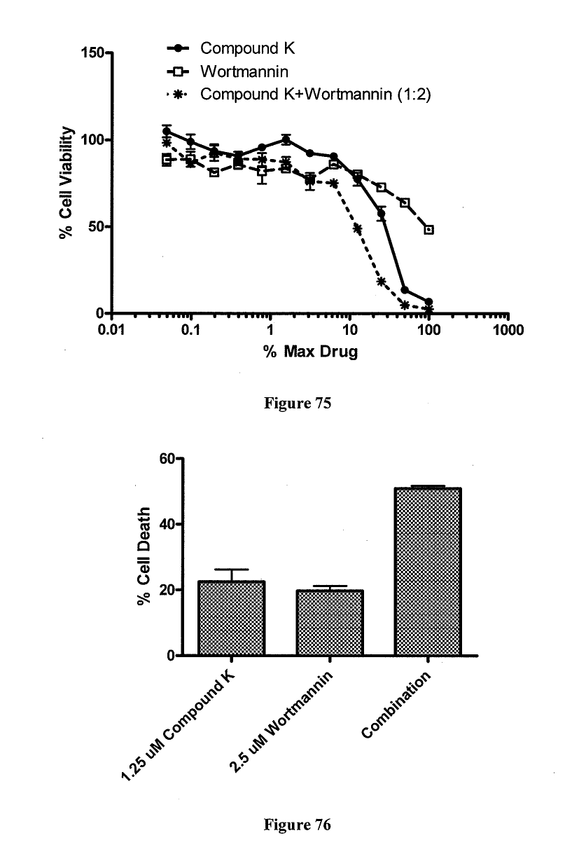

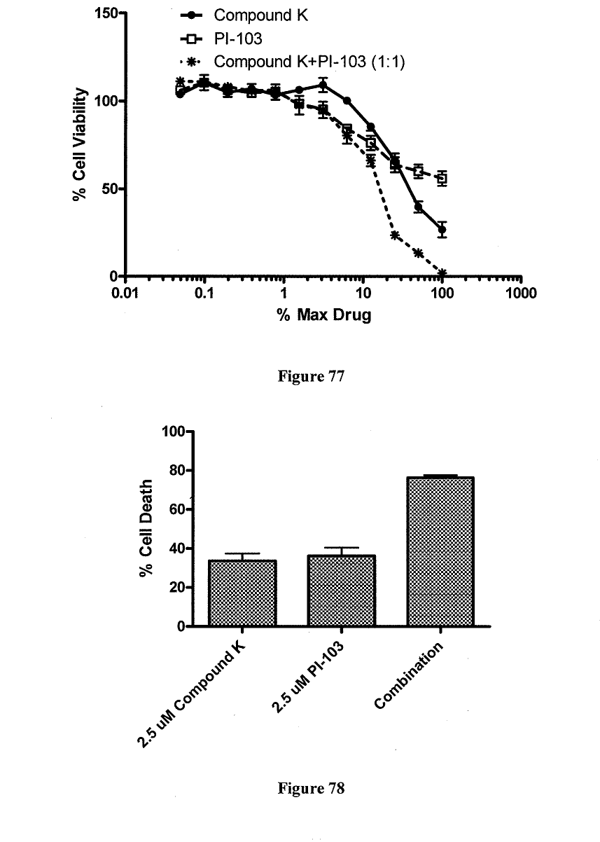

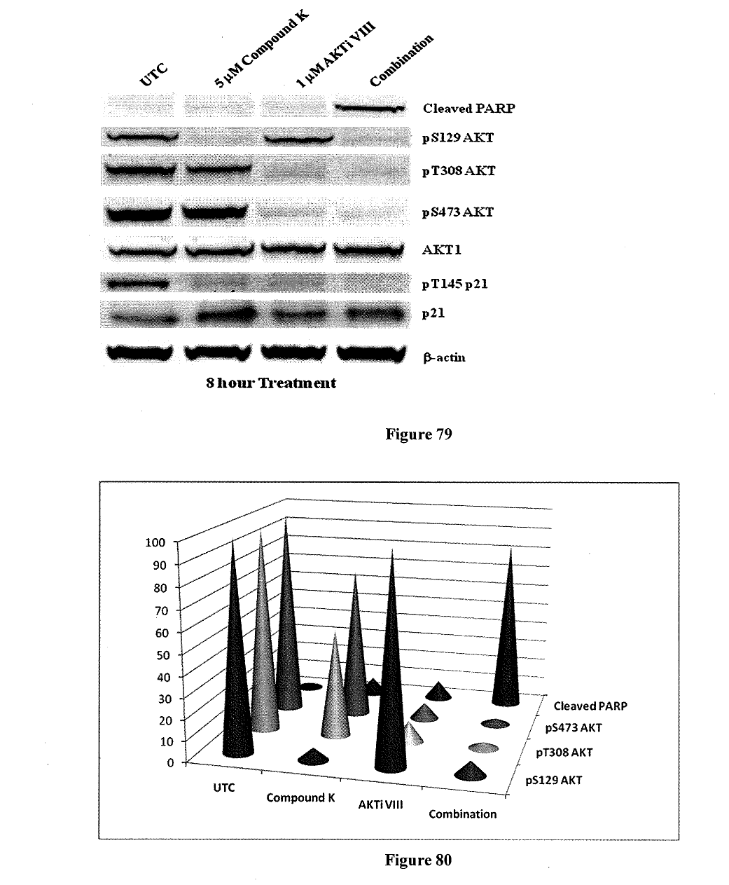

Small molecule inhibitors of EGFR, Erlotinib, and EGFR/Her2, Lapatinib, were tested as single agents or in combination with Compound K in the breast carcinoma cell line MDA-MB-453. 100 uM of Erlotinib or 2 uM Lapatinib were added simultaneously with 10 uM Compound K in a 2, 4 and 8 hour assays. Whole proteomes were isolated from treated cells and analyzed by Western blot for changes in phosphorylation status of Akt at Ser129 and Ser473 or downstream mediator of Akt activity, PRAS40 at Thr246. Results are shown in FIG. 62.

Treatment with Erlotinib or Lapatinib as single agents decreased phosphorylation of Akt at Ser473 below the detectable levels while having no effect on phosphorylation of Akt at Ser129. There was also a pronounced decrease in phosphorylation of PRAS40 at Thr246 at 2 and 4 hours, that was partially reversed by 8 hours.

Treatment with compound K as a single agent resulted in significant reduction of phosphorylation of Akt at Ser129 in a time-dependent manner. Phosphorylation of Akt at Ser473 was also affected, but to a lesser degree. The effect on phosphorylation of PRAS40 at Thr246 became evident at 4 and 8 hours, but was significantly less pronounced than for Erlotinib or Lapatinib.

Treatments with Compound K in combination with either Erlotinib or Lapatinib had similar effects on phosphorylation of Akt at Ser129 and Ser473 to single agents, but had more pronounced and sustained effect on phosphorylation of PRAS40 at Thr246 than any of the drugs alone.

Combination of Compound K with either Erlotinib or Lapatinib results in enhanced inhibition of Akt signaling.

### Example 31

### Panobinostat/Compound K Combination Testing in Hs 578T Breast Cancer Cells

Panobinostat, an HDAC inhibitor, was tested in combination with Compound K in the breast cancer cell line Hs 578T. Results are shown hereafter; see FIG. 63 and FIG. 64. Synergy was observed with CI50=0.76.

Panobinostat was added simultaneously with Compound K in 4 day assay. Drug/Drug molar ratio was 2000:1 (Compound K:Panobinostat). The experiment was performed in triplicate.

The dose-response curves for Compound K, Panobinostat and (2000:1) combination are presented in FIG. 63.

Compound K: 1050=17.63 uM; Maximum Concentration=200 uM

Panobinostat: IC50=2.76 nM; Maximum Concentration=100 nM

Combination: 50% Cell Death at 3.19 uM Compound K plus 1.6 nM Panobinostat.

CI50=[IC50_{Combination}]/IC50_{Compound K}+/[IC50_{Combination}]/IC50_{Panobinostat}=(3.19/17.63)+(1.6/2.76)=0.76.

### Example 32

### 17-DMAG/Compound K Combination Testing in Hs 578T Breast Cancer Cells

17-DMAG, an Hsp90 inhibitor, was tested in combination with Compound K in the breast cancer cell line Hs 578T. Results are shown hereafter; see FIG. 65 and FIG. 66. Synergy was observed with CI50=0.77.

17-DMAG was added simultaneously with Compound K in 4 day assay. Drug/Drug molar ratio was 3000:1 (Compound K:17-DMAG). The experiment was performed in triplicate.

The dose-response curves for Compound K, 17-DMAG and (3000:1) combination are presented in FIG. 65.

Compound K: IC50=16.71 uM; Maximum Concentration=200 uM

17-DMAG: 1050=6.37 nM; Maximum Concentration=66 nM

Combination: 50% Cell Death at 6.84 uM Compound K plus 2.28 nM 17-DMAG;.

CI50=[IC50_{Combination}]/IC50_{Compound K}+[IC50_{Combination}]/IC50_{17-DMAG}=(6.84/16.71)+(2.28/6.37)=0.77.

### Example 33

### AKTi VIII/Compound K Combination Testing in BT-474 Breast Cancer Cells

AKT inhibitor VIII (AKTi VIII, Akt1/2,1,3-Dihydro-1-(1-((4-(6-phenyl-1H-imidazo-[4,5-g]quinoxalin-7-yl)phenyl)methyl)-4-piperidinyl)-2H-benzimidazol-2-one (IC₅₀=58 nM, 210 nM, and 2.12 μM for Akt1, Akt2, and Akt3, respectively) was tested in combination with Compound K in the breast ductal carcinoma cell line BT-474. Results are shown hereafter; see FIGS. 67 and 68. Synergy was observed with CI50=0.37

AKTi VIII was added simultaneously with Compound K in 3 day assay. Drug/Drug molar ratios were 20:1 (Compound K/AKTi VIII). The experiment was performed in triplicate.

The dose-response curves for Compound K, AKTi VIII and (20:1) combination are presented in FIG. 67.

Compound K: IC50=2.68 uM; Maximum Concentration=10 uM

AKTi VIII: IC50=550 nM; Maximum Concentration=500 nM

Combination: 50% Cell Death at 786 nM Compound K plus 39.3 nM AKTi VIII

CI50=[IC50Combination]/IC50Compound K+[IC50Combination]/IC50AKYi VIII=(0.786/2.68)+(39.3/550)=0.37.

### Example 34

### Compound K/BEZ235 Combination Testing in BT-474 Breast Cancer Cells

BEZ235 (NVP-BEZ235), a PI3K/mTOR inhibitor, was tested in combination with Compound K in the breast ductal carcinoma cell line BT-474. Results are shown hereafter; see FIGS. 69 and 70. Synergy was observed with CI50=0.27

BEZ235 was added simultaneously with Compound K in 3 day assay. Drug/Drug molar ratios were 333:1 (Compound K/BEZ235). The experiment was performed in triplicate.

The dose-response curves for Compound K, BEZ235 and (333:1) combination are presented in FIG. 69.

Compound K: IC50=2.68 uM; Maximum Concentration=10 uM

BEZ235: IC50=10 nM; Maximum Concentration=30 nM

Combination: 50% Cell Death at 438 nM Compound K plus 1.3 nM BEZ235

CI50=[IC50Combination]/IC50Compound K+[IC50Combination]/IC50BEZ235=(0.438/2.68)+(1.3/10)=0.27.

### Example 35

### Compound K/LY294002 Combination Testing in BT-474 Breast Cancer Cells

LY294002, a PI3K inhibitor, was tested in combination with Compound K in the breast ductal carcinoma cell line BT-474. Results are shown hereafter; see FIGS. 71 and 72. Synergy was observed with CI50=0.61

LY294002 was added simultaneously with Compound K in 3 day assay. Drug/Drug molar ratios were 1:2 (Compound K/LY294002). The experiment was performed in triplicate.

The dose-response curves for Compound K, LY294002 and (1:2) combination are presented in FIG. 71.

Compound K: IC50=2.68 uM; Maximum Concentration=10 uM

LY294002: IC50=2.26 uM; Maximum Concentration=20 uM

Combination: 50% Cell Death at 482 nM Compound K plus 964 nM LY294002

CI50=[IC50Combination]/IC50Compound K+[IC50Combination]/IC50LY294002=(0.482/2.68)+(0.964/2.26)=0.61.

### Example 36

### Compound K/PI-103 Combination Testing in BT-474 Breast Cancer Cells

PI-103, a PI3K/mTOR inhibitor, was tested in combination with Compound K in the breast ductal carcinoma cell line BT-474. Results are shown hereafter; see FIGS. 73 and 74. Synergy was observed with CI50=0.82

PI-103 was added simultaneously with Compound K in 3 day assay. Drug/Drug molar ratios were 1:1 (Compound K/PI-103). The experiment was performed in triplicate.

The dose-response curves for Compound K, PI-103 and (1:1) combination are presented in FIG. 73.

Compound K: IC50=2.68 uM; Maximum Concentration=10 uM

PI-103: IC50=410 nM; Maximum Concentration=10 uM

Combination: 50% Cell Death at 293 nM Compound K plus 293 nM PI-103

CI50=[IC50Combination]/IC50Compound K+[IC50Combination]/IC50PI-103=(0.293/2.68)+(293/410)=0.82.

### Example 37

### Compound K/Wortmannin Combination Testing in BT-474 Breast Cancer Cells

Wortmannin, a PI3K inhibitor, was tested in combination with Compound K in the breast ductal carcinoma cell line BT-474. Results are shown hereafter; see FIGS. 75 and 76. Synergy was observed with CI50=0.59

Wortmannin was added simultaneously with Compound K in 3 day assay. Drug/Drug molar ratios were 1:2 (Compound K/Wortmannin). The experiment was performed in triplicate.

The dose-response curves for Compound K, Wortmannin and (1:2) combination are presented in FIG. 75.

Compound K: IC50=2.68 uM; Maximum Concentration=10 uM

Wortmannin: IC50=25.92 uM; Maximum Concentration=20 uM

Combination: 50% Cell Death at 1.3 uM Compound K plus 1.3 uM Wortmannin

CI50=[IC50Combination]/IC50Compound K-1-[IC50Combination]/IC50Wortmannin=(1.3/2.68)+(1.3/25.92)=0.59.

### Example 38

### Compound K/PI-103 Combination Testing in T-47D Breast Cancer Cells

PI-103, a PI3K/mTOR inhibitor, was tested in combination with Compound K in the breast ductal carcinoma cell line T-47D. Results are shown hereafter; see FIGS. 77 and 78. Synergy was observed with CI50=0.66

PI-103 was added simultaneously with Compound K in 3 day assay. Drug/Drug molar ratios were 1:1 (Compound K/PI-103). The experiment was performed in triplicate.

The dose-response curves for Compound K, PI-103 and (1:1) combination are presented in FIG. 77.

Compound K: IC50=3.35 uM; Maximum Concentration=10 uM

PI-103: IC50=5.37 uM; Maximum Concentration=10 uM

Combination: 50% Cell Death at 1.37 uM Compound K plus 1.37 uM PI-103

CI50=[IC50Combination]/IC50Compound K+[IC50Combination]/IC50PI-103=(1.37/3.35)+(1.37/5.37)=0.66.

### Example 39

### Compound K/AKTi VIII Combination Testing in BT-474 Breast Cancer Cells

AKT inhibitor VIII (AKTi VIII, 3-Dihydro-1-((4(4-(6-phenyl-1H-imidazo-[4,5-g]quinoxalin-7-yl)phenyl)methyl)-4-piperidinyl)-2H-benzimidazol-2-one (IC50=58 nM, 210 nM, and 2.12 μM for Akt1, Akt2, and Akt3, respectively) was tested in combination with Compound K in the breast ductal carcinoma cell line BT-474. Results are shown hereafter; see FIGS. 79 and 80. Synergy induction of apoptosis was observed.

AKTi VIII was added simultaneously with Compound K in 8 hour assay. Drug/Drug molar ratios were 5:1 (Compound K/AKTi VIII).

The western hybridization analysis for untreated cells (UTC), Compound K, AKTi VIII and (5:1) combination are presented in FIG. 80.

Compound K: Dramatically reduced phosphorylation of AKT at S129, had moderate effect on phosphorylation of AKT at T308 and S473. Dramatically decreased phosphorylation of p21 at T145. Had very minor effect on cleavage of PARP (i.e. induction of apoptosis).

AKTi VIII: Had no effect on phosphorylation of AKT at S129, Dramatically reduced phosphorylation of AKT at T308 and S473. Dramatically decreased phosphorylation of p21 at T145. Had very minor effect on cleavage of PARP (i.e. induction of apoptosis).

Combination: Dramatically reduced phosphorylation of AKT at S129, T308 and S473. Further decreased phosphorylation of p21 at T145. Had major effect on cleavage of PARP (i.e. induction of apoptosis).

Combination of Compound K with AKTi VIII inhibits phosphorylation of AKT at S129, T308, S473 and synergistically induces apoptosis (as demonstrated by cleavage of PARP).

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A method for treating or ameliorating a neoplastic disorder, comprising administering to a subject in need thereof a therapeutically effective amount of a compound of formula I: 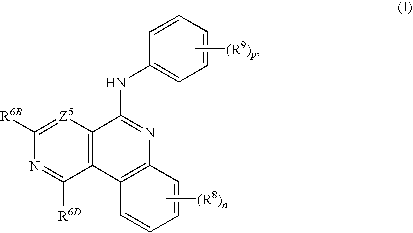
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4;
and an anticancer agent, thereby treating or ameliorating said neoplastic disorder.

2. The method of claim 1, wherein the compound of formula I has the structure: 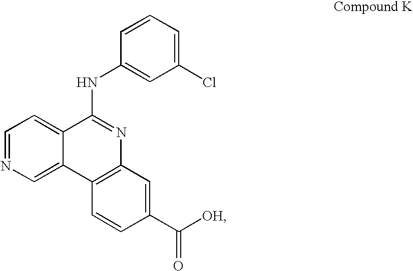
or a pharmaceutically acceptable salt or ester thereof.

3. The method of claim 1, wherein the anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor.

4. The method of claim 1, wherein the anticancer agent is selected from the group consisting of 5-fluorouracil (5-FU), cisplatin, doxorubicin, fludarabine, gemcitabine, paclitaxel, rapamycin, sunitinib, lapatinib, sorafenib, erlotinib, vinblastine, 1,3-Dihydro-1-(1-((4-(6-phenyl-1H-imidazo [4,5-g]quinoxalin-7-yl)phenyl)methyl)-4-piperidinyl)-2H-benzimidazol-2-one, panobinostat, 17-DMAG, BEZ-235, LY294002, PI-103, and wortmannin.

5. The method of claim 1, wherein the neoplastic disorder is cancer.

6. The method of claim 5, wherein the cancer is cancer of the hemopoietic system, lung, breast, prostate, kidney, pancreas, liver, heart, skeleton, colon, rectum, skin, brain, eye, lymph node, heart, testes or ovary.

7. The method of claim 1, wherein the compound of formula I and the anticancer agent are administered simultaneously.

8. The method of claim 1, wherein the compound of formula I and the anticancer agent are administered simultaneously and separately.

9. The method of claim 1, wherein the compound of formula I and the anticancer agent are administered sequentially.

10. The method of claim 9, wherein the compound of formula I is administered prior to the anticancer agent.

11. The method of claim 9, wherein the compound of formula I is administered after the anticancer agent.

12. The method of claim 1, wherein the subject is human.

13. The method of claim 1, wherein the compound of formula I and the anticancer agent provide at least an additive anticancer effect.

14. The method of claim 1, wherein the compound of formula I and the anticancer agent provide a synergistic anticancer effect.

15. A method for inhibiting cell proliferation in a system, comprising administering to the system an effective amount of a compound of Formula I: 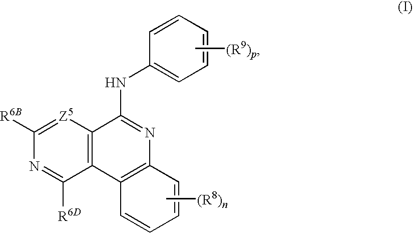
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4;
and an anticancer agent or a pharmaceutically acceptable salt or ester thereof; thereby inhibiting cell proliferation.

16. The method of claim 15, wherein the system is a cell, tissue or subject.

17. The method of claim 15, wherein the compound of formula I has the structure: 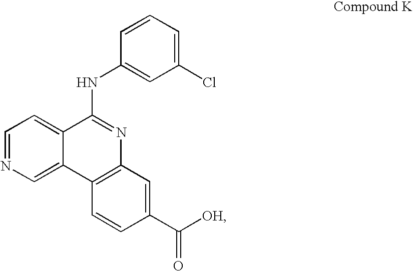
or a pharmaceutically acceptable salt or ester thereof.

18. The method of claim 15, wherein the anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor.

19. A pharmaceutical composition comprising a compound of formula I: 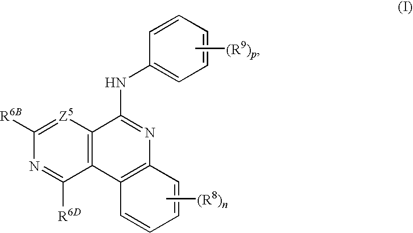
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4;
an anticancer agent, and at least one pharmaceutically acceptable excipient.

20. The composition of claim 19, wherein the compound of Formula I has the structure: 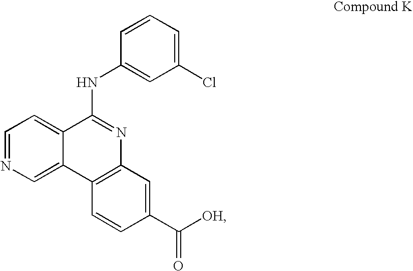
or a pharmaceutically acceptable salt or ester thereof.

21. The composition of claim 19, wherein the anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor.

22. The composition of claim 20, wherein the anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor.

23. The method of claim 1, wherein the compound of Formula I has the structure of Formula II, III, IV, V or VI: 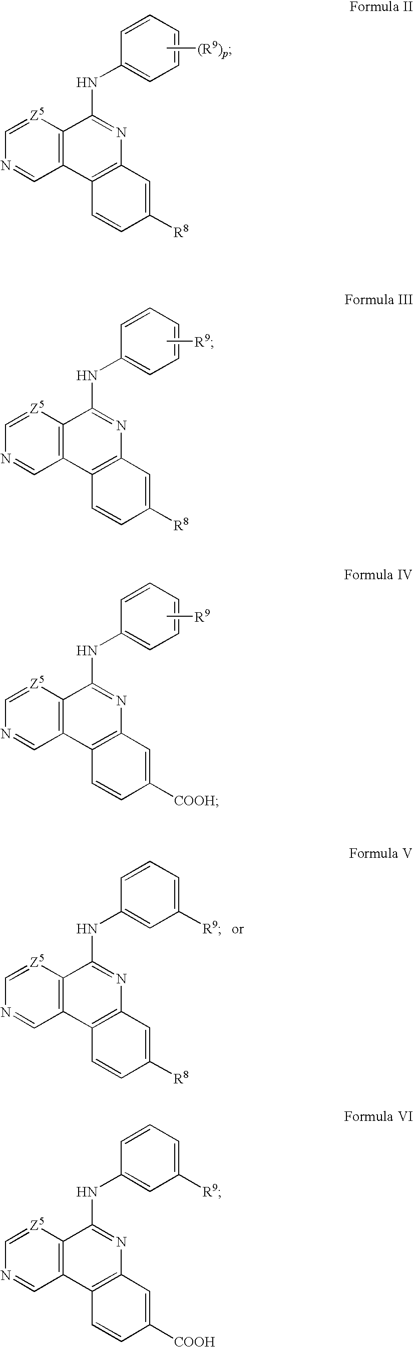
or a pharmaceutically acceptable salt or ester thereof;
wherein Z⁵ is N or CR^{6A};
each R^{6A} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and =0;
and wherein two R′ can be linked to form a 3-7 membered ring optionally containing up to three heteroatoms selected from N, O and S; and
p is 0 to 4.

24. The method of claim 15, wherein the compound of Formula I has the structure of Formula II, III, IV, V or VI: 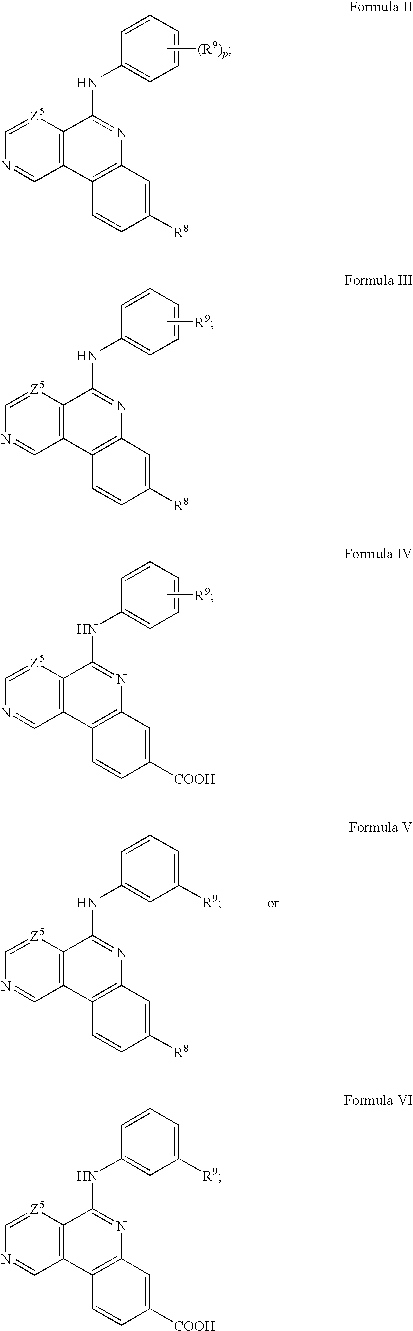
or a pharmaceutically acceptable salt or ester thereof;
wherein Z⁵ is N or CR^{6A};
each R^{6A} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂, wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S; and
p is 0 to 4.
